(19) United States
(12) Patent Application Publication
(10) Pub. No.: US 2021/0276960 A1
(43) Pub. Date: Sep. 9, 2021

(54) INHIBITORS OF TISSUE TRANSGLUTAMINASE 2 (TG2) AND USES THEREOF

(71) Applicants: Northwestern University, Evanston, IL (US); United States Goverment as represented by the Department of Veterans Affairs, Washington, DC (US)

(72) Inventors: Gary E. Schiltz, Naperville, IL (US); Daniela E. Matei, Chicago, IL (US); Purav Vagadia, Aurora, IL (US)

(21) Appl. No.: 17/249,519

(22) Filed: Mar. 3, 2021

Related U.S. Application Data
(60) Provisional application No. 62/985,269, filed on Mar. 4, 2020.

(51) Int. Cl. C07D 239/48; A61P 35/00; C07D 239/42; C07D 401/12; C07D 403/12; C07D 409/12

(52) U.S. Cl. CPC C07D 239/48; A61P 35/00; C07D 409/12; C07D 401/12; C07D 403/12; C07D 239/42

(57) ABSTRACT

Disclosed are compounds, pharmaceutical compositions comprising the compounds, and methods of using the compounds and pharmaceutical compositions for treating a subject in need thereof. The disclosed compounds inhibit the activity of tissue transglutaminase 2 (TG2). As such, the disclosed compounds and pharmaceutical compositions may be utilized in methods for treating a subject having or at risk for developing a disease or disorder that is associated with TG2 activity which may be cell proliferative diseases and disorders such as cancer.

### CROSS-REFERENCED TO RELATED PATENT APPLICATIONS

This application claims the benefit of priority under 35 U.S.C. § 119(e) to U.S. Provisional Application No. 62/985,269, filed Mar. 4, 2020, the contents of which are incorporated herein by reference in their entireties.

### STATEMENT REGARDING FEDERALLY SPONSORED RESEARCH OR DEVELOPMENT

This invention was made with government support under grant 101 BX000792-06 awarded by US Department of Veterans Affairs and grant CA207288 and CA060533 awarded by the National Institutes of Health. The government has certain rights in the invention.

### BACKGROUND

The field of the invention relates to small molecule inhibitors of the interaction between tissue transglutaminase 2 (TG2) and fibronectin. In particular, the field of the invention relates to small molecule inhibitors of the interaction between TG2 and fibronectin which may be formulated as pharmaceutical compositions for treatment of cell proliferative diseases and disorders such as cancer.

In some aspects, the disclosed subject matter relates to methods for treating cancers such as ovarian cancer. Ovarian cancer (OC) is the leading cause of death among gynecological malignancies¹ and metastasis to abdominal organs is the most common cause of OC mortality. Importantly, OC metastasizes by direct exfoliation of cancer cells from the primary tumor and intraperitoneal (ip) dissemination, without the requirement for vascular invasion. Adhesion to the extracellular matrix (ECM) plays an important role in the initiation of peritoneal metastasis. We identified tissue transglutaminase (TG2), an enzyme involved in Ca⁺⁺-dependent protein post-translational modifications and which has a well-defined binding site for fibronectin (FN), as being highly expressed in OC².

The present inventors have shown that TG2 critically regulates the process of OC metastasis²⁻⁴. The inventors have shown showed when injected ip or under the ovarian bursa of nude mice, OC cells engineered to express decreased levels of TG2 disseminated less efficiently compared to control cells. These results of these studies have the interaction between TG2 and fibronectin (FN) as being a critical component in this process. The inventors have demonstrated that the TG2/FN complex is implicated in OC metastasis via multiple mechanisms: a) adhesion to the ECM by strengthening of integrin-dependent cell-matrix adhesion², b) induction of epithelial to mesenchymal transition (EMT)^{5,6}, c) regulation of Wnt/β-catenin signaling 7, through direct interaction with the Frizzled 7 (Fzd7) receptor, which in turn promotes cancer cell proliferation and persistence of a stem cell profile⁸, d) remodeling of the extracellular matrix⁴, and e) modulation of intracellular oncogenic signaling^{7,9}.

Based on these results, the inventors hypothesized that the TG2/FN interaction is targetable and that its disruption by small molecules will prevent cancer cell adhesion to the matrix and OC metastasis. The inventors used preliminary computational¹⁰ and high throughput screening (HTS) strategies to discover several promising compounds¹¹ that potently blocked this protein-protein interaction (PPI). The inventors propose that the characterization of these compounds and biochemical optimization could lead to a new therapeutic strategy in OC. Furthermore, the inventors hypothesize that the use of small molecule inhibitors (SMI) will reveal novel mechanisms by which TG2/FN interaction affects OC metastasis and stemness.

To this end, we completed HTS by using a newly developed Alpha-Lisa assay that robustly measured the TG2/FN complex formation and identified a new class of diamino-pyrimidines as potent inhibitors for this PPI¹¹. The inventos have demonstrated that several of these small molecules potently blocked OC cell adhesion and migration. By using structure-activity relationship (SAR) strategies, the inventors optimized the structure of the lead compound and generated analogs with improved efficacy.

### SUMMARY

Disclosed are compounds, pharmaceutical compositions comprising the compounds, and methods of using the compounds and pharmaceutical compositions for treating a subject in need thereof. The disclosed compounds inhibit the activity of tissue transglutaminase 2 (TG2). As such, the disclosed compounds and pharmaceutical compositions may be utilized in methods for treating a subject having or at risk for developing a disease or disorder that is associated with TG2 activity which may be cell proliferative diseases and disorders such as cancer.

The disclosed compounds, compositions and related methods of use for the selective inhibition of tissue transglutaminase 2 (TG2) may be performed in vivo, for example in methods of treating diseases or disorders associated with TG2 activity. The disclosed compounds, compositions and related methods of use for the selective inhibition of tissue transglutaminase 2 (TG2) also may be performed in vitro.

In some embodiments, the disclosed compounds have a formula I:

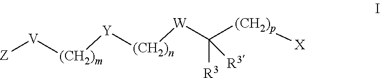

or a salt or solvate thereof,
wherein:
Z is pyrimidinyl (e.g., pyrimidin-2-yl) or 5,7-diazaindolinyl (e.g., 5,7-diazaindolin-6-yl) and Z optionally is substituted at one or more ring positions with alkyl (e.g., methyl), alkoxy (e.g., methoxy), amino, alkylamino (e.g., methylamino), dialkylamino (e.g., dimethylamino), heteroaryl (e.g., a nitrogen-containing heteroaryl group such as pyrrolidinyl and in particular N-pyrrolidinyl), or halo (e.g., chloro);
V is O or NR¹, wherein R¹ is hydrogen or alkyl (e.g., methyl);
m is selected from 0-6;
Y is an aryl group (e.g., phenyl), a diaryl group (e.g., diphenyl), or a heteroaryl group (e.g, pyridinyl such as pyridin-2-yl or pyridine-3-yl, or thiophenyl such as thiophen-2-yl or thiophen-5-yl), and Y optionally is substituted at one or more ring positions with haloalkyl (e.g., trifluoromethyl);
n is selected from 0-6;
W is CH or NR², wherein R2 is hydrogen or alkyl (e.g., methyl);
R³ and R^{3′} are hydrogen or R³ and R^{3′} together form oxo;
p is selected from 0-6; and
X is an aryl group (e.g., phenyl or naphthyl such as naphth-1-yl) or a heteroaryl group (e.g, pyridinyl such as pyridin-2-yl or pyridin-3-yl or pyridine-4-yl, piperazinyl such as piperazin-1-yl, thiophenyl such as thiophen-2-yl, pyrazol such as pyrazol-1-yl, and indolyl such as indol-3-yl), and X optionally is substituted at one or more ring positions with one or more of alkyl (e.g., ethyl), alkoxy (e.g., methoxy), halo (e.g., chloro); amino; alkylamino (e.g., methylamino), or aryl (e.g., phenyl); and
optionally with the proviso that if m is 0, n is 0, and p is 1, then X is not N-imidazyl or indol-3-yl; and
optionally with the proviso that if m is 0, n is 0, and p is 1, then Z is not 4-dimethylamino-pyrimidin-2-yl or 4-pyrrolidin-1-yl-pyrimidin-2-yl.

The disclosed compounds may be formulated as pharmaceutical compositions comprising the compounds or pharmaceutically acceptable salts thereof in a pharmaceutically acceptable carrier for use in treatment methods for a subject in need thereof. In some embodiments, the disclosed compounds and pharmaceutical compositions may be utilized to treat diseases or disorders associated with tissue transglutaminase (TG2) activity and/or expression. Particularly, the disclosed compounds and pharmaceutical compositions may be utilized to inhibit the interaction of TG2 and fibronectin in a subject in need thereof and treat diseases or disorders that are associated with TG activity and or expression. In some embodiments, the disclosed compounds and pharmaceutical compositions may be utilized to treat cell proliferative diseases or disorders in a subject in need thereof, including cancers such as ovarian cancer.

### BRIEF DESCRIPTION OF THE FIGURES

FIG. 1. Chemical structures of TG53 (parent compound) and of the 6 derivatives targeting the TG2-FN protein-protein interaction (MT1-MT6).

FIG. 2. TG53 derivatives inhibit TG2-FN interaction. A, ELISA measured the interaction between His-tagged TG2 and biotinylated FN42 in the presence of vehicle (DMSO, control), TG53 and MT1-MT6 at 10 μM and 25 μM concentrations. Bars represent means±SD (n=3). B, Bio-layer Interferometry (BLI) sensograms monitor the real time association and dissociation kinetics of FN45 (captured on streptavidin-coated sensors) and TG2. The TG2 concentrations used at the association step are indicated. The nonlinear regression fits from 1:1 global analysis are shown as thin black lines; kₐ=5320 M⁻¹s⁻¹ (±0.2%), kₐ=0.0016 s⁻¹ (±0.4%), yielding K_{D}=0.30 nM; goodness of fit: R²=0.996084. C, Concentration-dependent inhibitory effect of MT4 on the FN45-TG2 interaction. For each MT4 concentration tested, the extent of binding (Rmax) was corrected for nonspecific binding of (TG2+MT4) to bare sensors. D, Bio-layer Interferometry (BLI) sensograms show the real time association and dissociation kinetics of FN45 (20 μg/ml captured on streptavidin-coated sensors) and 1 μM TG2 pre-incubated with MT4 at various concentrations. E, Solid phase assay measured SKOV3 cells' adhesion to FN (5 μg/mL) in the presence of TG53 and MT1-6 (n=4). F-H, Dose-dependent effect of MT4 (2 μM-25 μM) on SKOV3 (F), OVCAR433 (G), and OVCAR5 (H) cells adhesion onto FN-coated plates was measured by a solid phase assay (see SM). For all experiments, results represent the mean and SD of at least triplicate samples: *p<0.05, **p<0.01, ***p<0.001, ****p<0.0001.

FIG. 3. TG53 inhibits formation of a complex with integrin β1 and activation of FAK and c-Src during SKOV3 cell attachment onto FN. A-D, Confocal microscopy analysis of TG2 (Cy5, red; x600) co-localization with integrin β1, p-FAK (Y576/577), c-Src, and p-c-Src (Y416) (Alexa Fluor 488) in the presence of either 1 μM TG53 (lower panels) or DMSO vehicle (upper panels). Graphs quantifying co-localization of the two proteins are presented. *p<0.05, **p<0.01.

FIG. 4. MT-4 inhibits early OVCAR5 cell attachment onto FN by interfering with the formation of stable focal contacts. A-C, IF staining of adhesion complex factors in OVCAR5 cells upon cell adhesion to FN. A, Confocal imaging of intracellular localization of vinculin-positive focal adhesion points (white, upper panel), pFAK (white, lower panel), and actin filaments (upper and lower panel) at 30 minutes after seeding of OVCAR5 cells onto FN coated chamber slides in the presence or absence of inhibitors (TG53 and MT-4 at 1 μM concentration) or vehicle (DMSO). Cells were treated with the SMIs for 72 hours prior to seeding and during the assay. Insets provide detailed view of the vinculin-positive structures. Scale bar=10 μm. B, Analysis of the actin cytoskeleton assessed morphological differences during cell attachment. Scale bar=10 μm. C, Grayscale images of actin cytoskeleton in TG53- (left) and MT4-treated cells (right). Scale bar=20 μm. D, TIRF microscopy analysis of OVCAR5 cells pre-treated with SMIs for 1 h to determine the distribution of integrin β1 (red, upper panels) at the interface with FN during cell attachment. Actin cytoskeleton is depicted (lower panels). Scale bar=10 μm.

FIG. 5. Effects of SMIs targeting the TG2/FN complex on “outside-in” signaling and adhesion to mesothelial surfaces. A, Western blot analysis of total and phosphorylated FAK and ERK was performed using cell lysates extracted from OVCAR cells plated on FN for 15 to 45 minutes in the presence of TG53 (left) or MT-4 (right) at 5 μM concentration, as compared to DMSO. GAPDH was used as a control. OVCAR5 cells had been pretreated with the inhibitors for 72 hours. Densitometry analysis used the Quantity One software and results were normalized to GAPDH loading controls. DMSO 0 min time point was considered as 1 for comparative analysis. Values are shown for each band. B, A modified cell adhesion assay measured the effects of TG53, MT-4, MT-5, and MT-6 (25 μM) on SKOV3 cells' adhesion onto semi-confluent LP9 monolayers. C, Dose-dependent effects of TG53, MT-4, MT-5, and MT-6 on OVCAR433 cells adhesion onto semi-confluent LP9 monolayers. D-E, Wound healing assay measures migration of SKOV3 and OVCAR5 cells in the presence of MT-4, MT-5, MT-6 at a concentration of 8 μM (black bars) or vehicle (open bar). Migration rate was determined over a period of 24 hours by measuring the average distance between the wound borders at the beginning and at the end of the assay interval (n=4). F-G, SKOV3-GFP OC cells were injected ip in NSG mice in the presence of either MT-4 (l0 g/kg) (F) or MT-6 (l0 g/kg) (G) and allowed to attach to the peritoneal wall. After 2 hours, the cell suspension was recovered from the peritoneal cavity through peritoneal washings and the non-attached OC cells recovered were counted. *p<0.05, **p<0.01, ***p<0.001, ****p<0.0001.

FIG. 6. Priming of OVCAR5 cells with MT-4 sensitizes cells to paclitaxel (PTX). A-B, The CCK8 assay was used to measure cell viability of SKOV3 cells treated with increasing concentrations of TG53 and MT1-6 for 72 hours. C-D, Colony formation from OVCAR-5 cells pre-treated with 1 μM MT-4 or 1 μM TG53 for 72 hours before seeding in the presence or absence of 5 nM PTX (n=6; 10 days incubation to allow colonies formation). Colonies were stained with 0.4% crystal violet and counted using ImageJ. E-F, Sphere formation assay measured proliferation of OVCAR-5 cells pre-treated with 1 μM MT-4 or 1p M TG53. Cells were allowed to form spheroids in the presence or absence of 5 nM PTX (n=3). Spheres were counted after ten days. *p<0.05, **p<0.01, ***p<0.001, ****p<0.0001.

FIG. 7. Schematic illustration of TG2 domains; a N-terminal β-sandwich domain, an u/p catalytic core domain containing Cys²⁷⁷, His³³⁵, and Asp³⁵⁸ catalytic triad, and 2 C-terminal β-barrel domains.

FIG. 8. TG2 knock down decreases tumor formation and metastasis in an OC orthotopic model. A. Western blotting for TG2 in SKOV3 cells stably transfected with AS-TG2 and pcDNA3.1 control vector. B. Mean tumor volumes derived from pcDNA3.1 or ASTG2 cells (p=0.01). C Number of mice developing disseminated metastases (>50 implants) after control and ASTG2 cells' implantation under the ovarian bursa (p=0.02)

FIG. 9. A) PLA detects TG2-integrin β1 complex formation in ovarian cancer and normal oviduct. B-C) Correlation between TG2 and integrin β1 (ITGB1) mRNA expression levels (R=0.23; P<0.0001) and between TG2 and FN1 mRNA expression levels (R=0.39; P<0.0001) in the OC Agilent 244K TCGA database. D. Overall survival curves generated using the Kaplan-Meier plot for tumors expressing high levels of TG2 and of ITGB1 vs. those expressing low levels of TG2 and of ITGB1 vs. all others in the Agilent 244K TCGA database (P=0.00652). The median survival time for each group is presented in brackets.

FIG. 10. Ovarian CSCs express high levels of TG2, Integrin 01, and FN1. A-B. TG2, integrin β1 (ITGB1), and FN1 mRNA levels measured by quantitative real-time PCR in ALDH⁺/CD133⁺ vs. ALDH⁻/CD133⁻ isolated from human ovarian cancer OVCAR5 and COV362 cells. (N≥3; *P<0.05, **P<0.01). C-D. Expression of TG2, integrin β1 (ITGB1), and FN1 in OVCAR5 and COV362 cells grown as monolayers or spheroids under low adherence conditions. (N≥3; *P<0.05, **P<0.01). E. Western blotting shows TG2, integrin 1, and FN expression levels in OVCAR5, COV362, and SKOV3 grown as monolayers (m) and spheroids (s). Densitometry quantifies TG2, integrin β1, and FN expression levels normalized for GAPDH. F. Western blot shows TG2 expression levels in OVCAR5-control cells compared to TG2-KO clones (2D2 and 2H5). G. FACS measures ALDEFLUOR-FITC⁺/CD133-APC⁺ cells in OVCAR5 control and TG2 knock out clones. DEAB/APC-Isotype treated cells are negative controls.

FIG. 11. Characteristics of the AlphaLISA™ assay. A, Design of the AlphaLISA assay. Donor beads coated with streptavidin and acceptor nickel chelate beads were used to capture biotinylated FN42 and His tagged TG2 protein. After excitation at 680 nm singlet oxygen is transferred from donor to acceptor beads coming within a distance of 200 nm and results in light emitted by the acceptor beads. B, Cross-titration was performed to optimize detection of the TG2-FN interaction by the assay. Saturation isotherms of FN42 binding to TG2 were generated. The K_{d} was 2.43 nM. C, Titration curves represented with GraphPad Prism demonstrate reaching the hook point at 3 nM biotinylated FN42 and 10 nM TG2-His.

FIG. 12. ELISA measures the TG2-FN interaction. A, Design of tELISA measuring the TG2-FN interaction. B, Specificity is demonstrated by incubating His-tagged TG2 with increasing concentrations of biotinylated FN42 (from 0 nM to 16 nM) in the presence of unlabeled FN42 (from 0 nM to 16 nM). C, ELISA measures dose dependent inhibition of TG2-FN42 interaction by TG53. D, Saturation curves of FN42 in the presence of increasing concentrations of TG53 were used to calculate the Ki (4.15 μM) of TG53 for TG2. Inset corresponds to representative Lineweaver-Burk plots showing that TG53 competes for the same binding site in TG2 as FN42.

FIG. 13. Top SMIs inhibit cell adhesion to FN. A, Effects of top compounds (25 μM) on SKOV3 cells adhesion to FN. B, Dose-dependent effects (1-25 μM) of TG53 on SKOV3 cell adhesion to FN. C, Comparison between TG53 and a structurally similar compound (TG288) that does not inhibit TG2-FN interaction, on SKOV3 cells adhesion. D, Effects of TG53 (25 μM) on SKOV3 cells adhesion to wells coated with collagen-1 (20 μg/ml). E, Effects of TG53 (25 μM) on SKOV3-control or AS-TG2 cells adhesion to FN. Bars represent means+/−SEM of quadruplicate measurements. * denote p<0.05.

FIG. 14. Overall optimization plan to develop potent and drug-like TG2/FN inhibitors. Key elements include a robust iterative optimization process that closely integrates medicinal chemistry, in vitro screening, and ADME testing. Promising compounds will be used both as tool compounds to study the effects of TG2/FN inhibition, as well as drug development candidates.

FIG. 15. Chemical structure of the TG53 and of the 6 analogs targeting TG2-FN protein-protein interaction (MT1-MT6).

FIG. 16. Effects of TG53 and its analogues in ovarian cancer cells (OVC433 and SKOV3). A. Adhesion to FN was measured through a solid phase assay in a dose dependent experiment. B. Migration across a transwell coated with FN was measured after cells were pretreated with MT2-6 analogues (10 μM). C. Adhesion of OC cells to a layer of LP9 mesothelial cells during incubation with MT5 and MT6, at the indicated concentrations. D. and E. Number of GFP-labeled SKOV3 cells injected ip in nude mice with or without SMIs (D. MT4 and E. MT6) and recovered 2 hours later (data are presented as % of control). Bars represent means of quadruplicate experiments+/−SD. * p<0.05. *** p<00.001.

FIG. 17. Docked pose of TG53 bound to TG2. Amino acid side chains that interact with the inhibitor are labelled. A deep hydrophobic pocket extends into the left-hand side which can be exploited to create more potent inhibitors.

FIG. 18. (Top) Proposed synthetic route to access analogs of our TG2/FN inhibitor compound series. The efficient 3-step route will allow access to a wide variety of desired analogs. (Middle) Our lead compound TG53 is shown with selected physiochemical properties. A small set of proposed analogs are also shown which explore new chemical space, improve physiochemical properties, reduce liability for oxidation of the central ring, and increase IP. (Bottom) Structure of proposed biotinylated TG53 with a PEG linker for use in proteomics MS studies.

FIG. 19. TG2/FN/Integrin 01 complex regulates spheroids proliferation and tumor initiating capacity. A. Graphical representation of the epitope targeted by the 4G3 mAb overlapping with the FN-binding domain of TG2 (amino acids 1-165). B. Co-IP with anti-TG2 and anti-FN mAbs of cell lysates from OVCAR5 spheroids treated with 4G3 (10 μg/ml) for 6 days. Western blotting was performed by using anti-TG2 and FN monoclonal antibodies. C. Densitometric analysis results are shown as means±SEM. (N=3; *P<0.05, **P<0.01). D-F. CCK-8 assay quantifies proliferation of spheroids derived from OC cell lines and primary cells treated with inhibitory mAbs directed against the FN-binding domain of TG2 (4G3), and integrin β1 (clone P5D2) (N=8; *P<0.05, **P<0.01, ****P<0.0001). G-H. Tumor weights and volumes derived from ALDH+/CD133⁺ sorted from OVCAR5 cells and treated with 4G3 or IgG control and injected sq in nude mice, as described (N=5; **P<0.01). I. Time to tumor formation for 10,000 ALDH+/CD133⁺ cells pre-treated with IgG or 4G3, grown as spheroids for 6 days, and injected sq in nude mice. J. Spheroid morphology (left panel) and proliferation assay (right panel) of cells isolated from xenografts and grown ex vivo. (N=3; ***P<0.001).

FIG. 20. TG2 and Fzd7 directly interact in OCSCs. Co—IP with anti-TG2 and anti-FN mAbs of lysates from OVCAR5 spheroids treated with 4G3. Western blotting used anti-Fzd7, Fzd1, and GAPDH mAb. Densitometry quantification (N=3; ****p<0.0001).

FIG. 21. Proteomics analytical strategy

FIG. 22. TG53 tolerability in vivo. Three doses were administered 25, 50 and 100 mg/kg to cohorts of 3 mice. A) Average weights, B) average white blood cell counts (WBC), C) average hemoglobin (Hgb) and D) average platelet (Plt) counts measured 1 week after administration at the 3 doses (n=3).

FIG. 23. Half maximal inhibitory concentration (IC50) of inhibitors 2997, 2998, 3002, 3010, and 3011 for TG2-Fibronectic interaction.

FIG. 24. AlphaScreen Signal versus log[inhibitor], M for inhibitors 2997, 2998, 3002, 3010, and 3011.

FIG. 25. Percent (%) attached cells versus concentration of inhibitor (μM) for inhibitors 2997, 2998, 3002, 3010, and 3011.

FIG. 26. Percent (%) attached cells versus concentration of inhibitor (μM) for inhibitors 2999, 3003, 3015, 3000, and 3004.

FIG. 27. Percent (%) attached cells versus concentration of inhibitor (μM) for inhibitors 3001, 3005, 3006, 3007, 3008, and 3009.

FIG. 28. Percent (%) attached cells versus concentration of inhibitor (μM) for inhibitors 3012, 3013, 3014, and 3016.

FIG. 29. Enthalpy kinetics of MT-4-TG2 interaction as measured by Isothermal Titration Calorimetry (ITC). FIG. 29 shows the interaction between MT4 and TG2 examined by Isothermal Titration Calorimetry. A solution of 160 uM MT4 was titrated into 10 μM TG2. After corrections for the heats of dilution, the normalized enthalpy values were plotted against the cumulative MT4:TG2 molar ratio for each injection. The solid red line represents the best-fit curve for a single site model. The observed enthalpy of this interaction is ΔH=−20.5 kcal/mol and the equilibrium dissociation constant is 5.1 μM.

FIG. 30. Effects of TG-53 and MT-4 on TGase activity. FIG. 30 shows TGase activity measured by a colorimetric assay using purified TG2 in the presence of DMSO (control) or TG053 and MT-4 at the concentrations indicated.

FIG. 31. TIRF analysis of 01 integrin receptors distribution upon cell treatment with SMIs at the indicated concentrations and adhesion onto FN coated plates. FIG. 31 shows fields of view containing more than one cell per condition. The presented results correspond to images shown in FIG. 4D. Scale bar=20 μm.

FIG. 32. Flow cytometry analysis of integrin 31 plasma membrane expression. FIG. 32 shows flow cytometry measuring surface expression of integrin β1 in OVCAR 5 cells (A) and SKOV3 cells (B). OC cells were pre-incubated 1 h with serial dilutions of MT4 (as compared to TG53- and DMSO-treated cells), and left to attach for on either plastic (P) or fibronectin (FN) coated 6-well plates. Anti-integrin 31 antibodies (0.5 μg/mL) were used as control.

FIG. 33. Dose response effects induced by TG2-FN inhibitors on FAK phosphorylation. FIG. 33 shows western blotting for FAK phosphorylation at 2 hours after SKOV3 cell adhesion to FN in response to pre-treatment with increasing doses of MT-4, MT-5, and MT-6.

FIG. 34. Experimental design for in vivo assay testing the activity of MT4. A) shows the schematic drawing of the assay testing SKOV3 cells attachment to mice peritoneal walls (as described in Materials and Methods section) and B) shows phase contrast microscopy images of OC cells recovered from mice peritoneal cavity 2 h after injection in the presence of MT-4 or vehicle (DMSO).

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein refers to a subject in need of treatment for a disease or disorder associated with transglutaminase 2 (TG2) activity and/or expression, such a disease or disorder associated with and/or modulated by the interaction between TG2 and fibronectin. A subject in need thereof may include a subject having a cancer that is characterized by the dissemination of cancer cells via the interaction of TG2 and fibronectin. A subject in need thereof may include a subject having a cancer that is treated by administering a therapeutic agent that inhibits the interaction between TG2 and fibronectin, and/or that inhibits dissemination of cancer cells.

The term “subject” may be used interchangeably with the terms “individual” and “patient” and includes human and non-human mammalian subjects.

The disclosed compounds, pharmaceutical compositions, and methods may be utilized to treat diseases and disorders associated with TG2 activity and/or expression which may include, but are not limited to cell proliferative diseases and diseases and disorders such as cancers. Suitable cancers suitable for treatment by the disclosed compounds, pharmaceutical compositions, and methods may include, but are not limited to ovarian cancer, non-small cell lung cancer, colon cancer, cancer of the central nervous system, prostate cancer, and breast cancer.

The disclosed compounds may be utilized to modulate the biological activity of TG2, including modulating the interaction of TG2 and fibronectin. The term “modulate” should be interpreted broadly to include “inhibiting” TG2 activity and/or otherwise modulating TG2 activity.

New Chemical Entities

New chemical entities may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂-.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3-to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²) R³—, —C(O)N R² R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that inhibits the biological activity of tissue transglutaminase 2 (TG2) may be administered as a single compound or in combination with another compound inhibits the biological activity of TG2 or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, u-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of tissue transglutaminase 2 (TG2). As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of tissue transglutaminase 2 (TG2).

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

Inhibitors of Tissue Transglutaminase 2 (TG2) and Uses Thereof

Disclosed are compounds, pharmaceutical compositions comprising the compounds, and methods of using the compounds and pharmaceutical compositions for treating a subject in need thereof. The disclosed compounds inhibit the activity of tissue transglutaminase 2 (TG2). As such, the disclosed compounds and pharmaceutical compositions may be utilized in methods for treating a subject having or at risk for developing a disease or disorder that is associated with TG2 activity which may be cell proliferative diseases and disorders such as cancer.

In some embodiments, the compounds disclosed herein have a formula I:

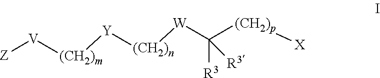

or a salt or solvate thereof,
wherein:
Z is pyrimidinyl (e.g., pyrimidin-2-yl) or 5,7-diazaindolinyl (e.g., 5,7-diazaindolin-6-yl) and Z optionally is substituted at one or more ring positions with alkyl (e.g., methyl), alkoxy (e.g., methoxy), amino, alkylamino (e.g., methylamino), dialkylamino (e.g., dimethylamino), heteroaryl (e.g., a nitrogen-containing heteroaryl group such as pyrrolidinyl and in particular N-pyrrolidinyl), or halo (e.g., chloro);
V is O or NR¹, wherein R¹ is hydrogen or alkyl (e.g., methyl);
m is selected from 0-6;
Y is an aryl group (e.g., phenyl), a diaryl group (e.g., diphenyl), or a heteroaryl group (e.g, pyridinyl such as pyridin-2-yl or pyridine-3-yl, or thiophenyl such as thiophen-2-yl or thiophen-5-yl), and Y optionally is substituted at one or more ring positions with haloalkyl (e.g., trifluoromethyl);
n is selected from 0-6;
W is CH or NR², wherein R² is hydrogen or alkyl (e.g., methyl);
R³ and R^{3′} are hydrogen or R³ and R^{3′} together form oxo;
p is selected from 0-6; and
X is an aryl group (e.g., phenyl or naphthyl such as naphth-1-yl) or a heteroaryl group (e.g, pyridinyl such as pyridin-2-yl or pyridin-3-yl or pyridine-4-yl, piperazinyl such as piperazin-1-yl, thiophenyl such as thiophen-2-yl, pyrazol such as pyrazol-1-yl, and indolyl such as indol-3-yl), and X optionally is substituted at one or more ring positions with one or more of alkyl (e.g., ethyl), alkoxy (e.g., methoxy), halo (e.g., chloro); amino; alkylamino (e.g., methylamino), or aryl (e.g., phenyl); and
optionally with the proviso that if m is 0, n is 0, and p is 1, then X is not N-imidazyl or indol-3-yl; and
optionally with the proviso that if m is 0, n is 0, and p is 1, then Z is not 4-dimethylamino-pyrimidin-2-yl or 4-pyrrolidin-1-yl-pyrimidin-2-yl.

In some embodiments of the disclosed compounds having formula I, the compounds have a formula Ia:

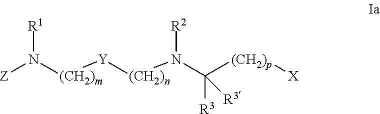

In some embodiments, the disclosed compounds having a formula I have a formula Ib:

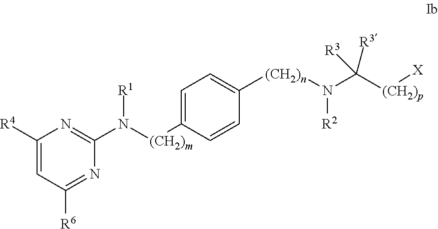

or a salt or solvate thereof,
wherein:
R⁴ is hydrogen or R⁴ has a formula,

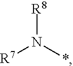

wherein R⁷ and R⁸ independently are hydrogen, alkyl (e.g. methyl), or a moiety comprising a biotinyl group or R⁷ and R⁸ together form linked alkyl (e.g., wherein R⁴ is a nitrogen-containing heterocycle); and
R⁶ is hydrogen or alkyl.

In some embodiments of the disclosed compounds, X has a formula selected from:

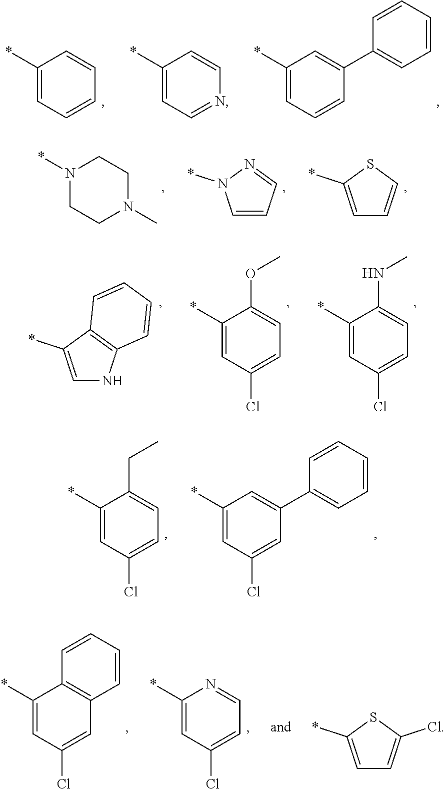

In some embodiments of the disclosed compounds, Y has a formula selected from:

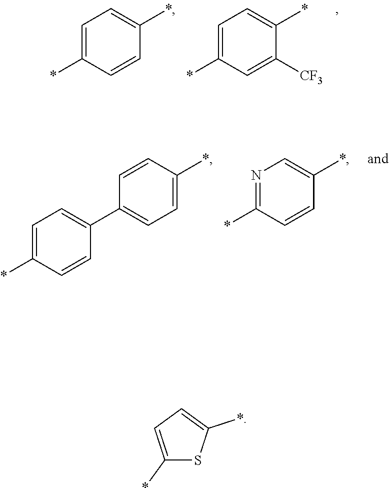

In some embodiments of the disclosed compounds, Z has a formula selected from::

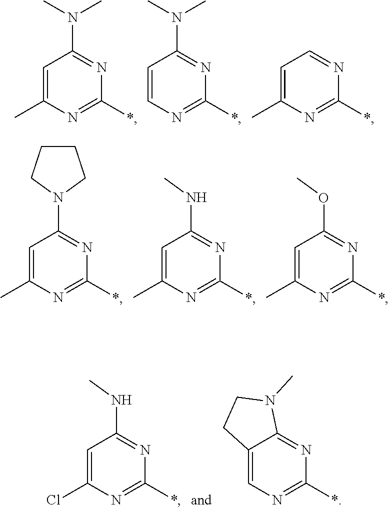

In some embodiments of the compounds having formula I, Ia, or Ib, R¹ is hydrogen. In other embodiments of the compounds having formula I or Ia, R¹ is methyl.

In some embodiments of the compounds having formula I, Ia, or Ib, R² is hydrogen. In other embodiments of the compounds having formula I or Ia, R² is methyl.

In some embodiments of the compounds having formula I, Ia, or Ib, R³ and R^{3′} together form oxo.

In some embodiments of the compounds having formula Ib, R⁴ has a formula,

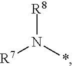

wherein R⁷ and R⁸ are hydrogen, methyl, or a moiety comprising a biotinyl group.

In some embodiments of the compounds having formula I, la, or Ib, X is a phenyl group optionally substituted at one or more ring positions with one or more alkyl, alkoxy and halo.

In some embodiments of the compounds having formula I, Ia, or Ib, m is 0 or 1; n is 0 or 1; and/or p is 0 or 1.

In some embodiments of the compounds having formula I, Ia, or Ib, R¹ is methyl, and R⁴ has a formula

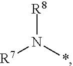

wherein R⁷ and R⁸ are methyl; and X is phenyl, optionally substituted with one or more alkyl, alkoxy and halo.

In some embodiments of the compounds having formula I, Ia, or Ib, the compound has a formula selected from:

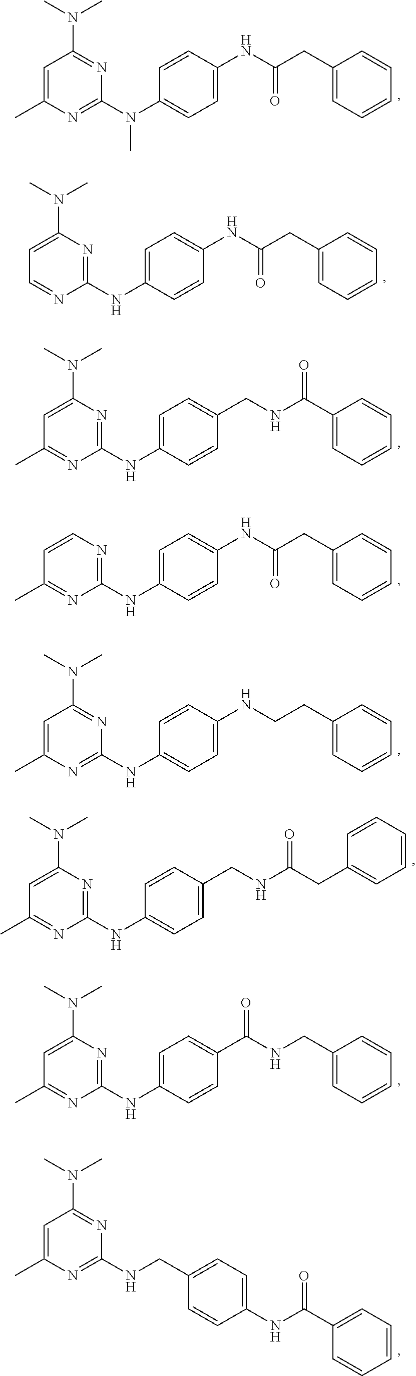

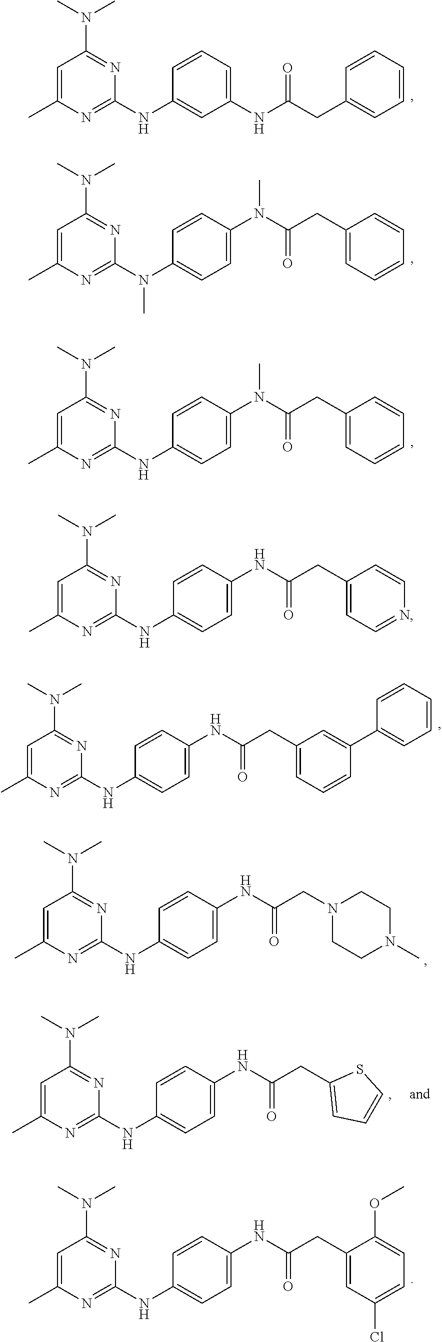

Also disclosed herein are pharmaceutical compositions comprising the compounds disclosed herein and a pharmaceutically acceptable carrier. The pharmaceutical compositions may be formulated for administration to a subject in need thereof, such as a subject having a disease or disorder that may be treated by administering a therapeutic agent that inhibits the biological activity of tissue transglutaminase 2 (TG2).

In some embodiments, the disclosed pharmaceutical compositions comprise a compound having a formula selected from:

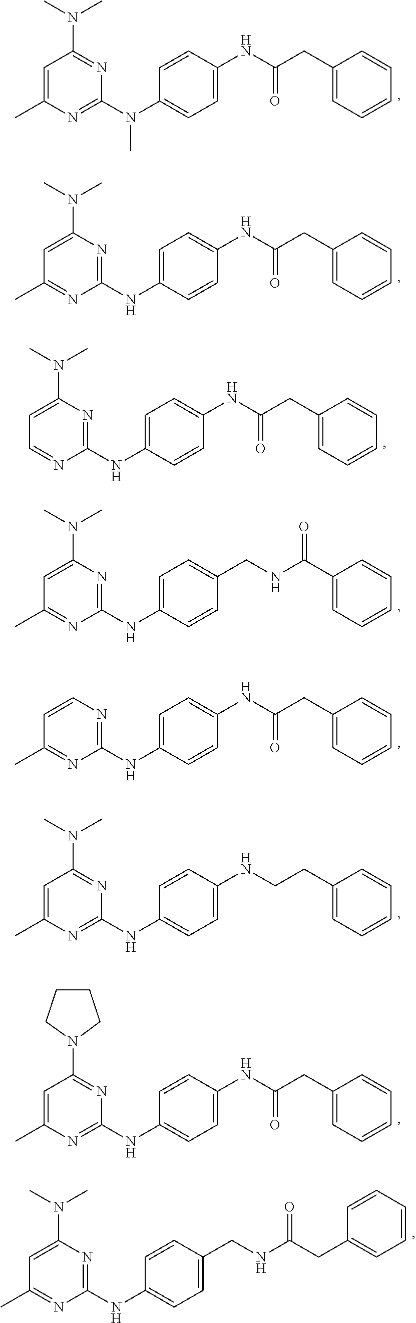

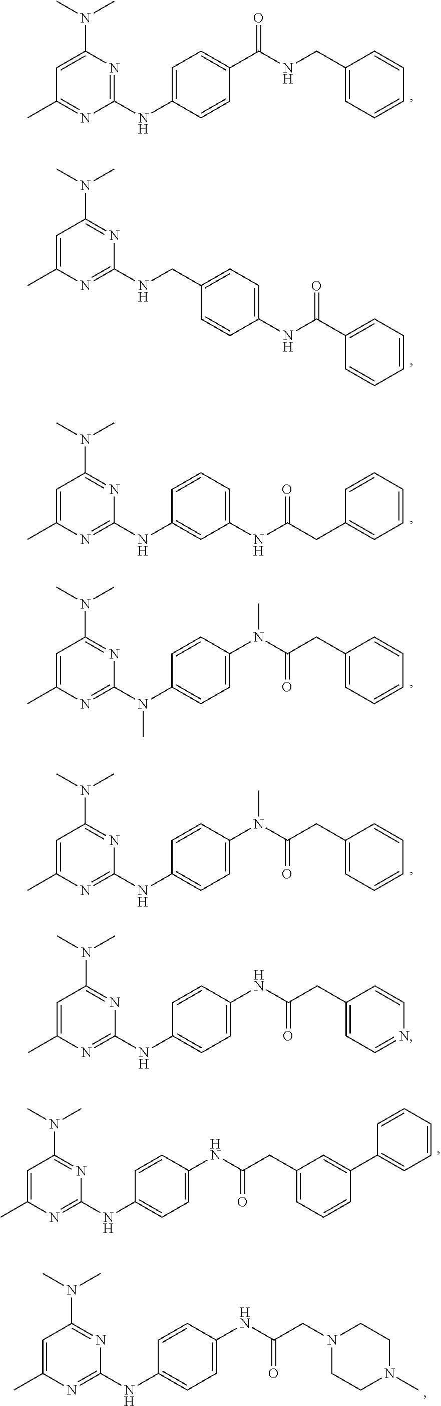

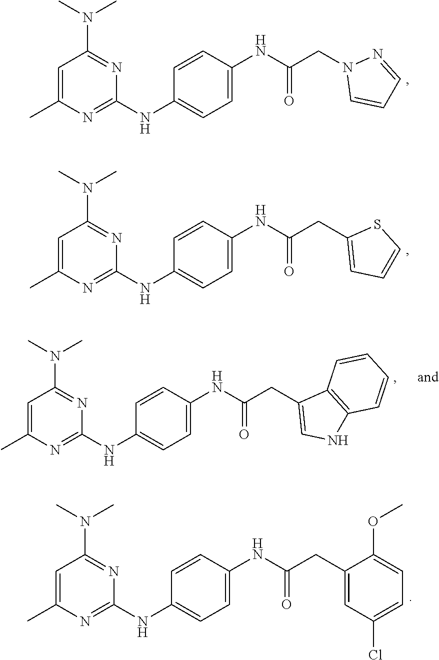

Also disclosed are methods of treating a subject in need thereof. In some embodiments, the disclosed methods include treating or preventing a disease or disorder associated with tissue transglutaminase in a subject in need thereof, the method comprising administering to the subject any of the compounds disclosed herein and/or a pharmaceutical composition comprising any of the compounds disclosed herein. Diseases or disorders treated by the disclosed methods may include cell proliferation diseases or disorder. Cell proliferation disease or disorders may include cancers such as ovarian cancer.

### EXAMPLES

The following Examples are illustrative and are not intended to limit the scope of the claimed subject matter.

### Example 1—Small Molecules Target the Interaction Between Tissue Transglutaminase and Fibronectin

Reference is made to Sima et al., “Small Molecules Target the Interaction between Tissue Transglutaminase and Fibronectin,” Molec. Canc. Thera. 2019; 18:1057-78, published Apr. 23, 2019, which content is incorporated herein by reference in its entirety.

Interactions between cancer cells and the tumor niche occur early during tumorigenesis and promote cell survival, evasion from chemotherapy, and establishment of metastatic sites. In ovarian cancer (OC), contact with the fibronectin (FN) and collagen-rich mesothelial matrix critically regulates the steps of peritoneal dissemination(1). We identified tissue transglutaminase (TG2), a protein which anchors epithelial cells into the extracellular matrix (ECM) through interactions with FN and integrins(2), as being highly expressed in ovarian tumors at the interface between cancer cells and the tumor microenvironment (TME) (3). We showed that when injected intraperitoneally (ip) or under the ovarian bursa of nude mice, OC cells engineered to express decreased levels of TG2 disseminated less efficiently, as compared to control cells(3-5).

The process of metastasis in solid tumors requires loss of cell-cell contact and breakdown of the basement membrane followed by tumor invasion into lymphatic or vascular channels(6). By contrast, OC harbors a distinct pattern of peritoneal metastasis, with hematogenous dissemination being uncommon(1). OC cells are in direct contact with the overlying peritoneal surface and fluid and simple dislodgement from the primary tumor allows cells to float in the peritoneal space, where they adhere and form metastatic implants. In the peritoneal fluid, OC cells aggregate as spheroids, which provide protection from the stress imposed by the extracellular environment. Interactions between cancer cells and the peritoneal mesothelium activate “outside-in” signaling (7) which stimulates cell proliferation, survival and tumor angiogenesis. Over the past decade, our group demonstrated sequentially that TG2 regulates EMT (4), migration of cells from the primary site(3), formation of spheroids in the peritoneal fluid (8), and invasion into the peritoneum (3, 9). These steps regulated by the protein through its interaction with FN contribute to establishment of peritoneal implants (3, 4) and render TG2 an attractive new cancer target.

TG2 is a multifunctional protein, which catalyzes Ca²⁺-dependent post-translational protein modifications and has a well-defined binding site for FN. Our mechanistic studies identified the interaction between TG2 and FN as being a critical player in the process of intraperitoneal (ip) dissemination. We demonstrated that the TG2/FN complex is implicated in OC metastasis via multiple mechanisms including: adhesion to the ECM by strengthening integrin-dependent cell-matrix adhesion (3), induction of epithelial to mesenchymal transition (EMT) (4, 8), regulation of Wnt/β-catenin signaling (10), through direct interaction with the Frizzled 7 (Fzd7) receptor, which in turn drives OC cell proliferation and persistence of a stem cell profile (5), remodeling of the extracellular matrix (11), and fine tuning of intracellular oncogenic signaling (10, 12). An antibody that disrupted TG2/FN complexes inhibited cancer stemness characteristics, spheroid formation, and tumor initiation (5).

Based on these results, we hypothesized that the TG2/FN interaction is targetable and that its disruption by small molecules will prevent cancer cell adhesion to the matrix and OC metastasis. To this end, we completed a high throughput screening (HTS) campaign of compounds in the ChemDiv library by using a newly developed AlphaLISA assay that robustly measured the TG2/FN complex formation and identified a new class of potent inhibitors for this protein-protein interaction (PPI) (13). We showed that several of the small molecules discovered through the screen potently blocked OC cell adhesion and migration, demonstrating proof-of-principle for blocking this protein complex to diminish cancer cell invasiveness and perhaps peritoneal dissemination. The best hit (TG53) exhibited good biochemical potency and had highly efficacious cellular activity. Given its promising properties, we used TG53 as a starting point to develop more potent and selective TG2/FN inhibitors by using rational medicinal chemistry optimization. Here we show that newly synthesized analogues possess improved in vitro and in vivo efficacy in OC models. This new series of TG2/FN inhibitors potently blocks cellular adhesion to FN and to a reconstituted peritoneal matrix, resulting in inhibition of “outside-in” signaling and sensitization of cancer cells to paclitaxel. Our results identify new small molecules targeting the TG2/FN complex and the initial steps of cellular adhesion for future preclinical development.

Materials and Methods

Chemicals and Reagents

Unless stated otherwise, chemicals and reagents were from Sigma-Aldrich (St Louis, Mo., USA). Anti-integrin 31 antibody was from Chemicon (Cambridge, Mass., USA); PE anti-CD29/integrin 31 (#303004), from BioLegend (San Diego, Calif., USA); anti-pERKI1/2(#9101), anti-ERK (#9102), anti-pFAK (#3283), and anti-FAK (#3285), from Cell Signaling (Beverly, Mass., USA); anti-vinculin (# ab18058), from Abcam(Cambridge, Mass., USA); anti-pFAK(#44-625G) used for IF was from Thermo Scientific (Fremont, Calif., USA); and anti-GAPDH from Biodesign International (Saco, Me., USA). Antibodies for phospho-Src (Tyr416), phospho-FAK (Tyr576/577), and c-Src used for confocal imaging were from Cell Signaling Technology, Inc. (Beverly, Mass., USA), monoclonal TG2 (CUB 7402) was from Thermo Fisher Scientific, integrin β1 was from EMD Millipore (Billerica, Mass., USA).Secondary HRP-conjugated antibodies were from Amersham Biosciences (San Francisco, Calif., USA) and Santa Cruz Biotechnology Inc (Santa Cruz, Calif., USA).

Cell Culture

SKOV3 cells were obtained from the American Type Culture Collection (ATCC, Manassas, Va.), and cultured in growth media containing 1:1 MCDB 105 (Sigma, St. Louis, Mo.) and M199 (Cellgro, Manassas, Va.).OVCAR5. SKOV3-GFP, and OVCAR433 were provided by Drs. Peter (Northwestern University, Chicago, Ill.) and Bigbsy (Indiana University, Indianapolis, Ind.), respectively, and were grown in RPMI media (Corning). LP9, a mesothelial cell line, was procured from the Coriell Institute (Camden, N.J., USA) and grown in M199/MCDB 151 medium with 15% FBS and 0.4 μg/ml hydrocortisone. Media was supplemented with 10% FBS and 1% Penicillin-Streptomycin. Cells were cultured at 37° C. in a humidified incubator with 5% CO2. All cells were authenticated and tested negative for Mycoplasma at least yearly. Cells were passaged no more than 15 times after thawing.

Design and Synthesis of TG-53 Analogues.

The synthetic schemes and detailed experimental procedures are included in Supplemental Information. All six compounds MT-1-6 were synthesized from a common intermediate N2-(4-aminophenyl)-N4,N4,6-trimethylpyrimidine-2,4-diamine (compound S1, structure shown in Supplemental Material). Compound S1 was prepared from commercially available compounds crimidine and p-phenylenediamine. A solution of crimidine and p-phenylenediamine (1:1 mole ratio) in dimethyl formamide (DMF), were sealed in a glass microwave reactor and radiated with microwave to heat up to 150° C. and maintained for 15 min. After cooling to room temperature, the solid product was collected by filtration, and washed with ethyl acetate to give compound S1. Compound S1 was then condensed with different carboxylic acids in DMF, using propyl phosphonic anhydride (T3P) as the coupling reagent to give the desired products (MT-1-6). Compounds were purified by reversed-phase HPLC and purity was confirmed with LC-MS and NMR (Figures S1-S6).

Bio-Layer Interferometry (BLI) and Isothermal Titration Calorimetry (ITC)

The 45 kDa fibronectin fragment (FN45) corresponding to the TG2 binding region (Sigma, # F0162) was biotinylated using Biotin (Type A) Fast Conjugation Kit (Abcam, # ab201795). Biotinylated FN45 (10 μg/mL in PBS) was captured on pre-hydrated streptavidin-coated sensors (Pall Forte Bio SAX 18-5117). At each TG2 concentration, the binding kinetics were determined by following this sequence run: baseline (PBS for 30 s), association step (varying concentrations of TG2 in PBS for 120 s), and dissociation step (PBS for 120 s). A new FN45 biosensor was prepared for each run. To analyze the effect of the inhibitor MT4 on the interaction between TG2 and FN, a solution of 20 μg/mL of bio-FN45 was used for capturing FN45 on the streptavidin-coated sensors and the association step was 90 s. For each sensor, the capturing step was measured in real-time in order to assess the stability of the signal and the reproducibility between sensors. The saturation level Rmax was 0.6 (+0.08) for all sensors and no dissociation of the biotinylated FN45 was observed over 60 minutes. Control experiments performed with bare sensors (without FN45) showed no significant binding of TG2. For the experiments with (TG2+MT4), controls were performed at each MT4 concentration and Rmax values were corrected for non-specific binding and any effects of DMSO on the TG2-FN45 interaction (these effects were below 8%). All BLI experiments were performed at 22° C. Data fitted globally using the BLItz Pro 1.10.31 software package as described(14, 15). Isothermal titration calorimetry (ITC) was performed at 25° C. by titrating 160 μM MT4 into a 10 μM solution of TG2 (Sigma, #T5398).

Cell Adhesion to LP9 μMesothelial Cells Monolayers

LP9 mesothelial cells added to clear-bottom 96-well plate at 12,000 cells/well were grown to confluency over 48 hours (h). OC cells were labeled with calcein AM and seeded at a density of 4×10⁴ cells to monolayers of mesothelial cells and incubated for 1 h. Non-adherent cells were removed by washing with PBS.

Total Internal Reflection Fluorescence (TIRF) Microscopy

For assessing the potential effect of SMIs on 01 integrin distribution at the interface between cells and the FN matrix, OVCAR5 cells were treated for 1 h with either TG53 or MT-4 in serum-free media at 37° C., and then seeded onto FN-coated glass bottom microwell dishes (MatTek Corp. Ashland, Mass.) for 45 minutes at 37° C. Samples were fixed in PFA and blocked in 1% BSA. 01 integrin receptors were detected by using anti-01 integrin antibodies (clone P5D2, Chemicon, #MAB1959, 1:100) and AlexaFluor 568-conjugated anti-mouse secondary antibodies, while actin was labeled using Alexa Fluor 488-conjugated phalloidin. Cells were imaged with a Nikon Ti2 Microscope equipped with a Photometrics Prime 95B camera using a Nikon Plan Apo 100×1.49 NA TIRF objective.

In Vivo Cell Attachment

All animal experiments were conducted in accordance with the recommendations for the Care and Use of Laboratory Animals of the National Institutes of Health under a protocol approved by Indiana University Institutional Animal Care and Use Committee. 5×10⁶ SKOV3-GFP cells diluted in 100 μl were injected intraperitoneally (ip) along with compound MT4 or MT6 (10 μg/kg) in six-week-old female NOD-SCID gamma (NSG) mice (Jackson laboratories). After 2 hours, mice were euthanized, and the peritoneal cavity was washed thoroughly with 4 mL PBS. The peritoneal fluid containing cells that had not adhered to the mesothelial layer was gently collected and cell number was determined by using the Luna FL Cell Counter (Logos Biosystems). Ten replicates per group were assessed.

Enzyme-linked Immunosorbent Assay (ELISA); TG2 enzymatic activity assay; CCK-8 cell proliferation assays; Solid phase adhesion assay; Solubility assay, Wound-healing assay; Early attachment assay; Immunofluorescent (IF) staining and confocal microscopy; Western blotting; Colony and sphere forming assays, see Supplementary material (SM) and published methods (5, 13, 16).

Statistical Analysis

GraphPad 7.04 Software (Prism, San Diego, Calif., USA) was used for all statistical analyses. The Student's t test was used to test statistical significance between groups.

Results

Design and Synthesis of New SMIs Targeting TG2-FN Interaction

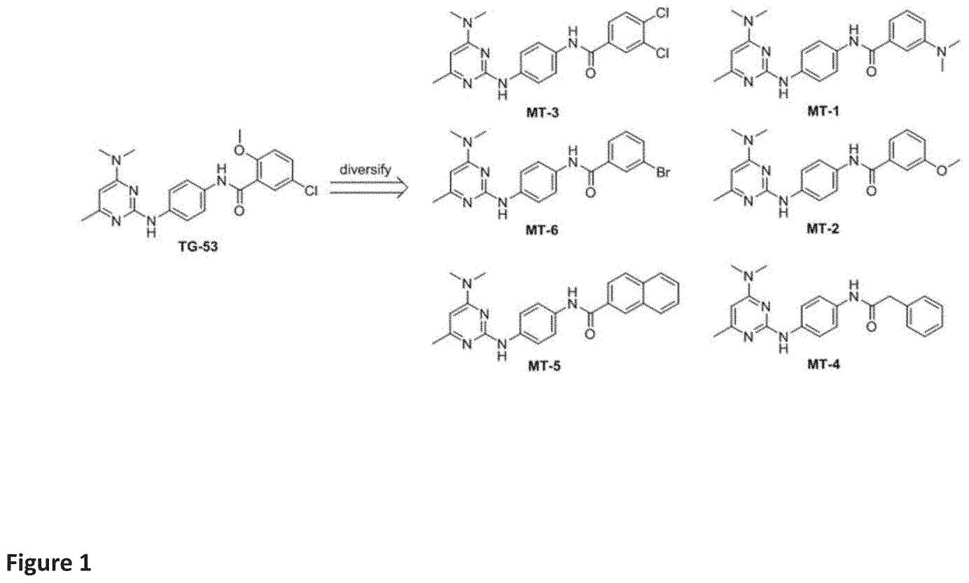

Six new compounds were designed based on the chemical structure of the previously reported diaminopyrimidine derivative TG53 (13). The structure of TG-53 is shown in FIG. 1. Hit optimization strategy was focused on the improvement of TG-53 potency and solubility. A small group of derivatives were designed to diversify the carboxylic amide group of TG-53 (FIG. 1). Compounds MT-3, MT-5, and MT-6 were designed to increase the binding activity by enhancing the potential interaction between the molecule and the target protein. MT-3 adds an additional chloride group at the p-position; MT-6 substitutes the original chlorine atom with a larger bromo group; and MT-5 replaces the benzene ring with a larger naphthalene ring. Compounds MT-1, MT-2 and MT-4 were designed to increase the solubility. MT-1 installs an amine group to replace the chloride, which should improve compound solubility under acidic conditions; MT-2 uses a methoxyl group to replace the chloride, which should improve solubility at neutral pH; and MT-4 adds an additional methylene group between the two aromatic groups (FIG. 1), which increases the flexibility of the whole molecule, and thus should increase the overall solubility under all conditions. The measured solubility of MT4 was 91.5 μM compared to TG53 (<5 μM; see SM).

In Vitro Inhibition of TG2/FN by MT1-6 Compounds

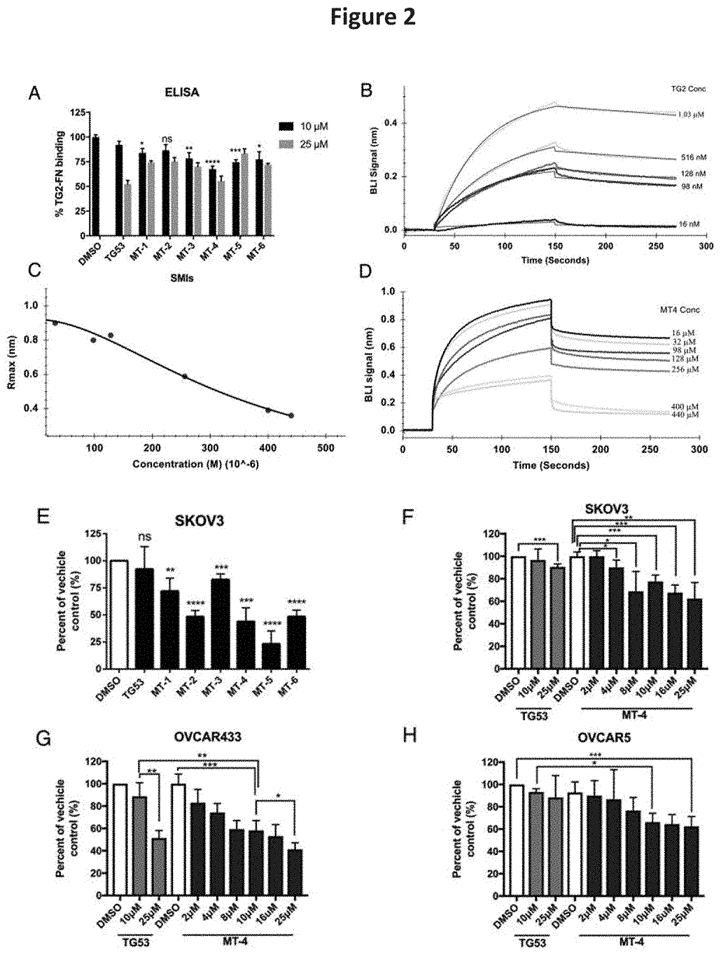

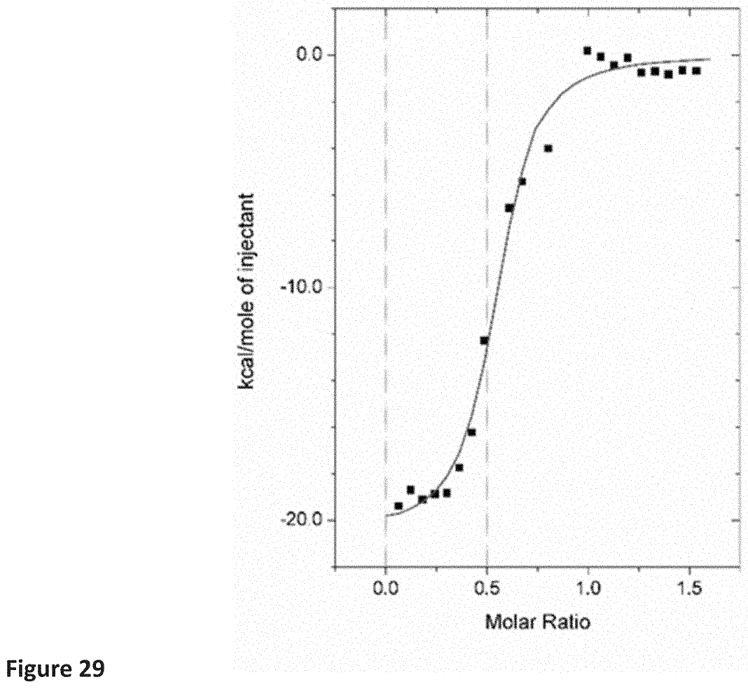

The effects of MT1-MT6 on the interaction between rTG2 and FN42 were measured first by ELISA (13). MT-3, MT-4, MT-5, and MT-6 inhibited the interaction slightly more potently than TG53 at 10 μM and similar to TG53 at 25 μM (FIG. 2A). Next, BLI was employed to determine to kinetics of the TG2/FN interaction. As reported previously (17), TG2 and FN interact with high affinity. In our assay, the association and dissociation kinetics were: kₐ=5320/Ms, kₐ=0.0016/s (FIG. 2B) and the equilibrium affinity (Kd) was 0.30 nM. To examine the effect of MT4 on the formation of the rTG2-FN45 complex, first, FN45 was first captured on BLI sensors and incubated with MT4 in concentrations ranging from 16 nM to 500 μM for 15, 30, and 45 minutes. In these experiments, FN45 and rTG2 complex formation was not affected by MT4. Second, rTG2 was incubated with MT4, prior to BLI. Dose-dependent inhibition of the association between FN45 and rTG2 was observed (FIG. 2C). The interaction decreased with increasing MT4 concentrations, and a faster dissociation was observed in the presence of MT4 (FIG. 2D), suggesting that MT4 binds to TG2 and disrupts the binding interface with FN. To further examine this possibility, calorimetric titrations of MT4 into rTG2 were performed. The observed change in enthalpy for this interaction, ΔH, was −20.5 kcal/mol and the apparent equilibrium dissociation constant, K_{d}, was 5.1 μM (FIG. 29), supporting that MT4 interacts with TG2.

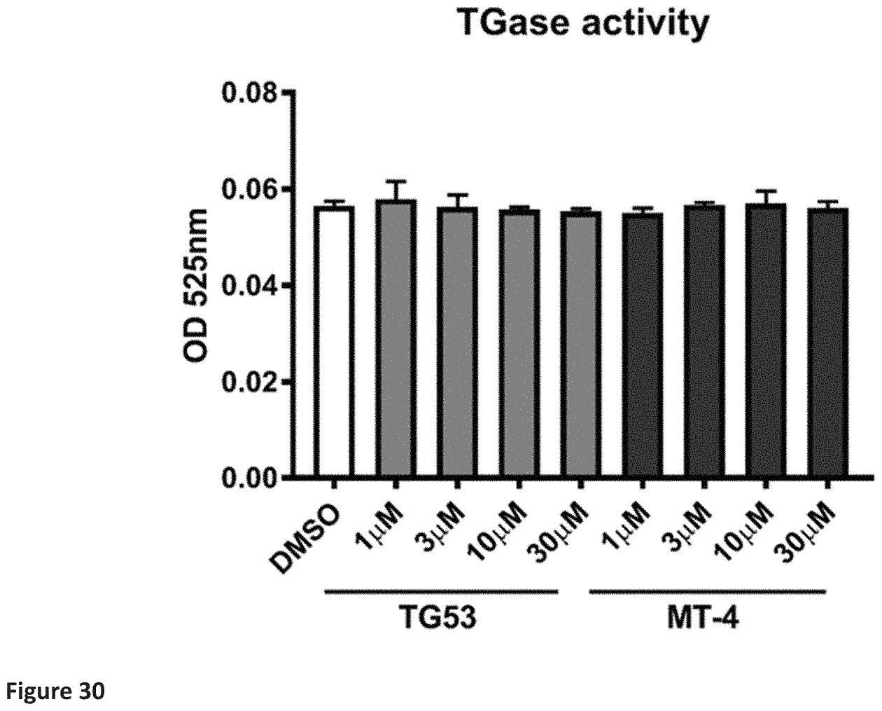

Next, the effects of TG53 and analogues on the enzymatic function of TG2 were measured by using a TGase colorimetric assay (18). The SMIs did not interfere with the enzymatic activity of TG2 (FIG. 30), consistent with the fact that the enzymatic core of the protein (Cys²⁷⁷) is distinct from the FN-binding site (aa⁸⁸⁻¹⁰⁶)(19).

Effects of MT1-6 on Cell Adhesion:

To measure whether disruption of the TG2-FN complex interferes with cellular adhesion to the ECM, a solid phase assay quantified OC cell attachment onto FN in the presence of SMIs or DMSO. MT2, MT4, MT5, and MT6 potently blocked OC cells' adhesion to FN (FIG. 2E, *, p<0.05; **, p<0.01). Because MT-4 was active and the most soluble (see SM), we characterized its effects in-depth, as a representative of this series. Solid phase assays performed with SKOV3 (FIG. 2F), OVCAR433 (FIG. 2G) and OVCAR5 (FIG. 2H) demonstrated dose-dependent inhibitory effect of MT-4 on OC cell adhesion to FN. MT-4 was more active than TG53 at 10M (**, p<0.01, FIG. 2G-H).

SMIs Prevent the Formation of Stable Cell Contacts with the FN

It has been proposed that TG2 facilitates formation of a “bridge” connecting integrin β1 with FN in the ECM (2). TG2/FN/integrin complexes are known to activate downstream signaling. Among the key proteins engaged after cell adhesion and spreading are the focal adhesion kinase (FAK) and the kinase c-Src, which is required for rapid actin and F-actin reorganization after early spreading (20). Therefore, we next investigated how inhibition of TG2-FN interaction by TG-53 and related inhibitors affects integrin clustering and downstream signaling, by using both immunofluorescent (IF) staining and western blotting.

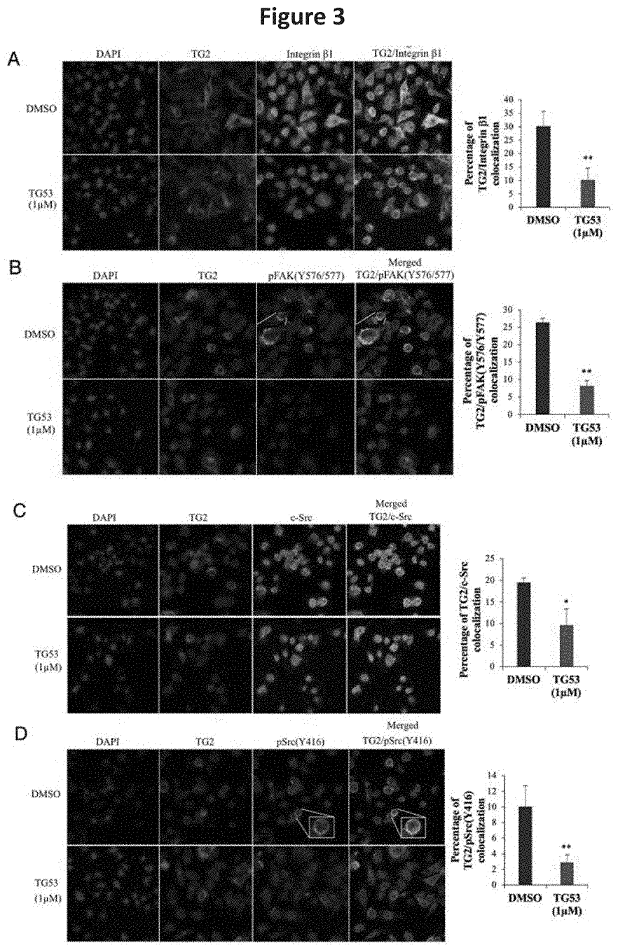

First, the interaction between TG2 with proteins in the integrin β1 adhesion complex was measured by confocal IF analysis of SKOV3 cells, in the presence or absence of 1 μM TG53 (FIG. 3A-D). A significant decrease in colocalization of TG2 with integrin 01 (FIG. 3A), phosphorylated FAK (pFAK) (FIG. 3B), c-Src (FIG. 3C), and phosphorylated Src (pSrc) (FIG. 3D) was recorded. Moreover, a marked decrease in FAK and Src phosphorylation was observed downstream of integrin 31 upon treatment with TG53 (FIGS. 3B and D).

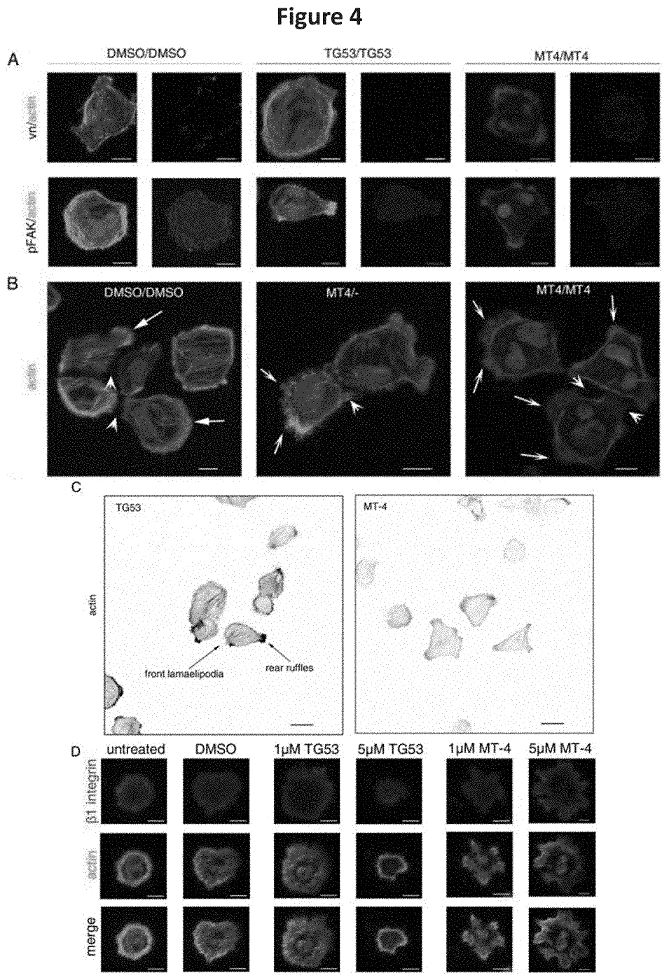

Next, we sought to dissect the mechanism by which TG53 and related SMIs interfere with cell attachment. For this, we performed an in vitro early cell attachment assay using OVCAR5 cells pre-treated with TG53 or MT4 (1 μM) for 72 hours. Cells were seeded for 30 minutes in serum-free media onto FN coated cover slips. IF staining for vinculin showed that exposure of cells to both MT-4 and TG53 before and during cell attachment decreased the number of focal contacts established with FN-coated substrate in comparison with DMSO treated controls (FIG. 4A, upper panel). While the vehicle treated cells established maturing focal adhesions (elongated shape, FIG. 4A, DMSO/DMSO, inset), cells treated with SMIs exhibited immature contacts (punctate shape, FIG. 4A, TG53/TG53, MT-4/MT-4, insets). Increased vinculin staining around the nucleus as compared to cell periphery was observed in cells treated with MT4, suggesting a lack of productive engagement of cell protrusions to bind FN. Both TG-53 and MT4 caused decreased FAK phosphorylation (FIG. 4A, bottom panel). Staining of actin filaments with phalloidin revealed generation of thin cell protrusions towards the FN surface in DMSO treated cells (FIG. 4B, left panel). However, in cells pre-treated with MT-4, although these protrusions were formed, they could not establish durable connections with the FN substrate. As a consequence of mechanic tension, they snapped back forming curly ruffles (FIG. 4B, middle panel, arrows). Also, cells lost polarity after exposure to MT-4 prior and during attachment, as evidenced by the formation of multiple lamellipodia (FIG. 4B, right panel, arrows). The absence of a single leading-edge characteristic of directional cell migration is clearer in high-contrast grayscale images illustrating the actin cytoskeleton affected by MT-4 treatment (FIG. 4C, right panel) versus TG53 (FIG. 4C, left panel). MT-4 treated cells displayed cortical localization of actin filaments, as opposed to TG53-treated cells, where parallel intracellular actin filaments are still present. This provides evidence that MT-4 affects actin cytoskeleton remodeling. Moreover, MT-4 affected cell-ECM adhesion, while the cell-cell interactions appear unaffected (FIG. 4B, middle and right panel versus left panel, arrowheads).

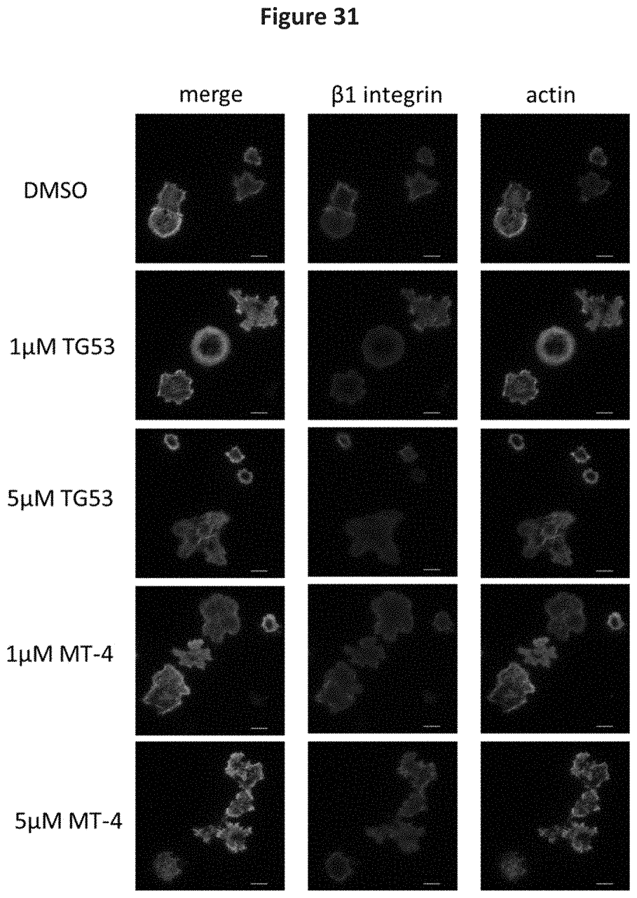

Having observed these effects exerted by SMIs on cell adhesion and based on previous knowledge that cell surface TG2 promotes integrin clustering (21), we next evaluated how targeting TG2-FN interaction impacts integrins' organization at the interface with FN. TIRF microscopy, which allows high resolution scanning at the cell-glass substrate interface through an evanescent light wave effect (22, 23) demonstrated that 31 integrins distribute evenly after OVCAR5 cells' attachment to FN-coated substrate (FIG. 4D, top panels; FIG. 31). DMSO (control) did not affect this pattern. Actin formed dense symmetrically distributed filaments that support the cell shape during cellular adhesion (FIG. 4D, lower panels; FIG. 31). Pre-treatment with 1 μM or 5 μM TG53 caused modest displacement of integrin receptors towards the cell perimeter and a disorganization of the actin cytoskeleton that form shorter spikes instead of long stretched filaments. Noteworthy, pre-incubation of OVCAR5 cells with 1 μM or 5 μM MT-4 caused enhanced 31 integrin transposition towards the extreme edge of the cell concurrent with cortical positioning of actin fibers (FIG. 4A-C; FIG. 31).

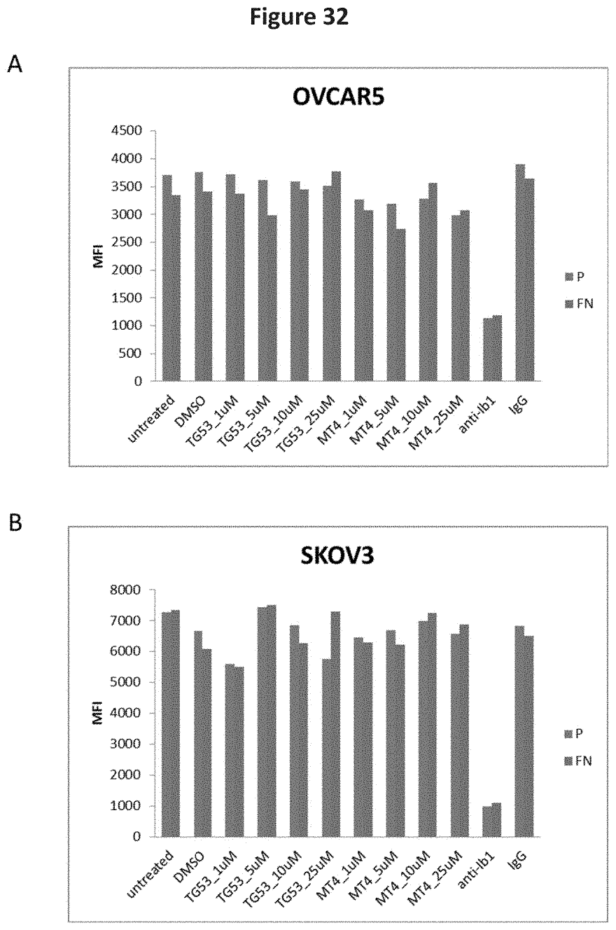

As a control, we used flow cytometry (FACS) to measure β1 integrin on the surface of cells treated with the SMIs. FACS did not show significant downregulation of β1 integrin receptors at the plasma membrane in SKOV3 and OVCAR5 cells treated with serial concentrations of TG53 and MT4, as compared to vehicle treated cells (FIG. 32). As controls, an anti-integrin β1 function blocking antibody significantly decreased β1 integrin expression, while a non-specific IgG control did not cause any changes. Altogether, TIRF and FACS analyses indicate that the main effect of the SMIs is redistribution of 1 integrin receptors towards the cortical region, with only minor changes observed at the plasma membrane (FIG. 4D and S10).

SMIs Attenuate Signaling Downstream of Integrin β1

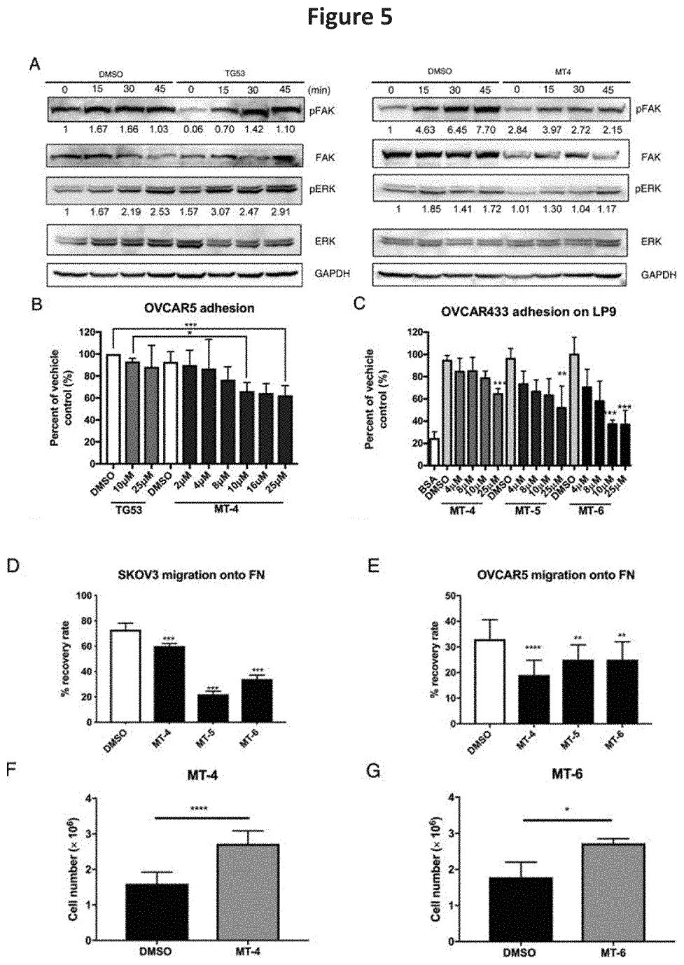

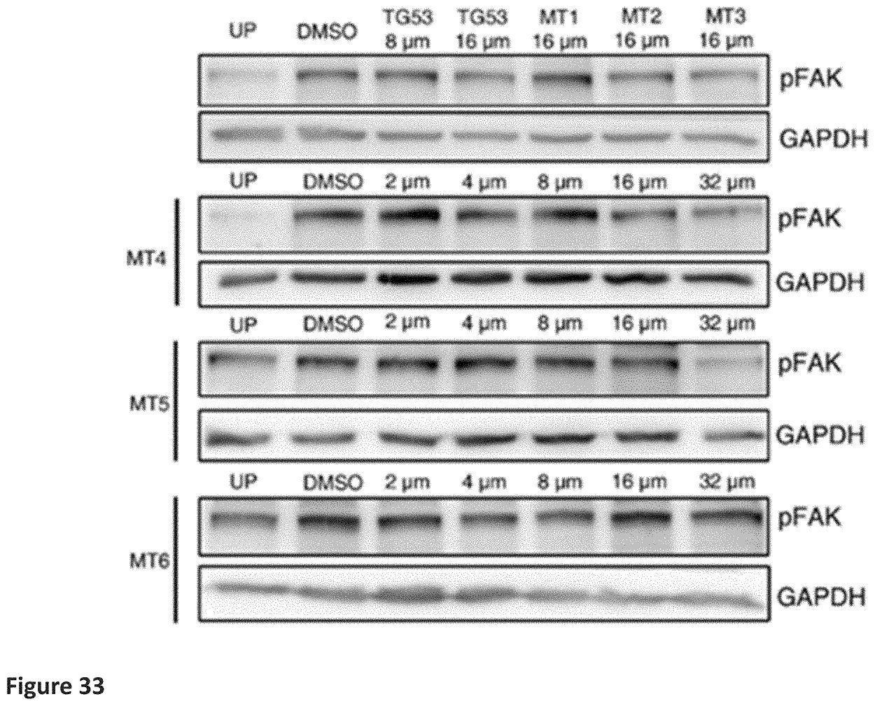

The effects of selected SMIs on “outside in” signaling activated in response to cell adhesion were also measured by Western blotting. Treatment of OC cells with TG53 (8 to 16 μM) attenuated FAK phosphorylation (FIG. 33). Similarly, MT-4 and MT-5 caused dose-dependent inhibition of FAK phosphorylation (FIG. 33). A time course experiment compared the effects of MT4 and of the parent compound TG53, on signaling activated in response to OVCAR5 cell attachment (FIG. 5A). FAK phosphorylation was modestly delayed in the presence of TG53, with a peak in activation at 30 minutes post cell seeding on FN as compared to 15 minutes for control (FIG. 5A, left). This inhibition was more evident after treatment with MT-4 (FIG. 5A, right), consistent with the results of the IF analysis. ERK phosphorylation was also inhibited and delayed in cells treated with MT-4 as compared to control or TG53-treated cells (FIG. 5A).

SMIs Prevented Attachment of OC Cells to a Mesothelial Cell Layer In Vitro and to Peritoneum In Vivo

As cellular adhesion has a potential impact on OC metastasis (24), the new SMIs could be developed as inhibitors of peritoneal dissemination. Based on results of previous analyses, MT4, MT5, and MT6 were selected for further characterization. First, cell adhesion to a mesothelial layer resembling the peritoneal cavity derived from LP9 normal primary mesothelial cells (25, 26) was assessed. Calcein-labeled OC cells were allowed to adhere to LP9 confluent monolayers in the presence of the SMIs or vehicle control. MT-4 and MT-5 significantly decreased SKOV3 cell binding to the mesothelial layer (FIG. 5B, ***p<0.001, ****p<0.0001). MT4, MT5, MT6 exerted a dose-dependent inhibitory effect on OVCAR433 adhesion to the LP9 layer, at concentrations >10 μM (FIG. 5C, **p<0.01, ***p<0.001).

After cell adhesion to the mesothelium, the next step in the process of peritoneal metastasis, is migration and invasion into the matrix. Thus, effects of SMIs on OC cell migration on FN coated substrates were also determined. MT4, MT5 and MT6 (8 μM), inhibited the motility of SKOV3 and OVCAR5 cells as measured by the ability to repair a scratch produced in the monolayer (FIG. 5D-E, **p<0.01, *** p<0.001, ****p<0.0001).

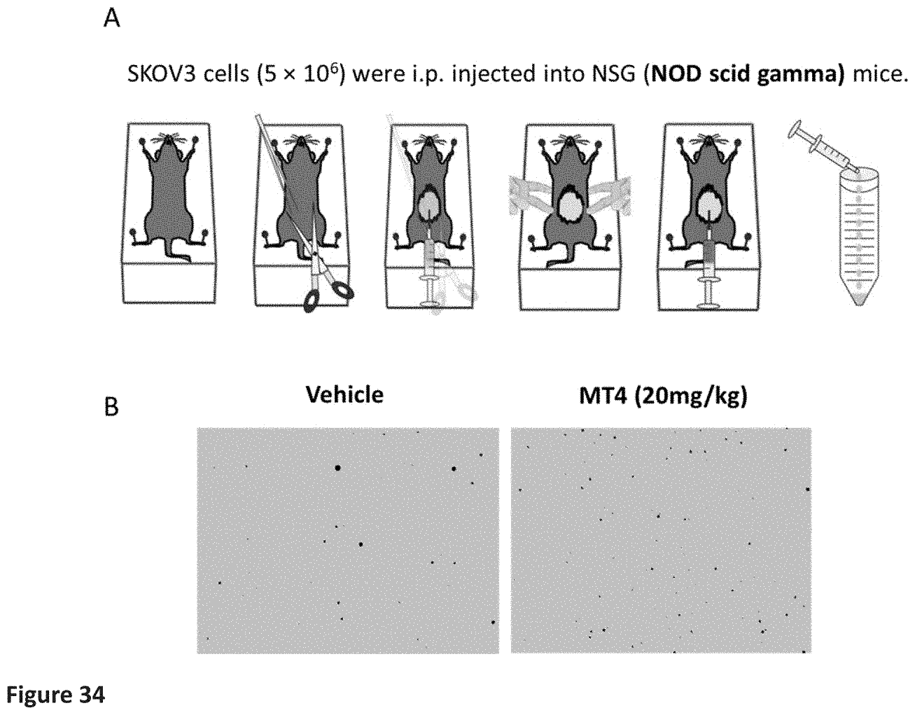

Lastly, the effects of SMIs on adhesion to the peritoneum were assessed in vivo. Stably expressing GFP SKOV3 cells pre-incubated with MT4 or MT-6 for 1 hour were injected ip. Two hours after injection, unattached cells were recovered from the peritoneal cavity and counted (FIG. 34). Increased numbers of OC cells were recovered from mice injected with MT4 or MT6, as compared to vehicle (FIG. 5F, G). MT-4 was more potent (p<0.0001) than MT-6 (p<0.05) preventing attachment of SKOV3 cells to the peritoneal cavity. These data support further development of this class of compounds to prevent peritoneal dissemination.

Anti-Proliferative Effects:

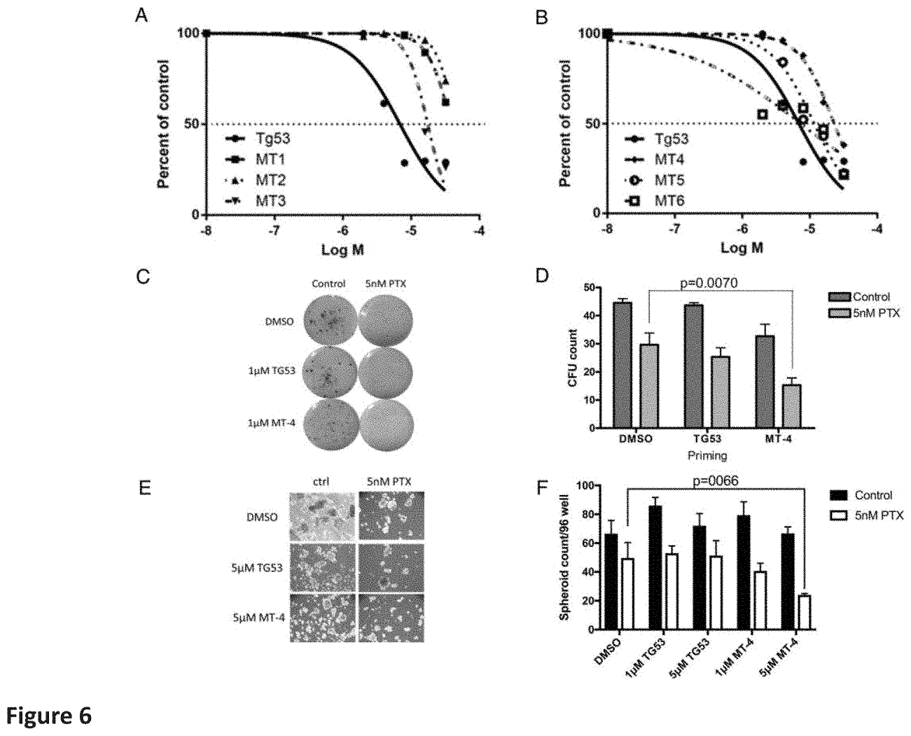

The parent compound TG53 was not cytotoxic (13). Similarly, the analogues MT1-6 did not exert anti-proliferative effects in SKOV3 cells during a 72-hour treatment period (FIG. 6A-B). We next considered potential combinations of the proposed SMIs with cytotoxic drugs and tested whether the compounds potentiated the effects of paclitaxel (PTX). Priming with 1 μM MT4 before PTX diminished colony formation compared to control (DMSO) or TG53-primed cells (FIG. 6C-D). Similarly, the combination of MT-4 and PTX inhibited OVCAR5 cell proliferation as spheres more potently than PTX alone or PTX and TG53 (FIG. 6E-F). However, priming of OVCAR-5 cells with TG2-FN inhibitors did not interfere with cell cycle progression or induced apoptosis as evidenced by Annexin V/7-AAD double staining. These results support that priming of OC cells with TG2-FN inhibitors potentiate effects of PTX by interfering with cell attachment to the FN-rich ECM, without affecting cell viability or cell cycle progression.

Discussion

Our results provide data on newly developed SMIs targeting the interaction between TG2 and FN. We had previously reported TG53 as a lead inhibitor selected from high throughput screening of the ChemDiv library using an Alpha-Lisa assay (13). Here we characterize a series of analogues developed by using rational medicinal chemistry design and show that the analogues blocked TG2-FN interaction in vitro and OC cell adhesion to FN and to reconstituted peritoneal matrices in vitro and in vivo. Furthermore, the lead analogue, MT-4 prevented formation of stable focal contacts and mature focal adhesions to FN. Such compounds are predicted to block metastasis and to increase anoikis of unattached cells in the peritoneal cavity. These data have several implications.

First, we provide information on a new class of SMIs which block integrin-dependent adhesion and “outside-in signaling”. We show that chemical optimization of the “hit” diaminopyrimidine compound TG-53 was possible. The strategy aimed to improve potency and solubility of the hit compound by modifying its amide group. The derivative compounds demonstrated selective binding to TG2 and improved solubility (TG53<5 μM vs. MT-4; 91.5 μM and MT-6; 6.4 μM). While the proposed inhibitors demonstrate activity in vitro at low M concentrations, in cellular and in vivo models, higher concentrations were required to attain the desired effects, suggesting that future chemical optimization of these compounds will be needed to improve their potency. Noteworthy, biophysical measurements using BLI and ITC revealed that MT-4 bound directly to TG2 and prevented its interaction with the FN 45 kDa fragment. This suggested that in the presence of the inhibitor, TG2 would be prevented from bridging integrin β1 and FN in the ECM, hindering cell adhesion to the matrix.

Confocal microscopy supported this assumption, as TG2 colocalization with integrin β1 and FN in the adhesion complexes was decreased by the SMIs. In the absence of a strong interaction with the matrix, integrin β1 was displaced from the cell cortex, as shown by TIRF microscopy. Consequently, OC cells formed loose, unstable focal adhesions, with cell displaying curly ruffles at the plasma membrane and loss of cell polarity due to decreased lamellipodia in the presence of the inhibitors. This translated in a dose-dependent decrease of OC cells attachment to FN upon pre-treatment with the SMIs, as measured by solid phase assays. As TG2 was previously shown to induce integrin clustering independent from integrin-ligand interaction (21) and we observed partial loss of integrins colocalization in the presence of the SMIs, we inferred that by disrupting the interaction of TG2 with FN, integrin translocation from adhesion contacts was prevented and formation of an FN matrix could be inhibited (27). By preventing the accumulation of fibrillar FN in the tumor ECM, these TG2-FN SMIs could have a long-term effect on tumor cells' attachment to peritoneal sites. Additionally, these inhibitors will permit an in-depth evaluation of molecular events linked to integrin-dependent cell adhesion in contexts other than cancer.

FN is one of the most abundant ECM proteins in omentum and peritoneum (28). Adhesion of OC cells to FN via α5β1 integrin activates “outside-in signaling” by inducing phosphorylation of FAK, either directly (29) or through c-Met (30). This leads to activation of both survival (AKT) and mitogenic (MAPK) pathways (31), supporting cell proliferation and tumor growth (32). The 01 integrin-FN complex is strengthened by interactions with TG2, a protein we discovered to be overexpressed in OC (33). Here we show that inhibition of the TG2/FN complex by the new SMIs also delays and attenuates activation of FAK in response to cell adhesion. We had previously demonstrated the significance of TG2 to peritoneal dissemination through a β1 integrin dependent mechanism (3), as well as the significance of the TG2/FN/integrin complex to the survival of OC stem cells (5). Our current results using SMIs are consistent with our previous observations showing that targeting this complex, by using a function blocking antibody against the FN-binding domain of TG2 decreased spheroid proliferation and tumor initiation capacity (34). Together, these data support that the TG2/FN complex is an important cancer target.

Second, we provide here the first in vivo evidence on the potential of TG2-FN inhibitors to interfere with peritoneal metastasis. SKOV3 cells that were pretreated with either MT-4 or MT-6 before injection in the peritoneal cavity of immunodeficient mice, attached less efficiently to the peritoneal walls, as illustrated by the increased recovery of these cells 2 hours after inoculation. These findings were also corroborated by the solid phase assays performed by using LP9 cells, as a model of the peritoneal mesothelial lining, which showed that fewer cells attached to the mesothelial monolayer in the presence of the SMIs.

Similar cell-adhesion blocking strategies under development include the use of function-inhibiting antibodies against integrin receptors or against the hyaluronan receptor, CD44. These agents were shown to prevent the attachment of OC cells to the mesothelial monolayer (35-37). As a501 integrin is expressed on both OC tumor cells as well as on endothelial cells (38), targeting this heterodimer was expected to interfere with tumor growth and dissemination in solid tumors, including in OC (30). Although promising in preclinical models, anti-integrin antibodies have not yet demonstrated clinical benefit in patients with advanced stage solid tumors (39). This could be due to their initial testing in late stages of the disease, after metastases had been established. Future refinement of this strategy targeting adhesion to the peritoneum warrants further investigation, in settings preceding wide-spread tumor dissemination or in combination with standard cytotoxic therapy.

Third, we show that priming of OC cells with TG2/FN inhibitors, sensitized cells to paclitaxel. A potential mechanism underlying this interaction may be related to inhibition of “outside-in signaling”, which provides survival signals. We and others have previously shown that TG2 knock down increased response to chemotherapy in breast, ovarian and pancreatic cancer (12, 40-42), but this is the first demonstration that inhibition of the interaction with FN sensitizes cancer cells to chemotherapy. This concept deserves further exploration along with future testing of potential synergistic combinations with other cytotoxic or biological anti-cancer agents. In all, our results support further development of TG2/FN blockade as a new strategy to inhibit peritoneal metastasis and/or to sensitize cancer cells to standard cytotoxic therapy. Further medicinal chemistry-based optimization of this class of compounds is necessary to improve their properties and bioavailability in vivo.

### REFERENCES

1. Naora H, Montell D J. Ovarian cancer metastasis: integrating insights from disparate model organisms. Nat Rev Cancer. 2005; 5(5):355-66.
2. Akimov S S, Krylov D, Fleischman LF, Belkin AM. Tissue transglutaminase is an integrin-binding adhesion coreceptor for fibronectin. J Cell Biol. 2000; 148(4):825-38.
3. Satpathy M, Cao L, Pincheira R, Emerson R, Bigsby R, Nakshatri H, et al. Enhanced peritoneal ovarian tumor dissemination by tissue transglutaminase. Cancer Res. 2007; 67(15):7194-202.
4. Shao M, Cao L, Shen C, Satpathy M, Chelladurai B, Bigsby R M, et al. Epithelial-to-mesenchymal transition and ovarian tumor progression induced by tissue transglutaminase. Cancer Res. 2009; 69(24):9192-201.
5. Condello S, Sima L E, Ivan C, Cardenas H, Schiltz G E, Mishra R K, et al. Tissue transglutaminase regulates interactions between ovarian cancer stem cells and the tumor niche. Cancer Res. 2018.
6. Steeg P S. Tumor metastasis: mechanistic insights and clinical challenges. Nat Med. 2006; 12(8):895-904.
7. Buczek-Thomas J A, Chen N, Hasan T. Integrin-mediated adhesion and signalling in ovarian cancer cells. Cell Signal. 1998; 10(1):55-63.
8. Cao L, Shao M, Schilder J, Guise T, Mohammad K S, Matei D. Tissue transglutaminase links TGF-beta, epithelial to mesenchymal transition and a stem cell phenotype in ovarian cancer. Oncogene. 2012; 31(20):2521-34.
9. Satpathy M, Shao M, Emerson R, Donner D B, Matei D. Tissue transglutaminase regulates matrix metalloproteinase-2 in ovarian cancer by modulating cAMP-response element-binding protein activity. The Journal of biological chemistry. 2009; 284(23):15390-9.
10. Condello S, Cao L, Matei D. Tissue transglutaminase regulates beta-catenin signaling through a c-Src-dependent mechanism. FASEB journal: official publication of the Federation of American Societies for Experimental Biology. 2013.
11. Yakubov B, Chelladurai B, Schmitt J, Emerson R, Turchi J J, Matei D. Extracellular tissue transglutaminase activates noncanonical NF-kappaB signaling and promotes metastasis in ovarian cancer. Neoplasia. 2013; 15(6):609-19.
12. Cao L, Petrusca D N, Satpathy M, Nakshatri H, Petrache I, Matei D. Tissue transglutaminase protects epithelial ovarian cancer cells from cisplatin-induced apoptosis by promoting cell survival signaling. Carcinogenesis. 2008; 29(10):1893-900.
13. Yakubov B, Chen L, Belkin A M, Zhang S, Chelladurai B, Zhang Z Y, et al. Small molecule inhibitors target the tissue transglutaminase and fibronectin interaction. PLoS One. 2014; 9(2):e89285.
14. Shah N B, Duncan T M. Bio-layer interferometry for measuring kinetics of protein-protein interactions and allosteric ligand effects. J Vis Exp. 2014(84):e51383.
15. Wartchow C A, Podlaski F, Li S, Rowan K, Zhang X, Mark D, et al. Biosensor-based small molecule fragment screening with biolayer interferometry. J Comput Aided Mol Des. 2011; 25(7):669-76.
16. Condello S, Cao L, Matei D. Tissue transglutaminase regulates beta-catenin signaling through a c-Src-dependent mechanism. FASEB J. 2013; 27(8):3100-12.
17. Cardoso I, Osterlund E C, Stamnaes J, Iversen R, Andersen J T, Jorgensen T J, et al. Dissecting the interaction between transglutaminase 2 and fibronectin. Amino Acids. 2017; 49(3):489-500.
18. Folk J E, Cole P W. Mechanism of action of guinea pig liver transglutaminase. I. Purification and properties of the enzyme: identification of a functional cysteine essential for activity. J Biol Chem. 1966; 241(23):5518-25.
19. Hang J, Zemskov E A, Lorand L, Belkin A M. Identification of a novel recognition sequence for fibronectin within the NH2-terminal beta-sandwich domain of tissue transglutaminase. J Biol Chem. 2005; 280(25):23675-83.
20. Partridge M A, Marcantonio E E. Initiation of attachment and generation of mature focal adhesions by integrin-containing filopodia in cell spreading. Mol Biol Cell. 2006; 17(10):4237-48.
21. Janiak A, Zemskov E A, Belkin A M. Cell surface transglutaminase promotes RhoA activation via integrin clustering and suppression of the Src-p190RhoGAP signaling pathway. Mol Biol Cell. 2006; 17(4):1606-19.
22. Poulter N S, Pitkeathly W T, Smith P J, Rappoport J Z. The physical basis of total internal reflection fluorescence (TIRF) microscopy and its cellular applications. Methods Mol Biol. 2015; 1251:1-23.
23. Worth D C, Parsons M. Advances in imaging cell-matrix adhesions. J Cell Sci. 2010; 123(Pt 21):3629-38.
24. Bast R C, Jr., Hennessy B, Mills G B. The biology of ovarian cancer: new opportunities for translation. Nat Rev Cancer. 2009; 9(6):415-28.
25. Kenny H A, Nieman K M, Mitra A K, Lengyel E. The first line of intra-abdominal metastatic attack: breaching the mesothelial cell layer. Cancer Discov. 2011; 1(2):100-2.
26. Kenny H A, Dogan S, Zillhardt M, A K M, Yamada S D, Krausz T, et al. Organotypic models of metastasis: A three-dimensional culture mimicking the human peritoneum and omentum for the study of the early steps of ovarian cancer metastasis. Cancer Treat Res. 2009; 149:335-51.
27. Clark K, Pankov R, Travis M A, Askari J A, Mould A P, Craig S E, et al. A specific alpha5beta1-integrin conformation promotes directional integrin translocation and fibronectin matrix formation. J Cell Sci. 2005; 118(Pt 2):291-300.
28. Kenny H A, Lengyel E. MMP-2 functions as an early response protein in ovarian cancer metastasis. Cell Cycle. 2009; 8(5):683-8.
29. Schlaepfer D D, Jones K C, Hunter T. Multiple Grb2-mediated integrin-stimulated signaling pathways to ERK2/mitogen-activated protein kinase: summation of both c-Src- and focal adhesion kinase-initiated tyrosine phosphorylation events. Mol Cell Biol. 1998; 18(5):2571-85.
30. Mitra A K, Sawada K, Tiwari P, Mui K, Gwin K, Lengyel E. Ligand-independent activation of c-Met by fibronectin and alpha(5)beta(1)-integrin regulates ovarian cancer invasion and metastasis. Oncogene. 2011; 30(13):1566-76.
31. Renshaw M W, Price L S, Schwartz M A. Focal adhesion kinase mediates the integrin signaling requirement for growth factor activation of MAP kinase. J Cell Biol. 1999; 147(3):611-8.
32. Ward K K, Tancioni I, Lawson C, Miller N L, Jean C, Chen X L, et al. Inhibition of focal adhesion kinase (FAK) activity prevents anchorage-independent ovarian carcinoma cell growth and tumor progression. Clin Exp Metastasis. 2013; 30(5):579-94.
33. Matei D, Graeber T G, Baldwin R L, Karlan B Y, Rao J, Chang D D. Gene expression in epithelial ovarian carcinoma. Oncogene. 2002; 21(41):6289-98.
34. Condello S, Sima L, Ivan C, Cardenas H, Schiltz G, Mishra R K, et al. Tissue Tranglutaminase Regulates Interactions between Ovarian Cancer Stem Cells and the Tumor Niche. Cancer Res. 2018; 78(11):2990-3001.
35. Strobel T, Cannistra S A. Beta1-integrins partly mediate binding of ovarian cancer cells to peritoneal mesothelium in vitro. Gynecol Oncol. 1999; 73(3):362-7.
36. Cannistra S A, Kansas G S, Niloff J, DeFranzo B, Kim Y, Ottensmeier C. Binding of ovarian cancer cells to peritoneal mesothelium in vitro is partly mediated by CD44H. Cancer Res. 1993; 53(16):3830-8.
37. Strobel T, Swanson L, Cannistra S A. In vivo inhibition of CD44 limits intra-abdominal spread of a human ovarian cancer xenograft in nude mice: a novel role for CD44 in the process of peritoneal implantation. Cancer Res. 1997; 57(7):1228-32.
38. Slack-Davis J K, Atkins K A, Harrer C, Hershey E D, Conaway M. Vascular cell adhesion molecule-1 is a regulator of ovarian cancer peritoneal metastasis. Cancer Res. 2009; 69(4):1469-76.
39. Raab-Westphal S, Marshall J F, Goodman S L. Integrins as Therapeutic Targets: Successes and Cancers. Cancers (Basel). 2017; 9(9).
40. Verma A, Guha S, Diagaradjane P, Kunnumakkara A B, Sanguino A M, Lopez-Berestein G, et al. Therapeutic significance of elevated tissue transglutaminase expression in pancreatic cancer. Clin Cancer Res. 2008; 14(8):2476-83.
41. Verma A, Guha S, Wang H, Fok J Y, Koul D, Abbruzzese J, et al. Tissue transglutaminase regulates focal adhesion kinase/AKT activation by modulating PTEN expression in pancreatic cancer cells. Clin Cancer Res. 2008; 14(7):1997-2005.
42. Verma A, Mehta K. Tissue transglutaminase-mediated chemoresistance in cancer cells. Drug Resist Updat. 2007; 10(4-5):144-51.

### Example 2—Grant Application

A. Specific Aims

Ovarian cancer (OC) is the leading cause of death among gynecological malignancies¹ and metastasis to abdominal organs is the most common cause of OC mortality. Importantly, OC metastasizes by direct exfoliation of cancer cells from the primary tumor and intraperitoneal (ip) dissemination, without the requirement for vascular invasion. Adhesion to the extracellular matrix (ECM) plays an important role in the initiation of peritoneal metastasis. We identified tissue transglutaminase (TG2), an enzyme involved in Ca⁺⁺-dependent protein post-translational modifications and which has a well defined binding site for fibronectin (FN), as being highly expressed in OC²

Work in our laboratory over the past decade demonstrated that TG2 critically regulates the process of OC metastasis²⁻⁴. We showed that when injected ip or under the ovarian bursa of nude mice, OC cells engineered to express decreased levels of TG2 disseminated less efficiently compared to control cells. Our mechanistic studies have pinned down the interaction between TG2 and fibronectin (FN) as being a critical player in this process. We demonstrated that the TG2/FN complex is implicated in OC metastasis via multiple mechanisms: a) adhesion to the ECM by strengthening of integrin-dependent cell-matrix adhesion², b) induction of epithelial to mesenchymal transition (EMT)^{5,6}, c) regulation of Wnt/β-catenin signaling⁷, through direct interaction with the Frizzled 7 (Fzd7) receptor, which in turn promotes cancer cell proliferation and persistence of a stem cell profile⁸, d) remodeling of the extracellular matrix⁴, and e) modulation of intracellular oncogenic signaling^{7,9}.

Based on these results, we now hypothesize that the TG2/FN interaction is targetable and that its disruption by small molecules will prevent cancer cell adhesion to the matrix and OC metastasis. Preliminary computational¹⁰ and high throughput screening (HTS) strategies led us to discover several promising hits¹¹ that potently blocked this protein-protein interaction (PPI). We posit that their characterization and biochemical optimization could lead to a new therapeutic strategy in OC. Furthermore, we hypothesize that use of small molecule inhibitors (SMI) will reveal novel mechanisms by which TG2/FN interaction affects OC metastasis and stemness.

To this end, we completed HTS by using a newly developed Alpha-Lisa assay that robustly measured the TG2/FN complex formation and identified a new class of diamino-pyrimidines as potent inhibitors for this PPI¹¹. We demonstrated that several of these small molecules potently blocked OC cell adhesion and migration and by using structure-activity relationship (SAR) strategies we optimized the structure of the lead compound and generated analogs with improved efficacy. Here we propose to further optimize the top inhibitors and to elucidate the mechanisms by which they block cell adhesion to the matrix by using our unique expertise and resources in the Keck Biophysics Center and the Center for Molecular Innovation and Drug Discovery at Northwestern University.

Specific Objectives: We Will Test the Hypothesis by Addressing Three Objectives:

Specific Aim 1: To develop potent and drug-like TG2/FN inhibitors through rational medicinal chemistry optimization. Structure-based lead optimization will generate compounds with improved potency and specificity. Analogues will be analyzed by measuring inhibition of TG2-FN complex formation, cell adhesion and migration, and oncogenic signaling. Promising compounds will also be screened for in vitro metabolism, solubility, and permeability to help define those with the most drug-like characteristics.

Specific Aim 2: To define the composition of the TG2/FN complex and the effects of its disruption in OC in-vitro and in-vivo models. We will use nanocapillary HPLC tandem mass spectrometry (MS/MS) to determine the presence of other protein components in the TG2/FN complex in OC cells and to measure differences in complex composition between cancer and normal epithelial cells. We will measure the effects of an antibody inhibitor for TG2/FN complex in OC xenograft models.

Specific Aim 3: To characterize in-vivo the bio-distribution, tolerability and anti-tumor effects of highest ranked TG2/FN inhibitors. Pharmacokinetic (PK) metrics and toxicology studies for top analogs will be measured in mice. Effects on tumor dissemination will be analyzed in ip and orthotopic OC models.

Expected outcomes: Successful characterization and optimization of the newly discovered small molecules in the proposed assays will provide the basis for future clinical applications. As TG2 is highly expressed in over 75% of ovarian tumors and in particular, in high-grade ovarian malignancies 4, we anticipate that the proposed anti-TG2/FN strategies will impact the majority of patients with OC. In the long term, this line of innovative research will develop new agents to prevent metastasis in a difficult to treat, fatal cancer.

B. Significance:

Scientific Premise: Our group was the first to report TG2 upregulation in OC 3 and to define its critical role in peritoneal metastasis². Based on our discoveries over the past decade^{2,4-7,9,12} mechanistically linking the TG2/FN/integrin β1 interaction to peritoneal metastasis and cancer stemness, we propose this protein complex as a new target in OC. We will employ biochemical and biological assays to further characterize this complex and to optimize newly discovered small molecules that disrupt it. Their development was based on a step-wise demonstration of the role of TG2 in OC stemming from our group's work^{2,4-7,9,12} and validated by others^{13,14} Successful optimization of the newly discovered compounds will provide the basis for future clinical applications.

OC peritoneal metastasis: The process of metastasis in solid tumors requires loss of cell-cell contact and breakdown of the basement membrane followed by tumor invasion into lymphatic or vascular channels¹⁵. By contrast, OC harbors a distinct pattern of metastasis, with hematogenous dissemination being uncommon and a typical pattern of tumor spread directly through the peritoneal cavity, leading to development of implants on mesentery and bowel⁶. OC cells are in direct contact with the overlying peritoneal surface and fluid and simple dislodgement from the primary tumor allows cells to float in the peritoneal space, where they adhere and form metastatic implants. In the peritoneal fluid, OC cells aggregate as spheroids, which provide protection from the stress imposed by the extracellular environment. Interactions between cancer cells and the peritoneal mesothelium activate “outside-in” signaling¹⁷ which stimulates cell proliferation, survival and tumor angiogenesis. Over the past decade, our group demonstrated sequentially that TG2 regulates EMT⁶, migration of cells from the primary site², formation of spheroids in the peritoneal fluid 5, and invasion into the peritoneum^{2,12}. These steps regulated by the protein through its interaction with FN contribute to establishment of peritoneal implants^{2,6} and render TG2 an attractive new cancer target.

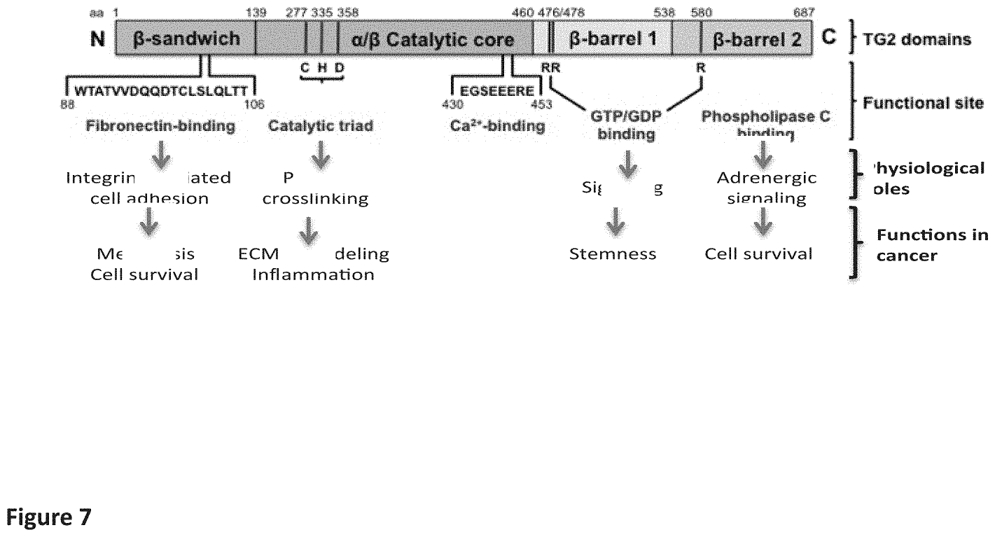

Tissue transglutaminase: structure-function dynamics. TG2 is a 76-kD protein, consisting of 686 amino acids and a member of the transglutaminase family, which includes TGT-7, Factor XIII and erythrocyte protein 4.2. They have similar catalytic activities and a four-domain tertiary structure, but restricted substrate specificity and distinct mechanisms of transcriptional regulation, leading to a tissue specific pattern of expression. TG2 has four domains: an N-terminus β-sandwich domain, which binds to FN, a catalytic triad C²⁷⁷H³³⁵D³⁵⁸, which carries out the acyl-transfer function, and two β-barrel domains^{18,19} (FIG. 7). A GTP/GDP-binding site is located between the catalytic and the first barrel domain, suggesting that TG2 functions as a GTP-ase. However, TG2 does not have a classical switch region, characteristic of G-proteins, and it is not clear whether and how GTP/GDP binding affects signaling mediated by TG2. TG2 also interacts with PLCγ through a region mapped at its C-terminus, supporting signaling from adrenergic receptors²⁰. The functions of the protein are modulated through large allosteric changes in the protein structure, which are tightly regulated in biological systems. For example, the crystal structure of guanine-nucleotide bound TG2 (PDB ID 1KV3) showed a compact conformation where the 2 β-barrel domains folded over the catalytic triad, obstructing the accessibility of Cys²⁷⁷, while the structure of the enzymatically active TG2 (PDB ID 2Q3Z) showed an open near-linear conformation exposing the catalytic core. In this “open” state, TG2 could not bind GTP/GDP, but was able to interact with substrates for transamidation. The conformation and functions of the protein are modulated by Ca²⁺ and GTP levels; high extracellular Ca²⁺ concentrations causing the protein to adopt an open structure and be enzymatically active, while high intracellular nucleotide levels force the protein to adopt a closed structure which blocks TG-ase activity²⁰. Thus, the physiological functions of TG2 are regulated by the cellular context and localization. At the plasma membrane, TG2 binds to the I₆II_{1,2}I₇₋₉ modules forming the 42 kDa gelatin-binding domain of FN (FN42) with high affinity (Kd ˜8-10 nM) and this complex provides a binding site for β1 and β3 integrins²¹⁻²³. TG2 also interacts with and stabilizes tubulin, microtubule binding proteins²⁴ and vimentin, contributing to stress fibers formation²⁵. As mutations in the catalytic core do not alter FN/integrin/TG2 complex formation, this role is independent of the transamidase function²². Within the cytosol, where Ca²⁺ concentrations are low, the majority of the protein is thought to assume a transamidation-inactive, closed conformation and it is believed that TG2 binds to GTP and to other protein partners, altering cellular signaling²⁰,2⁶. In the ECM, where Ca²⁺ concentrations are high and nucleotide concentrations are low, TG2 functions as a transamidase, facilitating Ca²⁺-dependent incorporation of amines into proteins and acyl-transfer between glutamine and lysine residues, leading to protein cross-linking and facilitating matrix remodeling²⁷. Multiple matrix proteins are known to be TG2 substrates, including FN²⁸, fibrin²⁹, osteopontin³⁰, laminin³¹, collagen³², and others. These functions suggest that TG2 plays important roles in cell adhesion, migration, and stromal assembly, key processes during cancer progression.

TG2 in cancer: Over the past decade several studies, to which our group has been a key contributor, have linked TG2 to cancer. Up-regulation of TG2 was reported in ovarian^{2,6} and pancreatic carcinoma³³, glioblastoma^{3,4}, lung³⁵ and breast cancer³⁶. TG2 overexpression was correlated with poor clinical outcome in pancreatic³⁷, ovarian³⁸ and lung cancer³⁹, suggesting that it functioned as a tumor promoter. Several oncogenic pathways (NF-κB, β-catenin, Rho, FAK, Akt, YAP) activated by TG2 have been implicated in cancer progression. In all, three themes have emerged. First, TG2 expression was linked to chemotherapy and radiation-resistance in cancer cells⁴⁰. This was attributed to activation of the NF-κB survival pathway^{41,42} and of “outside-in” signaling initiated through TG2-regulated cellular adhesion to the matrix³⁷. Second, TG2 has been linked to metastasis, our group having first reported that TG2 knock down in OC cells led to decreased peritoneal dissemination in ovarian orthotopic xenograft models^{2,6}. We reported that TG2 induced EMT^{2,6} and regulated cancer cell adhesion to the matrix to promote metastasis². Our observations were validated in breast and lung cancer models^{43,44}. Third, TG2 was found to be highly expressed in cancer stem cells (CSCs) first by our group 5 and subsequently, by others in breast⁴⁵, skin⁴⁶, and brain cancer models⁴⁷. While the evidence points to an oncogenic phenotype linked to TG2; there is still a lack of consensus regarding which functions of this protein promote cancer progression. Others and we have convincingly showed that interactions with integrins and FN at the plasma membrane are essential to activating outside in signaling (FAK, Akt, β-catenin, EGFR)^{13,48} and clearly implicated in cancer metastasis and stemness. However, in other contexts, the transamidase function was implicated in oncogenic activity: IκBα was shown to be crosslinked by TG2 leading to NF-κB activation⁴⁹; RhoA is transamidated by TG2 becoming constitutively active 5°. Studies using TG2 mutants (R^{580A} which is unable to bind GTP and C²⁷⁷ which is enzymatically inactive) have implicated the nucleotide binding function of TG2 in stemness^{46,51}. In summary, discoveries to which our group has contributed significantly during the past decade^{2,4-6,9-12,41,48,52-54} point to a pro-tumorigenic function of TG2 and the focus of this application is to target the TG2/FN/integrin complex.

C. Innovation: Several elements contribute to the highly innovative nature of this application. Conceptual innovation: The target (TG2/FN interaction) is novel and our group first characterized its roles in cancer metastasis and stemness^{5,7}. While past drug discovery efforts have focused on TG2's enzymatic activity⁵⁵, our strategy targets a previously un-tapped function of TG2, which we have clearly implicated in the process of metastasis and cancer stemness (see published work⁵⁷). The research approach is innovative, taking advantage of state of the art HTS technology and medicinal chemistry expertise available at our institution, and proposes a new class of inhibitors that will be studied in a disease of unmet medical need. Technical advancement: The proposed strategies rely on state of the art medicinal chemistry and novel proteomics strategies employing highly skilled collaborators and facilities. The TG2/FN inhibitors we are developing are new compounds that are being studied for the first time in cancer systems.

Team strength: A major asset of this proposal is the involvement of researchers who are leaders in the field of TG2 biology and biochemistry, gynecologic clinical practice, and medicinal chemistry (Matei-OC biology, therapeutics, Schiltz, medicinal chemistry, Wan and Goo, proteomics), and the resources of the Robert H Lurie Comprehensive Cancer Center (RHLCCC) and Northwestern University (NU), such as the patient-derived xenograft (PDX), Proteomics, and Developmental Therapeutics Cores, the Keck Biophysics Facility and the Center for Molecular Innovation and Drug Discovery. This project will foster a multidisciplinary and highly collaborative partnership to bridge a scientific gap and address fundamental biological questions related to metastasis in a deadly cancer.

D. Research Approach:

D1: Scientific premise and research strategy: The scientific premise for the proposed research is that the TG2/FN complex is critical to OC metastasis and stemness and can be targeted to prevent cancer progression. The goal of the proposed studies is to optimize and characterize a new class of small molecules, which inhibit the TG2-FN complex formation and were discovered through AlphaLISA based HTS applied to the ChemDiv library¹¹. The studies in Aim 1 will focus on the optimization of our series of TG2 inhibitors through rational medicinal chemistry to develop potent and drug-like compounds. Improved compounds will be studied in vitro to elucidate the mechanism by which they modulate the TG2-mediated oncogenic signaling, focusing on engagement of pathways we found to be regulated by the enzyme (0-catenin, c-Src). Biochemical and biological analyses of the SMIs will be followed by pre-clinical in vivo studies on toxicity, pharmacokinetics, and animal models of OC (Aim 3). The direct application will be reduction of OC metastasis. Additionally, the interaction between TG2-FN-integrins plays a role in resistance to chemotherapy^{43,49}, making possible testing future combinations with traditional cytotoxics.

D2. Our preliminary data and evidence in the literature support the scientific premise and the proposed approach: Key results are shown supporting that TG2 is an important cancer target and that the proposed experiments are feasible; details are outlined in published work 2,4-7,9,12

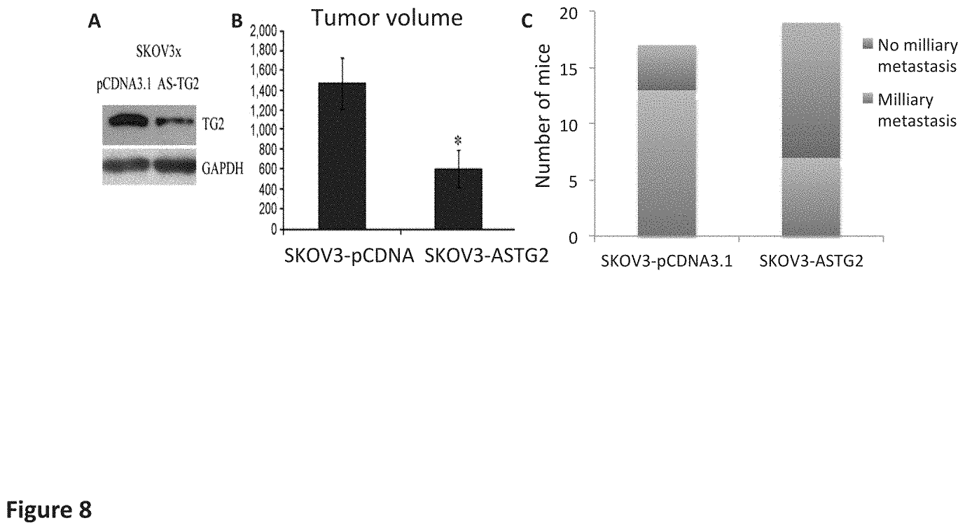

Evidence that TG2 critically regulates OC metastasis: We utilized both ip and orthotopic ovarian xenograft models derived from SKOV3 cells in which TG2 was stably knocked down by transfection with an antisense sequence (AS-TG2, FIG. 8A). Both models demonstrated that TG2 knock down blocked peritoneal metastasis^{2,6}. In the orthotopic model, the mean volume of primary tumors derived from SKOV3-AS-TG2 cells was smaller than controls (600±187 mm³ vs. 1468±257 mm³, p-value=0.01, FIG. 8B) and the number of metastatic implants was decreased in ASTG2 vs. control tumors bearing mice (FIG. 8C; p-value=0.02). These results strongly support TG2's role in promoting metastasis⁶. Key mechanistic elements involved are regulation of cell adhesion to the matrix and induction of EMT by TG^{2,4,6}. Additional evidence obtained by other groups supports the scientific premise that TG2 promotes metastasis in pancreatic and breast cancer models^{44,49}.

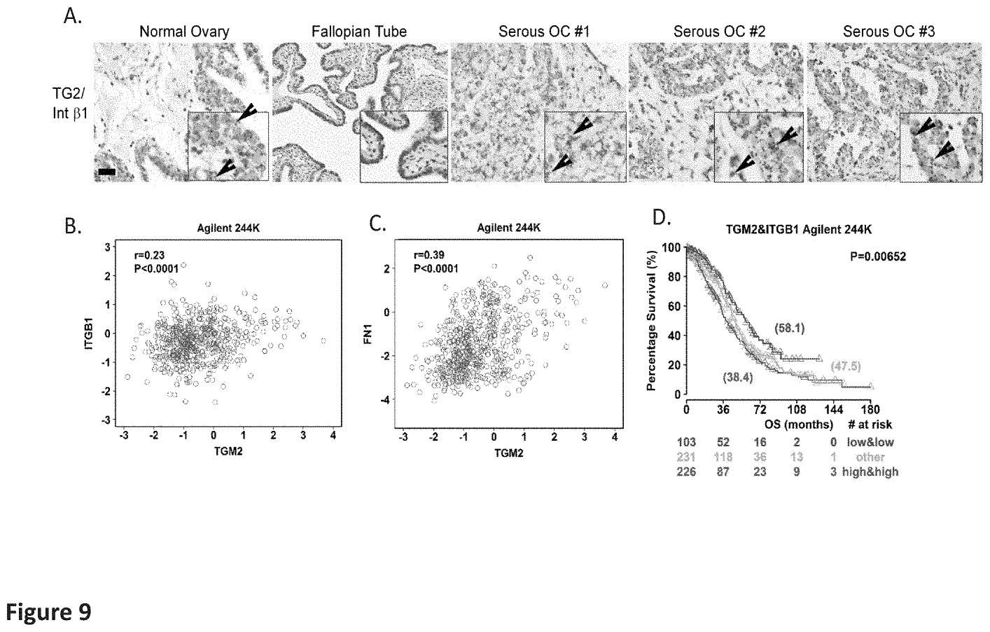

Evidence that the TG2/integrin β1 complex is detectable in OC. Although there remains a lack of consensus in the field regarding which specific function of the protein contributes to its oncogenic effects, our group has provided sequential and conclusive evidence that the complex formed by TG2 with FN and β-integrin plays an important role^{5,10,48}. However, the existence of a protein complex in vivo had not been demonstrated and its significance to clinical outcomes had not been proven. To determine whether this complex is detectable in human tumors, we used proximity ligation assay (PLA). Because TG2, FN and integrin β1 are part of the same complex, and staining for integrin β1 is more specific than for FN (diffuse ECM pattern), TG2 and integrin β1 co-localization was measured first in a set of 93 ovarian tumors. Complex formation was detectable in 85 of 93 ovarian malignant tumors, of which 43 displayed intense staining. The complex was not detected in normal surface ovarian or fallopian tube (FT) epithelium (n=7; FIG. 9A). We explored the TCGA database and showed that TG2 expression was strongly correlated with ITGB1 (R=0.23, p<0.0001, FIG. 9B) and with FN1 (R=0.39, p<0.0001, FIG. 9C). Furthermore, TG2 and ITGB1 were independent markers of survival and were included in a final multivariable analysis of overall survival. Patients with high TG2 and ITGB1 expression levels had an increased estimated risk of death when compared to those with low TG2 and ITGB1 expression levels (FIG. 9D). These data support the significance of TG2 at the interface with the ECM in human OC, impacting clinical outcomes 8.

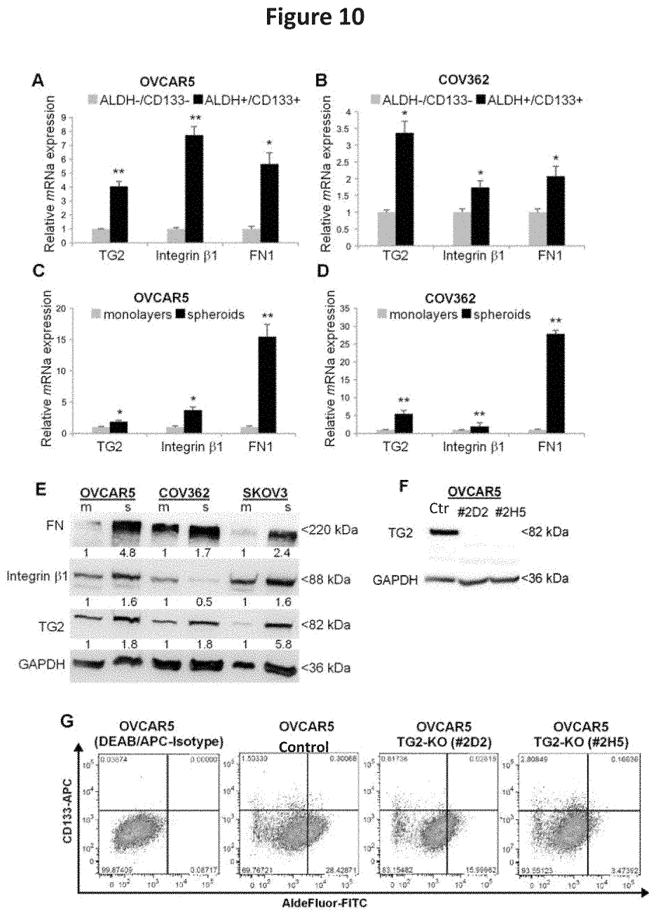

Evidence that the TG2/FN/β-integrin complex is enriched in OC stem cells: We recently demonstrated that TG2, FN1 and integrin β1 are highly expressed in ALDH⁺/CD133⁺ (CSCs) vs. ALDH⁻/CD133⁻ (non-CSCs) cells FACS sorted from OVCAR5 and COV362 (FIG. 10A-B). TG2, integrin β1 and FN expression levels were also increased in OC spheroids (rich in CSCs) vs. monolayers (low CSCs) across several OC cell lines and primary cells derived from malignant ascites at the mRNA (FIG. 10C-D) and protein levels (FIG. 10E). To further demonstrate the significance of TG2 to the CSC phenotype, the gene was edited using Crispr/Cas9 technology; generating two homozygous knock out (KO) clones in OVCAR5 cells (FIG. 10F). The ALDH⁺ and the ALDH⁺/CD133⁺ cell populations were significantly decreased in both clones, compared to control (FIG. 10G), supporting the critical role of TG2 in the maintenance of ovarian CSCs. TG2/integrin β1 co-localization by IF staining and quantified by Metamorph was also increased in CSCs-rich spheroids compared to monolayers (not shown, p<0.0001, see ref 7). Likewise, TG2 co-localized with cellular FN in both monolayer and spheroid cultures and significant enrichment of TG2-FN clusters was noted in CSC-rich 3D culture systems compared to monolayers (p<0.01, not shown). Collectively these data support the enrichment in TG2/FN/integrin β1 complexes in cancer cells growing as spheroids and suggest a key role of the complex supporting survival and proliferation of ovarian CSCs 8.

Discovery of small molecules that inhibit the TG2/FN interaction: Based on the demonstration that the TG2-FN complex plays a role in OC cell adhesion to the ECM², induction of EMT⁵, β-catenin and c-Src signaling⁷, which in turn regulate spheroid formation and the functions of CSCs, we set out to discover new small molecules that disrupt this complex. We pursued a step-wise strategy involving HTS and validation through biochemical and cell based assays, leading to the discovery of potent and specific inhibitors, of which TG53 showed the most potent bioactivity.

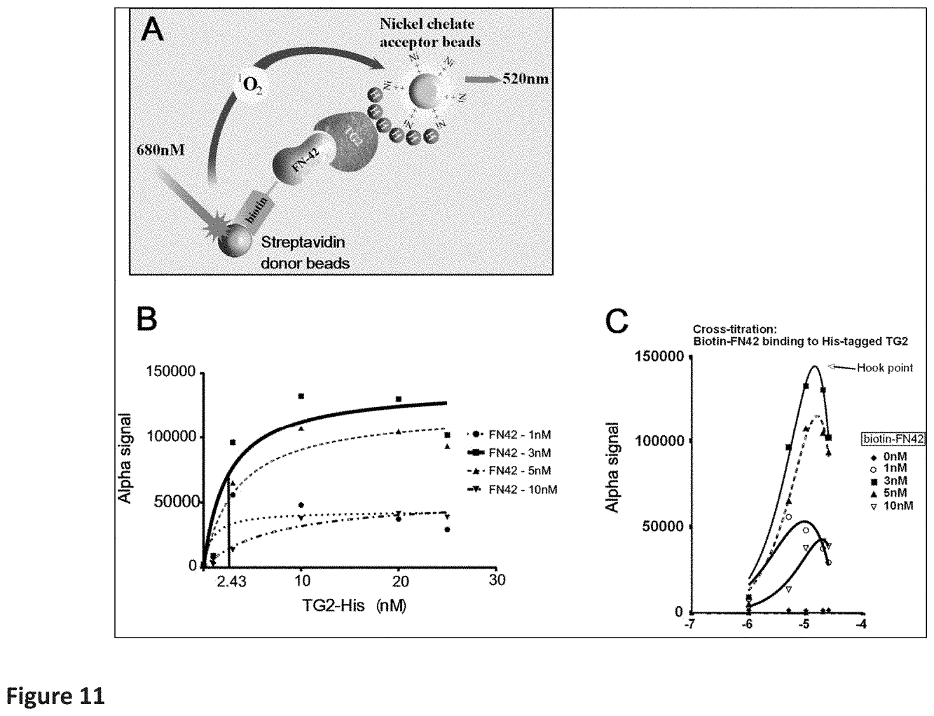

AlphaLISA assay measures TG2-FN interaction: We developed an AlphaLISA assay adaptable to HTS¹¹. The assay measured the interaction between His₆-TG2 bound to Ni-chelated acceptor beads and biotinylated FN42 bound to streptavidin coated donor beads (FIG. 11A). To determine optimal conditions, beads were incubated with serial concentrations of TG2 and the 42 kDa gelatin-binding domain of FN (FN42), and saturation curves determined the K_{d} for the interaction. Saturation was reached with 3 nM of FN42 and 10 nM of His₆-TG2, the K_{d} value being 2.43 nM (FIG. 11B). At “hook” point, when both beads were saturated, the maximum signal was detectable and excess protein above these levels inhibited the association between target molecules and beads causing decrease in signal. The hook point was reached at 10 nM of His₆-TG2 and 3 nM of FN42 (FIG. 11C). The Z′ value was calculated taking into account the means (μ) and the standard deviations (σ) of the positive and the negative controls. The average Alpha signal was 105757.4±9995.5 (mean±SD) and the estimated Z′ was 0.7, indicating robustness for HTS.

Discovery of TG2/FN inhibitors—HTS using the ChemDiv library: Next, the ChemDiv library containing 10,000 diverse compounds arrayed at 10 μM each was screened using the assay described¹¹. Primary hits (n=90) were selected as those compounds yielding ≥50% inhibition of control signal and were re-tested. Of those, 14 compounds were confirmed to have ≥70% inhibitory effect and were characterized further. The top 14 hits had a common core (N2(4-aminophenyl)pyrimidine-2,4-diamine) and belonged to two groups: one group of diamino-pyrimidines (TG37, TG40, TG49, TG50, TG53) and the other group of pyrolidinyl-pyrimidines (TG58, TG59, TG63, TG64, and TG65¹¹).

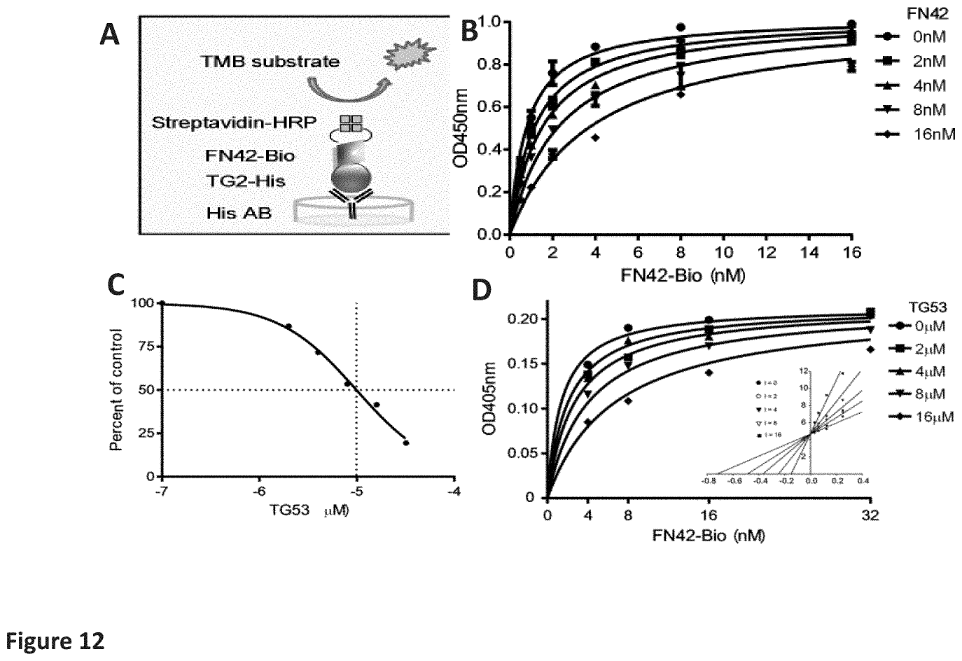

Confirmation using ELISA: An independent ELISA validated inhibition of TG2/FN complex formation by SMIs¹¹. The ELISA assay measured the binding between His₆-TG2 captured to the wells by anti-His antibodies and biotinylated FN42 detectable with streptavidin-HRP (FIG. 12A). Specificity of the assay was confirmed by using unlabeled FN42, which competitively blocked the interaction, abolishing the signal (FIG. 12B). Selected compounds (at concentrations between 5 and 10 μM) inhibited the ELISA signal by 29.7% to 69.5%¹¹. Of those, TG53 was the most active with 65% inhibition recorded at 5 μM. The inhibition constant Ki for TG53 was calculated as 4.15 μM from the effect of TG53 on FN42-Bio to TG2 (FIG. 12C). Lineweaver-Burk plot analysis of the binding data showed that TG53 competes for the same binding site in TG2 as FN42 (FIG. 12D). A structurally similar compound from the ChemDiv library which did not block the TG2-FN interaction in HTS (TG288) also did not block the ELISA signal (not shown, see¹¹).

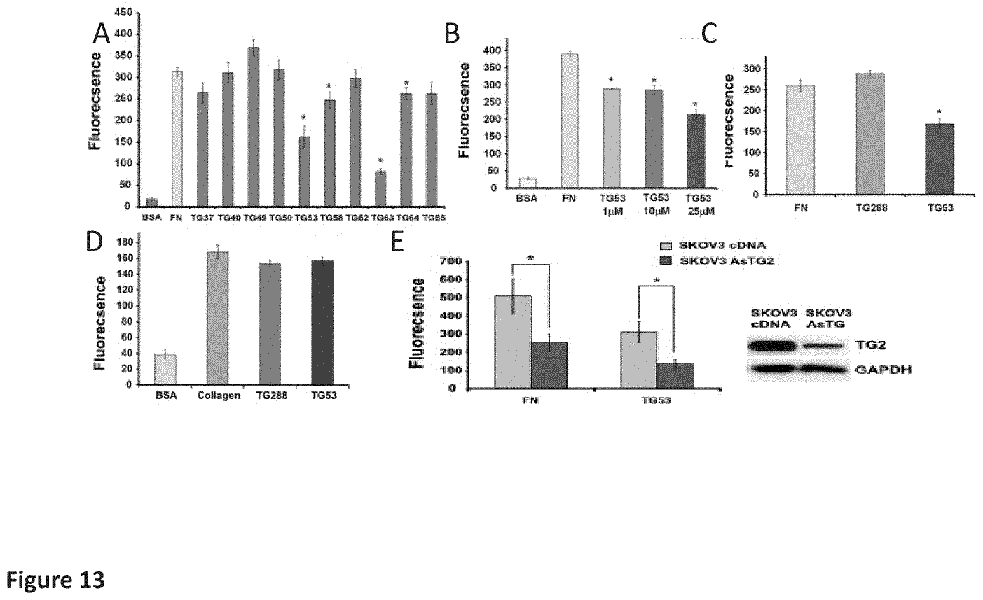

Selected SMIs blocked OC cell adhesion: We next studied the effects of top ranked SMIs on OC cell adhesion to FN. TG53, TG58, TG63 and TG64 inhibited cell adhesion to FN (FIG. 13A). TG53 demonstrated dose-dependent (FIG. 13B) and specific inhibition of cell adhesion to FN, compared to structurally similar, but inactive TG288 (FIG. 13C). Furthermore, TG53 did not inhibit adhesion to collagen, illustrating specificity to the target (FIG. 13D). Inhibition of cell adhesion to FN was more pronounced in TG2 expressing SKOV3pcDNA3.1 cells (control) compared to SKOV3-ASTG2 cells (FIG. 13E), further supporting TG2-specificity. Further characterization of TG53 demonstrated that the compound decreased wound closure, cell migration rate, and invasion through matrigel coated trans-well plates at concentrations of 10 μM, but did not inhibit TG2 enzymatic activity (not shown, see¹¹). TG53 was not cytotoxic at concentrations that caused TG2/FN complex disruption compared to other compounds, suggesting fewer off target effects. Based on those analyses, TG53 emerged as the lead TG2/FN inhibitor.

D3. Research Approach:

Specific Aim 1: To Develop Potent and Drug-Like TG2/FN Inhibitors Through Rational Medicinal Chemistry Optimization.

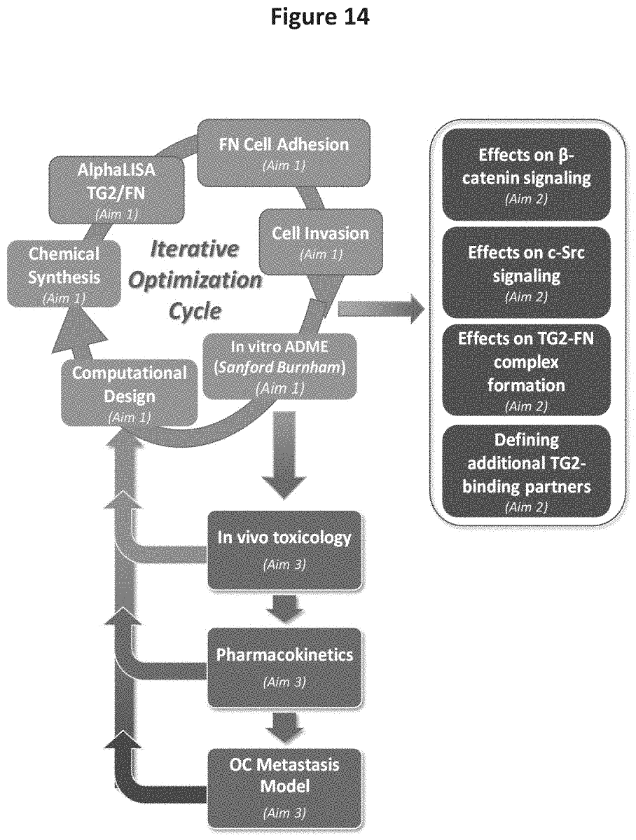

Rationale: The best hit TG53 exhibited good biochemical potency and highly efficacious cellular activity. However, the potency displayed by TG53 was still relatively modest, and not yet adequate for therapeutic development. Thus, improved and back-up compounds are needed to ensure successful preclinical evaluation of TG2/FN inhibitors. Given its promising potency, selectivity, and cellular efficacy, we hypothesize that TG53 serves as an excellent starting point upon which more potent and selective TG2/FN inhibitors can be developed. To this end, we will implement rational and iterative medicinal chemistry optimization of our lead series to generate TG2/FN inhibitors with improved potency, specificity, and pharmaceutical properties (FIG. 14). New compounds will be tested in a discovery funnel comprised of a series of increasingly relevant in vitro assays, including TG2/FN binding, FN cell adhesion and cell invasion, and in vitro absorption, distribution, metabolism, and excretion (ADME) studies. These efforts will identify the most promising TG2 inhibitors that will be used as chemical tools to further study the effects of inhibiting the TG2/FN interaction, and as therapeutic candidates for in vivo studies.

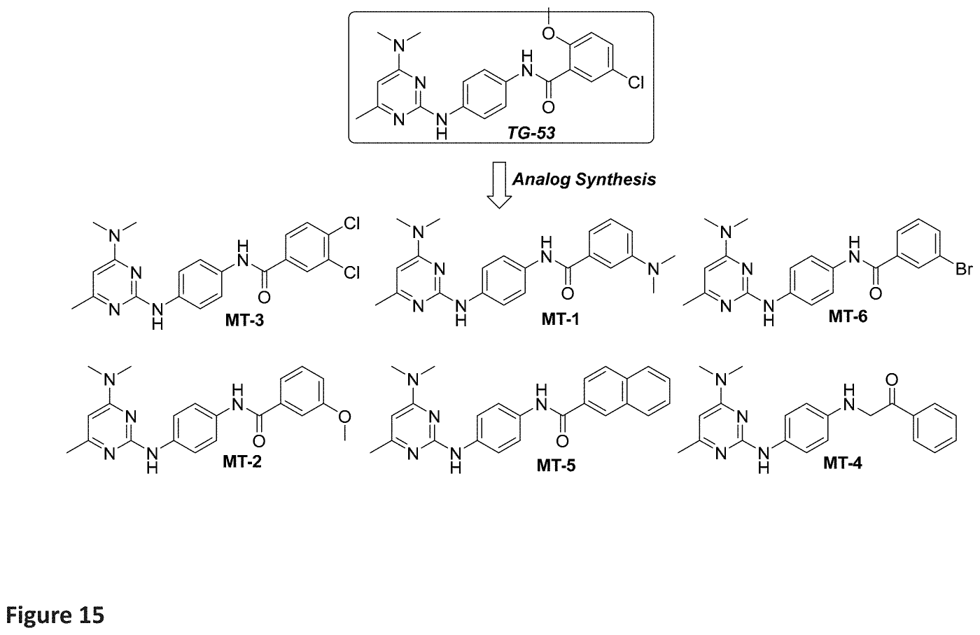

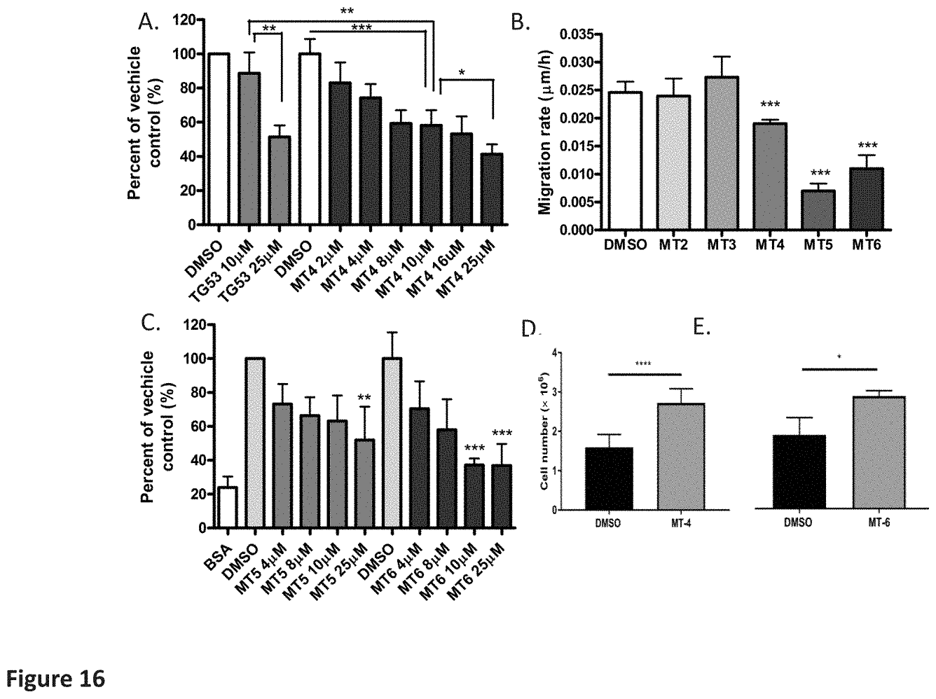

Preliminary SAR studies of TG53 involved the design and synthesis of 6 analogs (MT1-MT6, FIG. 15), which were characterized in vitro. All MT1-MT6 compounds inhibited TG2/FN formation by >50% as measured by ELISA (not shown). Subsequent biological assays showed the TG53 analogs blocked OC cell adhesion to FN (FIG. 16A) or to a layer of mesothelial cells mimicking the peritoneal surface (FIG. 16C) and inhibited cell migration in a transwell assay (FIG. 16B), consistent with their anticipated effects on the TG2/FN protein complex. We also developed a new assay to measure the effects of the inhibitors in vivo on cell adhesion to the peritoneal matrix. For this, GFP labeled OC cells were injected i.p. with or without SMIs (MT4 and MT6) in nude mice. Mice were euthanized 2 hours later and unattached GFP labeled cells were counted the peritoneal lavage. Our results show that both MT4 and MT6 prevented the adhesion of cancer cells to the peritoneum, resulting in an increased number of unattached cells (FIG. 16D-E), and demonstrating the compounds are active in vivo. The SMIs also altered cell signaling initiated by adhesion to the matrix (Phosphorylation of FAK and ILK, not shown). These findings confirmed that the new analogs block cell adhesion, which is critical during OC peritoneal metastasis, proving that TG53 is a reliable scaffold for new compounds with improved potency.

Research Design:

Lead optimization: The HTS we carried out identified a number of closely structurally-related analogs that inhibited TG2/FN interaction¹¹. In addition, the initial SAR studies demonstrated the ability to create structural analogs that modulate bioactivity gives us high confidence that this compound series is tractable for generating thorough SAR and driving the optimization of more potent and drug-like compounds.

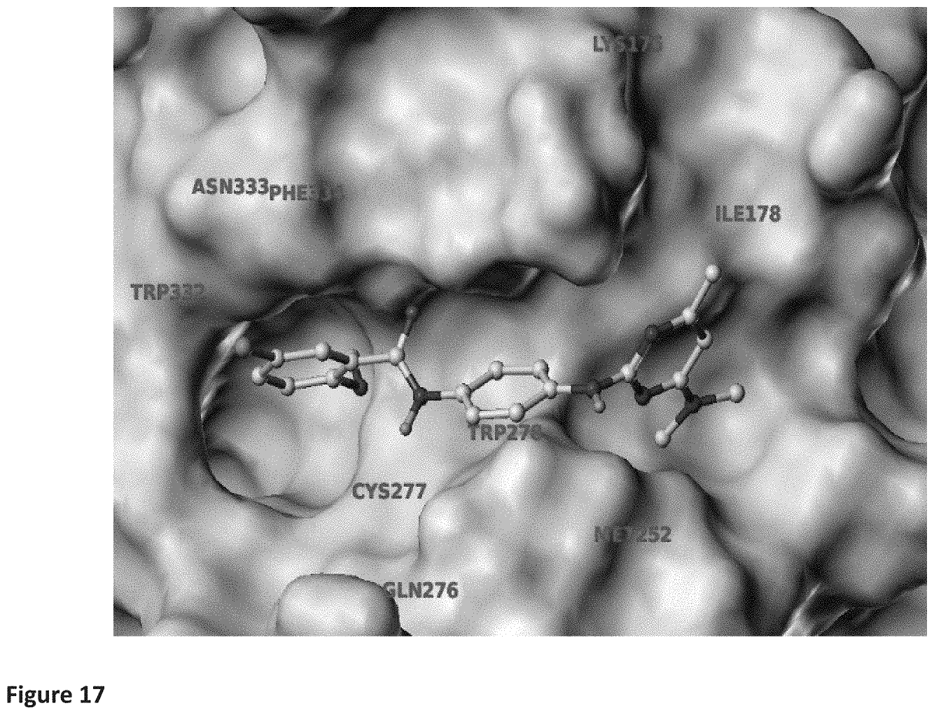

Building on our recent molecular modeling of TG2 protein interactions⁸, docking studies of our best inhibitor (TG53) shows a number of promising ways in which we could further improve binding potency (FIG. 17). Most obviously, there is a large hydrophobic pocket available behind the aryl ring. This open site could be exploited for increasing binding affinity by the addition of additional hydrophobic groups. As we design new compounds, we will first dock them into the TG2 binding site to help priorize which should be synthesized based on docked scores and binding energy calculations. In addition, we will calculate the predicted physiochemical and ADME properties of proposed compounds to support the synthesis of compounds expected to possess more pharmaceutical-like characteristics. These parameters include CLogP, molecular weight, solubility, permeability, and microsomal stability.

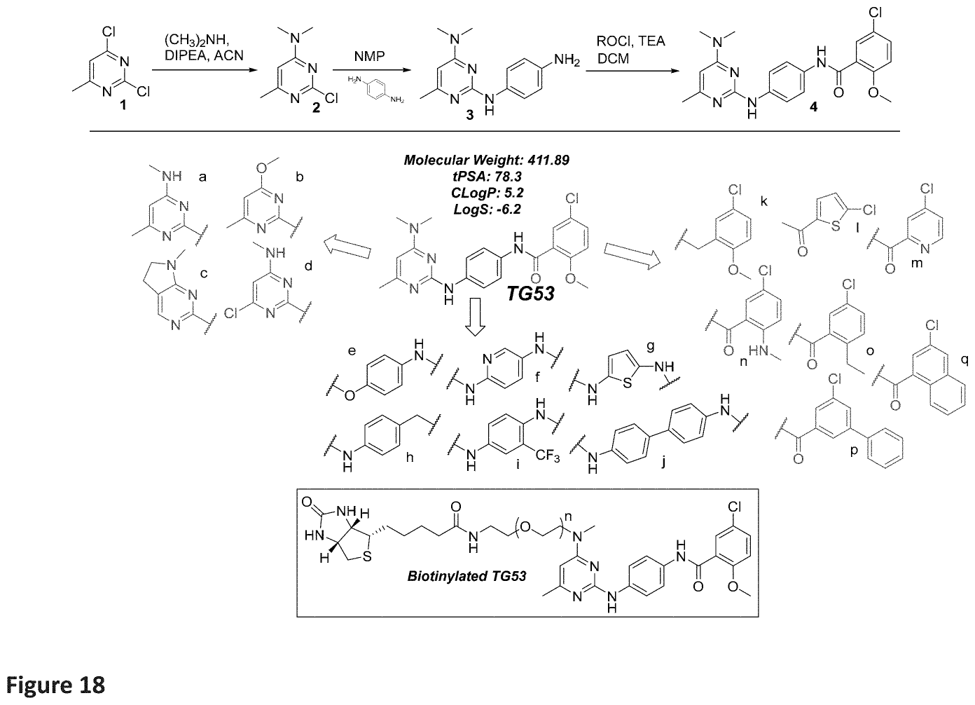

We expect to synthesize new compounds according to an efficient and straightforward 3-step route shown in FIG. 18. Based on our initial molecular modeling and general medicinal chemistry principles, a number of derivatives can be envisioned. A small set of representative examples is given in FIG. 18 as a way to explain our initial strategy. We will examine different electron-rich pyrimidine substituents such as the secondary amine (a) and the ether (b). These will probe the need for purely hydrogen-bond acceptor groups and explore other types of hydrogen-bond acceptors. Will also cyclize the aminoalkyl group to increase rigidity (c). The methyl group will also be replaced by a large number of substituents, including larger alkyl groups, hydrogen, aryl groups, and electron-withdrawing groups (d). On the center ring, our goal will be to increase solubility and eliminate the 1,4-diamino moiety which might be prone to oxidation. We will replace one or both of the nitrogens with other groups, such as ethers (e) and methylene (h). We will move the amines further apart by introducing a biaryl group (j). Other rings will also be explored, such as pyridines (which will also increase solubility, f), and classic benzene isosters such as thiophene (g). Finally, introduction of electron-withdrawing groups such as trifluoromethyl (i) may reduce the risk of oxidation. The right-hand side will be widely explored. A small set of examples include changing the linker type to sulfonamide, reversing the amide, and changing the amide to a secondary amine (k). We will also study different isosteres such as the thiophene (l) and the pyridine (which would also examine other hydrogen-bond acceptors, (m)). Also, the modeling (FIG. 17) suggests a large hydrophobic pocket extending off of the left-hand side and new analogs could also include larger alkyl groups (o) as well as biaryls (p) and naphthyls (q) to exploit this hydrophobic pocket. Finally, modeling (FIG. 17) suggests substituents on the pyrimidine ring are solvent-exposed. We will exploit this observation to synthesize biotinylated analogs of TG53 (FIG. 18). We may have to explore different linker types (e.g. PEG, alkyl) and linker lengths to identify one that displays similar activity in our AlphaLisa and other assays (see below) as the parent TG53. The potent biotinylated-TG53 will be used in studies described in Aim 2 for pulling down TG2 and its associated proteins. While only biotin-TG53 is shown in FIG. 18, we expect to prepare biotinyalted derivatives of other more potent analogs we create. All compounds will be purified by >95% purity, typically using preparative reverse-phase chromatography and fully characterized by ¹H-NMR, ¹³C-NMR, and MS.

In Vitro Screening of New Compounds

AlphaLISA TG2/FN assay: All compounds that are synthesized will be initially tested in our AlphaLISA assay, which was previously used for HTS¹¹ (FIG. 11). Compounds will be tested over at least a 3-4 log order dose range in triplicate to determine IC₅₀ of inhibition of the TG2/FN interaction. Compounds that possess potency in this assay better than our previous best compounds, or those that have IC₅₀<1 μM and superior drug-like properties, will be advanced to cell based assays. Potent compounds will be validated with ELISA.

FN Cell Adhesion: We have previously reported that the TG2-FN interaction is essential for regulating OC cell adhesion to the ECM. Compounds with improved potency in the AlphaLISA assay will then be studied for their effects on cell adhesion to FN by using SKOV3 and OVCAR5 cells, which endogenously express TG2. Dose response experiments in plates coated with FN will be performed. We will also use this assay to provide a measure of selectivity by testing adhesion to other substrates (e.g. collagen-1). To verify specificity, we will measure effects of SMIs on cells in which TG2 was knocked down (siRNA or Crispr/Cas9; cells are available; FIGS. 3 and 4). Our goal will be to advance compounds that inhibit cell adhesion to FN by >50% at <1 μM and do not show appreciable effects on cell adhesion to collagen-1. Lastly, effects of inhibitors on adhesion to a layer of mesothelial cells or to the peritoneal surface in vivo, will be measured with our newly developed assays for selected SMIs (FIG. 10).

Cell Invasion. Finally, compounds that possess good potency in inhibiting the TG2/FN interaction and in reducing cell adhesion to FN will be tested in assays measuring cell motility and invasiveness (wound healing and the trans-well invasion assays). Cell migration and invasion of SKOV3 and OVCAR5 cells incubated with a dose range of selected compounds will be measured. Our goal is to identify compounds that block cell migration and invasion rate by >50% at 10 μM. Compounds that meet these criteria will be advanced into in vitro ADME studies (below) as well as be available to use as tool compounds in Aim 2 to study the effects of potent TG2/FN inhibitors on OC.

Integration of in vitro ADME and selectivity profiling: Lead compounds with desirable potency and selectivity in our in vitro assays will be evaluated in standard ADME assays to assess solubility, metabolic potential, and PK. These studies will be performed under the supervision of Dr. Layton Smith Directory of the Sanford Burnham Prebys (SBP) Exploratory Pharmacology Core with whom we have worked on other drug discovery projects (see letter of support). The goals of these experiments are to 1) guide chemistry optimization to develop lead compounds with favorable drug-like properties, and 2) help prioritize molecules for subsequent in vivo studies. Solubility: Compounds will be evaluated for their kinetic and thermodynamic solubility at pH=7.4 (and possibly using SGF or SIF) using standard LC/MS-based methods⁵⁶ (Goal is solubility >5 μM). Metabolic Stability: Promising lead compounds will be tested for in vitro microsomal, S9, and plasma stability to determine the percent remaining and estimates of half-life. Compounds will also be tested with and without NADPH to identify non-CYP-dependent metabolism. Our goal will be to advance compounds with t_{1/2}>1 hr. Additional testing (e.g. toxicity, hERG inhibition, protein binding, permeability, PAMPA and Caco-2)) will also be carried out by Dr. Smith at SBP. In particular, PAMPA and Caco-2 data will be helpful in addressing any issues that arise from compounds that have good AlphaLISA potency but weak cell potency. These data will be used to design new analogs that have improved ADME properties and overcome any limitations that are identified.

Anticipated results, potential limitations, and alternative strategies: Based on our expertise, we anticipate our strategy to be successful. We also have confidence because our initial SAR studies demonstrated that new analogs can be synthesized to produce compounds that retain/improve TG2/FN inhibition, indicating that this is a tractable inhibitor scaffold. Creating biotinylated analogs that are also potent TG2/FN inhibitors may require us to make derivatives where the biotin is attached at other sites on the inhibitor. We expect that new analogs will disrupt the TG2-FN-integrin complex and cause inactivation of the pathways engaged by this complex at sub-μM concentrations. Ultimately this may affect cancer cell survival in the tumor matrix. Depending on results, we plan on testing selected SMIs found to block oncogenic signaling in combination with chemotherapeutics (e.g. cisplatin, paclitaxel, doxorubicin). In the event that the proposed TG53 analogs will not be significantly more potent than the parent drug, we will employ alternative strategies for hit optimization. For instance, biochemical optimization might use virtual screening of TG53 to identify new TG2/FN inhibitors that may be more tractable. We have previously successfully used virtual screening and docking for this PPI 57, and have experience employing this strategy for other targets⁵⁸⁻⁶¹ as well.

Specific Aim 2: To Determine the Composition of the TG2/FN Complex and the Effects of its Disruption in OC In-Vitro and In-Vivo Models.

Rationale and Preliminary Data:

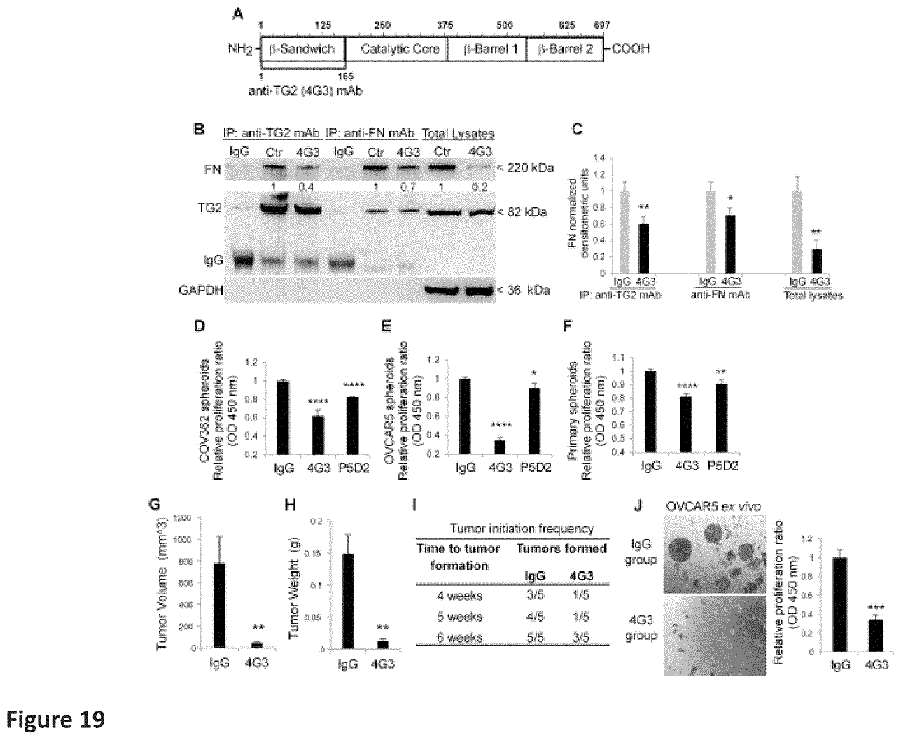

We demonstrated that TG2/FN/□1 integrin complexes are detectable in OC cells and tumors and are enriched in OCSCs. An inhibitory anti-TG2 monoclonal antibody (mAb) 4G3 raised against the N-terminal domain of TG2 (aa1-165), which binds FN (FIG. 19A) disrupted the TG2/FN interaction in ALDH⁺/CD133⁺ cells grown as spheres (FIG. 19B-C) and suppressed spheroids' proliferation compared to IgG isotype control in OVCAR5 (n=8, p<0.0001), COV362 cells (n=8, p<0.0001), and primary CSCs flow sorted from human OC ascites (n=4 specimens; FIG. 19D-F). By comparison, the function-blocking integrin D 1 antibody P5D2 blocked sphere proliferation less efficiently than 4G3 (FIG. 19D-F, p<0.05). Ex-vivo treatment with 4G3 blocked tumor-initiating capacity (TIC). Tumor volumes and weights derived from 4G3 vs. IgG pretreated ovarian CSCs (means±SEM) were significantly reduced (n=5 animals, p<0.01; FIG. 19G-H). Tumor initiation frequency was significantly delayed and inhibited by 4G3 vs. IgG control (FIG. 19I). Cells dissociated from 4G3 pre-treated tumors cultured ex vivo were not able to form spheroids compared to cells derived from control tumors (n=3, p<0.001, FIG. 19J) supporting that targeting the TG2-FN-integrin β1 complex disrupted OC stemness. Here we propose to test the effects of the 4G3 antibody in vivo in OC xenografts and PDX models.

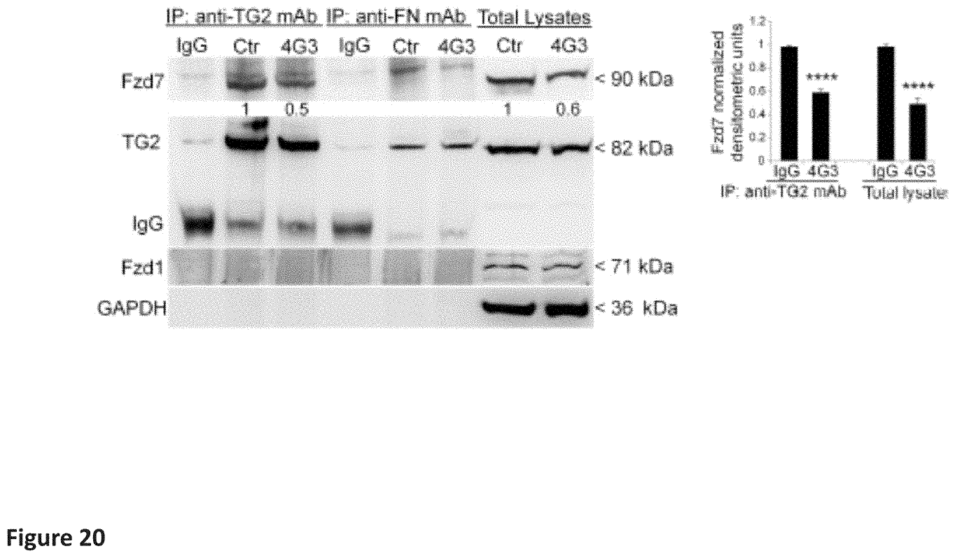

We next demonstrated that disruption of the TG2/FN complex blocked Wnt/□-catenin signaling (not shown, see⁸), leading us to test whether the Fzd receptors are part of this protein complex. Fzd7, but not Fzd1, was detected in endogenous protein lysates from CSCs pulled down with an anti-TG2 antibody (FIG. 20). The TG2-Fzd7 complex was disrupted by 4G3, suggesting that the area of interaction between TG2 and Fzd7 was located in the N-terminal domain of TG2 (amino acids 1-165), which is targeted by 4G3. In contrast, no interaction between FN and Fzd7 or between FN and Fzd1 was observed, as measured by co-IP with an anti-FN antibody. These data support that Fzd7 is anew substrate for TG2 and led us to hypothesize that other oncogenic proteins may be part of this complex. Here we propose to use mass spectrometry (MS) to more broadly determine the components of the TG2/FN complex in OC cells and to measure differences in complex composition between cancer and normal epithelial cells.

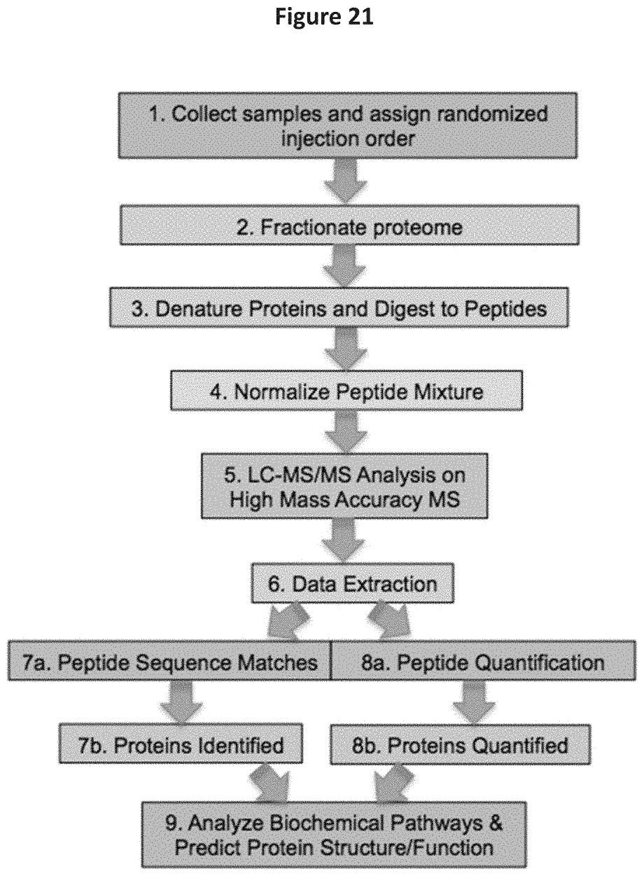

Design: Experiment 1: To test the hypothesis that TG2 forms distinct complexes at the interface with the ECM in malignant vs. normal epithelial cells and to define the composition of these complexes, we will use MS-based measurements and discovery analytical tools. We plan to compare protein complexes from fallopian tube (FT) epithelial cells (Lifetime Technology) vs. OC cells (cell lines-OVCAR5, OVCAR432, SKOV3; or primary OC cells). We will start with a comparison using whole cell lysates, followed by comparison of membrane fractions (enriched in complexes at the interface with the ECM) extracted by using high-speed centrifugation^{2,12}. Endogenous TG2 will be pulled down with a mAB (CUB7402), which efficiently immuno-precipitates the protein 8. Protein complexes will be purified by TCA/Aceton precipitation and digested. After desalting, digested peptides will be processed for MS acquisition. A shotgun proteomic process will be used based on nanocapillary HPLC tandem mass spectrometry (MS/MS; FIG. 21). Our current proven interactome profiling technology involves: 1) Immunoprecipitation of TG2/FN binding proteins; 2) optional fractionate proteome; 3) protein denaturation and proteolytic digestion of proteins to peptides; 4) normalization of peptide quantities for analysis; 5) HPLC-MS/MS analysis on an Orbitrap (OT) mass analyzer; 6) data extraction; 7a) peptide tandem mass spectral matches to sequence; 7b) protein identifications; 8a) extraction of changes peptide expression; 8b) calculation of protein relative quantities and 9) analysis of biochemical pathways indicated by quantitative changes in the proteomes. The approach uses data from a hybrid mass spectrometer, that combines two mass spectrometers each with a separate detector to allow simultaneous precursor ion scans in the high resolution high mass accuracy OT mass analyzer from which we derive quantitative data and tandem mass spectrometry in the ion trap mass analyzer from which we derive peptide sequence matches. Peptide identification. Acquired peptide tandem mass spectra are matched to sequence in databases and a best fit is generated by a sliding cross-correlation score. Typically, we use MASCOT (Matrix Science) and MaxQuant (Andromeda). Further algorithms, Peptide- and Protein-Prophet (Institute for Systems Biology, Seattle, Wash.) use statistical routines to assign probability scores to the peptide sequence best fit and Scaffold (Proteome Software) will be used for data visualization.

Calculating changes in protein expression: Differences in relative expression of proteins will be calculated using MaxQuant software tools, supported by Andromeda database search engine. Normalized total peptide signals within each run will be used for label-free quantitation, allowing protein ratios to be calculated. For label-free quantification, MaxQuant uses MS1 based ion intensity calculations from the maximum intensity over the retention time profile or peptide spectral counting that uses tandem mass spectrometry data (i.e., MS2) to more rapidly estimate by statistical means changes in relative abundance.

Biochemical pathway analysis will use MetaCore (Thomson Reuters) to reconstitute functional pathways. There are many options for this type analysis and we will use the best available. At the end of these experiments we anticipate identifying the TG2 complexes composition in cancer and normal FT cells. Individual proteins will be then validated by using IP, IF staining and other functional biological assays, as we have previously used^{8,48}. We anticipate that TG2 complexes will be enriched in surface proteins with oncogenic functions in cancer vs. normal cells.

Experiment #2: We will next test the effects of TG2/FN disruption on OC metastasis in vivo. For this we will use an ip approach and OVCAR5 xenograft model. 1×10⁶ cells will be injected ip in female 7-8 week-old nude mice. Treatment with 4G3 or IgG control will start at the time of the ip injection (1 mg/kg) and will be continued weekly. After 4 weeks, mice will be euthanized and the number of metastases, ascites and tumor volumes will be measured and compared, as in our published work^{2,5,6}. Similar experiments will use an ip PDX model in female NSG mice, which is available in our Developmental Therapeutics Core⁶². IHC of harvested tumors will measure TG2 and β-integrin expression, β-catenin nuclear localization, Ki-67 index, FAK and ILK phosphorylation. PLA will measure TG2/integrin complex formation in harvested tumors, as we described⁸, anticipated disruption of the complex in 4G3 treated tumors. Analysis: By using 10 animals per group we will achieve an 80% power to detect 0.7 SD difference between experimental groups, assuming that the SD in this experiment will be similar to our previous experiments^{2,6} Linear trends between experimental and control groups will be assessed through Wald linear-trend tests imbedded in Cox proportional-hazard regression model. The Wilcoxon two-sample non-parametric test will compare IHC H-scores (intensity X number of staining cells).

Anticipated results, pitfalls, and alternative approaches: Based on our preliminary data demonstrating that ex-vivo treatment of OCSCs with 4G3 suppresses sphere proliferation and TIC, we anticipate that the antibody will inhibit ip metastasis. We will use 4G3 at 1 mg/kg, which will lead to concentrations of ˜50 μg/ml in the peritoneal space, exceeding doses used in vitro. Based on these results, future or alternative experiments will include combinations with chemotherapy (platinum or paclitaxel) or with anti 01 integrin antibodies. The proteomics experiment will use the expertise of the NU Proteomics Core (Dr. Goo) and yield new information about novel TG2 substrates or TG2 partners that drive oncogenic signaling. We anticipate that there are distinct TG2 partners in cancer vs. normal cells. The lower levels of TG2 in normal FT epithelium, may limit the amount of TG2 complexes pulled down in normal cells, thus limiting the comparison. Even if we are not able to characterize the TG2 interactome in normal FT cells, identifying the protein partners in OC cells will provide sufficient new information. Given that TG2 is abundantly expressed in OC cells, we will start the experiments by pulling down endogenous TG, however, if insufficient endogenous complexes are purified, we are prepared to overexpress tagged His₆-Flag-TG2 and pull down protein complexes by using anti-His and anti-Flag antibodies.

Specific Aim 3: Characterize the Effects of TG2/FN Inhibitors in OC Animal Models.

Rationale: We anticipate that Aims 1 and 2 will identify analogues with improved potency and specificity and will better define the composition of the TG2/FN complex. Here, compounds with the greatest potency and in vitro ADME will be characterized in vivo. Pharmacokinetics (PK) and tolerability will be defined in mice not carrying tumors and anti-tumor activity will be measured in OC xenografts models.

Research Approach:

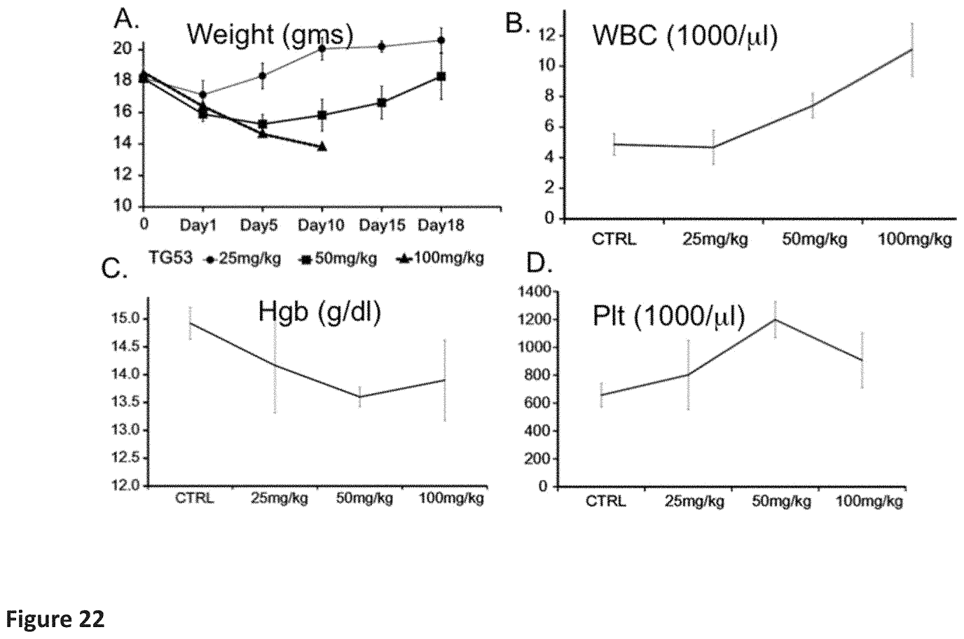

Experiment 1: Determination of lead compound acute toxicity profile. Compounds that possess desired bioactivity and in vitro metabolism/absorption (Aim 1) will be studied here. In these studies, we will establish a single dose ‘No-Observed-Adverse-Effect-Level (NOAEL)’ for our compounds in exploratory studies using only female FVB/N mice to minimize animal usage and to model our OC efficacy studies. We will perform a rising dose acute toxicology study starting at 25 mg/kg (3 mice per compound, repeated at up to 6 concentrations) and follow each animal for 48 hours following a single ip dose for evidence of acute toxicity. The dose will be escalated until a NOAEL is defined based on clinical observations. Terminal blood samples will be obtained and analyzed for complete blood count and chemistry. We anticipate testing 2 compounds per year (in Y2-Y3) and 4 compounds per year (in Y4-Y5) (total≈12 compounds). The NOAEL will be used to determine the highest dose for PK testing. Preliminary studies with TG53 showed that the drug was tolerated up to 50 mg/kg and did not induce hematological toxicity (FIG. 22) and therefore, we anticipate that the analogs will also be tolerated at similar doses. Toxicity will be determined by standard criteria: hunched posture, lack of grooming, failure to thrive, failure to eat and drink, loss of 15-20% body weight, loss of righting reflex. Subsequent studies will use daily dosing for 5 days testing 2-3 dose levels (cohorts of 3-5 mice) selected from the single dose acute toxicity study and considering PK data.

Experiment 2: Pharmacokinetic (PK) assessment. PK studies using the highest safe dose will be conducted on lead compounds that were functionally validated and for which a single dose NOAEL could be established (Aim 3A). These will be carried out by the Sanford Burnham Pharmacology Core (see attached LOS). While ip dosing may not be the preferred route of administration for an eventual cancer drug, these studies using ip dosing will give us data on many PK parameters, including bioavailability, which will be useful in prioritizing and triaging optimized compounds for testing using other routes, e.g., oral. PK parameters will be determined including Cmax (maximum plasma concentration), Tmax (time point of maximum concentration), VdSS (volume of distribution at steady-state), ClE (elimination clearance), AUC (area under the curve) t, (elimination half-life), and oral bioavailability (% F) will be obtained (with the inclusion of an i.v. dosed cohort). These data will determine if the plasma concentrations are sufficient to provide satisfactory data in animals in the efficacy studies and to determine appropriate doses and dosing schedules for promising compounds. Our goal will be to identify a compound that has a plasma AUC >5× its cell viability EC50, a t₁₋₂>2 hrs, and low-to-moderate clearance. Preliminary data for TG53 demonstrate a t/2 of 3.4 h for iv and 19.5 h for ip administration, supporting feasibility of the method and stability of the compound in vivo. Toxicology and pathology measures will be performed only after anti-tumor activity of lead SMIs will be determined, if compounds are found worthy of detailed study (Expt #3). This approach will maximize use of animals and resources. In brief, peripheral blood counts will be obtained; selected organs and tissues (lung, heart, liver, kidney, colon, bone marrow) will be harvested. Bone marrow cellularity and colony formation assay will be determined.

| | Route | | | | | | |
|---|---|---|---|---|---|---|---|
| Dosage | of | cₘₐₓ | tₘₐₓ | AUC_{0-∞} | t_{1/2} | Cl/F | Vdₛₛ/F |
| (mg/Kg) | Admin | (ng/mL) | (hours) | (ng*mL⁻¹*hr) | (hours) | (L/hr) | (L) |
| 17.46 | IV | 4045 | 0 | 2913 | 3.4 | 0.12 | 0.40 |
| 17.46 | i.p. | 1313 | 0.5 | 1212 | 19.5 | 0.29 | 0.40 |

Experiment 3: Determine anti-tumor activity and biological effects: The first experiments testing anti-cancer activity of the small molecules will use ip OC xenografts and ip administration to maximize the concentration of the compound in the ip space, where it will interfere with cell adhesion to the matrix. Female Balb c nu/nu mice, 7-8 weeks old will be injected ip with 2×10⁶ TG2 expressing OVCAR5 cells. Mice will be treated with selected compounds, at the dose and schedule established in Experiments #2-3 or vehicle (control). At necropsy, number and size of tumor implants in the peritoneal cavity will be recorded. If these first experiments are successful, then more complex models will be used to measure antitumor effects of selected analogs. Specifically, we will use the orthotopic OC model⁶ and initiate treatment 1 week after tumor implantation under the bursa using ip route. At necropsy, ascites, number and size of tumor implants will be recorded. Tumors will be analyzed by IHC for TG2, β-catenin, Ki-67 and CD31 expression. An “H score” will be calculated as the product between the intensity and the percent of staining cells. IgG will be used as negative control. Number of positive Ki67 cells and CD31⁺ tubular structures will be counted in 5 fields/specimen. If β-catenin signaling is disrupted, then we will quantify stem cell populations. Freshly harvested tumors will be minced to single cells and FACS will quantify ALDH1⁺/CD133⁺ cells^{5,63}, anticipating decreased percentage of CSCs in treated tumors vs. controls. Analysis: Effects of SMIs on tumor and metastasis will be based on the difference between the mean tumor volumes and number of peritoneal implants between groups. With 10 animals per group we will achieve 80% power to detect 0.7 SD difference between groups, assuming similar SD to our previous experiments^{2,6}. Linear trends between experimental and control groups will be assessed through Wald linear-trend tests imbedded in Cox regression model. Wilcoxon two-sample non-parametric will compare the IHC H scores between groups.

Experiment 4: Define bio-distribution: If antitumor activity is noted; then more detailed tissue distribution studies will be performed. Two cohorts of 6 tumor bearing mice each will be dosed at maximum tolerated dose (single dose iv (cohort 1) or ip (cohort 2)). Tissues (liver, kidney, lung, heart, peritoneum, tumors) will be homogenized and concentrations of SMIs will be determined. Timing of tissue harvest will take into account the t½: 3 mice/cohort will be harvested after one t½ and 3 mice/cohort after two t½. The analyses will determine whether compounds accumulate in specific tissues and penetrate tumors.

Anticipated results, limitations, and alternative strategies: The focus is to determine in vivo anti-tumor properties of top analogs. Toxicity studies will not be exhaustive, at this early stage of development, as to minimize costs and animal usage. The main goal is to determine a tolerable dose that will yield a sustained ip and plasma concentrations close to the IC50. Anti-tumor activity will be assessed as described. Luciferase-based bioluminescence imaging (BLI) using the Night Owl LB981 System could be used as an alternative to monitor disease progression. Other future studies may include combination regimens with chemotherapeutics (platinum or paclitaxel). We have extensive expertise with IHC staining^{4,7,64}. We expect that compounds will not alter the intensity of TG2 staining, but will cause decreased β-catenin nuclear localization, as disruption of TG2/FN/integrin complexes is expected to alter this pathway. If SMIs inhibit the transcriptional activity of β-catenin, then decreased proliferation indices in tumors from treated animals may be observed. There may be also other mechanisms blocked by these SMIs, such as disruption of angiogenesis, given that the TG2/β-integrin/FN complexes play a role in vascular remodeling⁶⁵ and interact with PDGFRβ in vascular smooth muscle and endothelial cells^{66,67} which will be examined.

Rigor, reproducibility and inclusion of sex in research: We will follow the following principles: (A) Rigor: A biostatistician was consulted for planning the experiments and estimating the required sample size, number of replicates and analysis plan. All cell lines and reagents used are authenticated. New compounds will be fully characterized by established analytical techniques. (B) Reproducibility: Multiple animal models, cell lines, methods of inhibition or activation of specific enzymes (pharmacological or genetic) will be used through the project. Staff blinded to type of drug treatment will verify IHC. At least 3 biological replicates will be used. (C) Gender as a variable: Only female animals will be used for tumor experiments; as OC is a gender-restricted malignancy; but mice of both genders will be used for PK and toxicity studies.

Future studies: At the project's completion, we expect to discover and characterize a first generation of TG2/FN inhibitors with improved in vitro and in vivo activity. This will allow us to proceed with preclinical development of at least 2-3 analogues. The first application is OC, however TG2-FN interaction is important in other malignancies, and the new SMIs may be tested in other tumor types. Combination regimens with cytotoxics will be planned.

### REFERENCES

1. Greenlee R T, Murray T, Bolden S, et al: Cancer statistics, 2000. CA Cancer J Clin 50:7-33, 2000.
2. Satpathy M, Cao L, Pincheira R, et al: Enhanced peritoneal ovarian tumor dissemination by tissue transglutaminase. Cancer Res 67:7194-202, 2007.
3. Matei D, Graeber T G, Baldwin R L, et al: Gene expression in epithelial ovarian carcinoma. Oncogene 21:6289-98, 2002.
4. Yakubov B, Chelladurai B, Schmitt J, et al: Extracellular tissue transglutaminase activates noncanonical NF-kappaB signaling and promotes metastasis in ovarian cancer. Neoplasia 15:609-19, 2013
5. Cao L, Shao M, Schilder J, et al: Tissue transglutaminase links TGF-beta, epithelial to mesenchymal transition and a stem cell phenotype in ovarian cancer. Oncogene 31:2521-34, 2012
6. Shao M, Cao L, Shen C, et al: Epithelial-to-mesenchymal transition and ovarian tumor progression induced by tissue transglutaminase. Cancer Res 69:9192-201, 2009
7. Condello S, Cao L, Matei D: Tissue transglutaminase regulates beta-catenin signaling through a c-Src-dependent mechanism. FASEB J, 2013
8. Condello S, Sima L E, Ivan C, et al: Tissue transglutaminase regulates interactions between ovarian cancer stem cells and the tumor niche. Cancer Res, 2018
9. Cao L, Petrusca D N, Satpathy M, et al: Tissue transglutaminase protects epithelial ovarian cancer cells from cisplatin-induced apoptosis by promoting cell survival signaling. Carcinogenesis 29:1893-900, 2008
10. Khanna M, Chelladurai B, Gavini A, et al: Targeting ovarian tumor cell adhesion mediated by tissue transglutaminase. Molecular cancer therapeutics 10:626-36, 2011
11. Yakubov B, Chen L, Belkin A M, et al: Small molecule inhibitors target the tissue transglutaminase and fibronectin interaction. PLoS One 9:e89285, 2014
12. Satpathy M, Shao M, Emerson R, et al: Tissue transglutaminase regulates matrix metalloproteinase-2 in ovarian cancer by modulating cAMP-response element-binding protein activity. The Journal of biological chemistry 284:15390-9, 2009
13. Verma A, Guha S, Wang H, et al: Tissue transglutaminase regulates focal adhesion kinase/AKT activation by modulating PTEN expression in pancreatic cancer cells. Clin Cancer Res 14:1997-2005, 2008
14. Verma A, Mehta K: Transglutaminase-mediated activation of nuclear transcription factor-kappaB in cancer cells: a new therapeutic opportunity. Curr Cancer Drug Targets 7:559-65, 2007
15. Steeg P S: Tumor metastasis: mechanistic insights and clinical challenges. Nat Med 12:895-904, 2006
16. Naora H, Montell D J: Ovarian cancer metastasis: integrating insights from disparate model organisms. Nat Rev Cancer 5:355-66, 2005
17. Buczek-Thomas J A, Chen N, Hasan T: Integrin-mediated adhesion and signalling in ovarian cancer cells. Cell Signal 10:55-63, 1998
18. Huang X, Lin T, Gu J, et al: Combined TRAIL and Bax gene therapy prolonged survival in mice with ovarian cancer xenograft. Gene Ther 9:1379-86, 2002
19. Stephens P, Grenard P, Aeschlimann P, et al: Crosslinking and G-protein functions of transglutaminase 2 contribute differentially to fibroblast wound healing responses. J Cell Sci 117:3389-403, 2004
20. Fesus L, Piacentini M: Transglutaminase 2: an enigmatic enzyme with diverse functions. Trends Biochem Sci 27:534-9, 2002
21. Akimov S S, Belkin A M: Cell surface tissue transglutaminase is involved in adhesion and migration of monocytic cells on fibronectin. Blood 98:1567-76, 2001
22. Akimov S S, Krylov D, Fleischman L F, et al: Tissue transglutaminase is an integrin-binding adhesion coreceptor for fibronectin. J Cell Biol 148:825-38, 2000
23. Verderio E A, Telci D, Okoye A, et al: A novel RGD-independent cel adhesion pathway mediated by fibronectin-bound tissue transglutaminase rescues cells from anoikis. J Biol Chem 278:42604-14, 2003
24. Piredda L, Farrace M G, Lo Bello M, et al: Identification of ‘tissue’ transglutaminase binding proteins in neural cells committed to apoptosis. Faseb J 13:355-64, 1999
25. Kondapaka S B, Singh S S, Dasmahapatra G P, et al: Perifosine, a novel alkylphospholipid, inhibits protein kinase B activation. Mol Cancer Ther 2:1093-103, 2003
26. Facchiano F, Facchiano A, Facchiano A M: The role of transglutaminase-2 and its substrates in human diseases. Front Biosci 11:1758-73, 2006
27. Yuan Z Q, Sun M, Feldman R I, et al: Frequent activation of AKT2 and induction of apoptosis by inhibition of phosphoinositide-3-O H kinase/Akt pathway in human ovarian cancer. Oncogene 19:2324-30, 2000
28. Akimov S S, Belkin A M: Cell-surface transglutaminase promotes fibronectin assembly via interaction with the gelatin-binding domain of fibronectin: a role in TGFbeta-dependent matrix deposition. J Cell Sci 114:2989-3000, 2001
29. Belkin A M, Tsurupa G, Zemskov E, et al: Transglutaminase-mediated oligomerization of the fibrin(ogen) {alpha}C-domains promotes integrin-dependent cell adhesion and signaling. Blood, 2005
30. Kaartinen M T, El-Maadawy S, Rasanen N H, et al: Tissue transglutaminase and its substrates in bone. J Bone Miner Res 17:2161-73, 2002
31. Aeschlimann D, Paulsson M: Cross-linking of laminin-nidogen complexes by tissue transglutaminase. A novel mechanism for basement membrane stabilization. J Biol Chem 266:15308-17, 1991
32. Jones R A, Kotsakis P, Johnson T S, et al: Matrix changes induced by transglutaminase 2 lead to inhibition of angiogenesis and tumor growth. Cell Death Differ, 2005
33. Iacobuzio-Donahue C A, Ashfaq R, Maitra A, et al: Highly expressed genes in pancreatic ductal adenocarcinomas: a comprehensive characterization and comparison of the transcription profiles obtained from three major technologies. Cancer Res 63:8614-22, 2003
34. Derynck R, Zhang Y E: Smad-dependent and Smad-independent pathways in TGF-beta family signalling. Nature 425:577-84, 2003
35. Martinet N, Bonnard L, Regnault V, et al: In vivo transglutaminase type 1 expression in normal lung, preinvasive bronchial lesions, and lung cancer. Am J Respir Cell Mol Biol 28:428-35, 2003
36. Grigoriev M Y, Suspitsin E N, Togo A V, et al: Tissue transglutaminase expression in breast carcinomas. J Exp Clin Cancer Res 20:265-8, 2001
37. Verma A, Wang H, Manavathi B, et al: Increased expression of tissue transglutaminase in pancreatic ductal adenocarcinoma and its implications in drug resistance and metastasis. Cancer Res 66:10525-33, 2006
38. Hwang J Y, Mangala L S, Fok J Y, et al: Clinical and biological significance of tissue transglutaminase in ovarian carcinoma. Cancer Res 68:5849-58, 2008
39. Jeong J H, Cho B C, Shim H S, et al: Transglutaminase 2 expression predicts progression free survival in non-small cell lung cancer patients treated with epidermal growth factor receptor tyrosine kinase inhibitor. J Korean Med Sci 28:1005-14, 2013
40. Mehta K, Lopez-Berestein G, Moore W T, et al: Interferon-gamma requires serum retinoids to promote the expression of tissue transglutaminase in cultured human blood monocytes. J Immunol 134:2053-6, 1985
41. Cao L, Petrusca D N, Satpathy M, et al: Tissue Transglutaminase Protects Epithelial Ovarian Cancer Cells from Cisplatin Induced Apoptosis by Promoting Cell Survival Signaling. Carcinogenesis, 2008
42. Mann A P, Verma A, Sethi G, et al: Overexpression of Tissue Transglutaminase Leads to Constitutive Activation of Nuclear Factor-{kappa}B in Cancer Cells: Delineation of a Novel Pathway. Cancer Res 66:8788-95, 2006
43. Mehta K, Fok J, Miller F R, et al: Prognostic significance of tissue transglutaminase in drug resistant and metastatic breast cancer. Clin Cancer Res 10:8068-76, 2004
44. Kumar A, Xu J, Brady S, et al: Tissue transglutaminase promotes drug resistance and invasion by inducing mesenchymal transition in mammary epithelial cells. PLoS One 5:e13390, 2010
45. Kumar A, Gao H, Xu J, et al: Evidence that aberrant expression of tissue transglutaminase promotes stem cell characteristics in mammary epithelial cells. PLoS One 6:e20701, 2011
46. Kerr C, Szmacinski H, Fisher M L, et al: Transamidase site-targeted agents alter the conformation of the transglutaminase cancer stem cell survival protein to reduce GTP binding activity and cancer stem cell survival. Oncogene 36:2981-2990, 2017
47. Sullivan K E, Rojas K, Cerione R A, et al: The stem cell/cancer stem cell marker ALDH1A3 regulates the expression of the survival factor tissue transglutaminase, in mesenchymal glioma stem cells. Oncotarget 8:22325-22343, 2017
48. Condello S, Cao L, Matei D: Tissue transglutaminase regulates beta-catenin signaling through a c-Src-dependent mechanism. FASEB J 27:3100-12, 2013
49. Verma A, Guha S, Diagaradjane P, et al: Therapeutic significance of elevated tissue transglutaminase expression in pancreatic cancer. Clin Cancer Res 14:2476-83, 2008
50. Singh U S, Kunar M T, Kao Y L, et al: Role of transglutaminase II in retinoic acid-induced activation of RhoA-associated kinase-2. EMBO J 20:2413-23, 2001
51. Kumar A, Xu J, Sung B, et al: Evidence that GTP-binding domain but not catalytic domain of transglutaminase 2 is essential for epithelial-to-mesenchymal transition in mammary epithelial cells. Breast Cancer Res 14:R4, 2012
52. Satpathy M, Shao M, Emerson R, et al: Tissue transglutaminase regulates matrix metalloproteinase-2 in ovarian cancer by modulating cAMP-response element-binding protein activity. J Biol Chem 284:15390-9, 2009
53. Lee J, Condello S, Yakubov B, et al: Tissue Transglutaminase Mediated Tumor-Stroma Interaction Promotes Pancreatic Cancer Progression. Clin Cancer Res 21:4482-93, 2015
54. Lee J, Yakubov B, Ivan C, et al: Tissue Transglutaminase Activates Cancer-Associated Fibroblasts and Contributes to Gemcitabine Resistance in Pancreatic Cancer. Neoplasia 18:689-698, 2016
55. Choi K, Siegel M, Piper J L, et al: Chemistry and biology of dihydroisoxazole derivatives: selective inhibitors of human transglutaminase 2. Chem Biol 12:469-75, 2005
56. Zhou L P, Yang L H, Tilton S, et al: Development of a high throughput equilibrium solubility assay using miniaturized shake-flask method in early drug discovery. Journal of Pharmaceutical Sciences 96:3052-3071, 2007
57. Khanna M, Chelladurai B, Gavini A, et al: Targeting ovarian tumor cell adhesion mediated by tissue transglutaminase. Mol Cancer Ther 10:626-36, 2011
58. Villa S R, Mishra R K, Zapater J L, et al: Homology modeling of FFA2 identifies novel agonists that potentiate insulin secretion. J Investig Med 65:1116-1124, 2017
59. Mishra R K, Shum A K, Platanias L C, et al: Discovery and characterization of novel small-molecule CXCR4 receptor agonists and antagonists. Sci Rep 6:30155, 2016
60. Zhu J, Mishra R K, Schiltz G E, et al: Virtual High-Throughput Screening To Identify Novel Activin Antagonists. J Med Chem 58:5637-48, 2015
61. Mishra R K, Wei C, Hresko R C, et al: In Silico Modeling-based Identification of Glucose Transporter 4 (GLUT4)-selective Inhibitors for Cancer Therapy. J Biol Chem 290:14441-53, 2015
62. Dong R, Qiang W, Guo H, et al: Histologic and molecular analysis of patient derived xenografts of high-grade serous ovarian carcinoma. J Hematol Oncol 9:92, 2016
63. Zhang S, Balch C, Chan M W, et al: Identification and characterization of ovarian cancer-initiating cells from primary human tumors. Cancer Res 68:4311-20, 2008
64. Gillan L, Matei D, Fishman D A, et al: Periostin secreted by epithelial ovarian carcinoma is a ligand for alpha(V)beta(3) and alpha(V)beta(5) integrins and promotes cell motility. Cancer Res 62:5358-64, 2002
65. Nurminskaya M V, Belkin A M: Cellular functions of tissue transglutaminase. Int Rev Cell Mol Biol 294:1-97, 2012
66. Zemskov E A, Loukinova E, Mikhailenko I, et al: Regulation of platelet-derived growth factor receptor function by integrin-associated cell surface transglutaminase. J Biol Chem 284:16693-703, 2009
67. Zemskov E A, Mikhailenko I, Smith E P, et al: Tissue transglutaminase promotes PDGF/PDGFR-mediated signaling and responses in vascular smooth muscle cells. J Cell Physiol 227:2089-96, 2012

### Example 3—Exemplary Compounds

| Molecule Name | Structure |
|---|---|
| NUCC-0203016 | 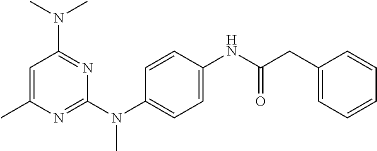 [SMILES: Cc1cc(N(C)C)nc(N(C)c2ccc(NC(=O)Cc3ccccc3)cc2)n1] |
| NUCC-0203015 | 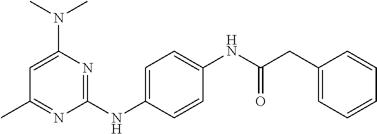 [SMILES: Cc1cc(N(C)C)nc(Nc2ccc(NC(=O)Cc3ccccc3)cc2)n1] |
| NUCC-0203014 | 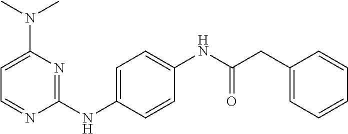 [SMILES: CN(C)c1ccnc(Nc2ccc(NC(=O)Cc3ccccc3)cc2)n1] |
| NUCC-0203013 | 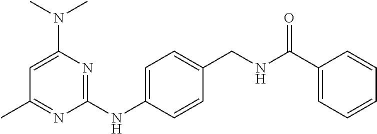 [SMILES: Cc1cc(N(C)C)nc(Nc2ccc(CNC(=O)c3ccccc3)cc2)n1] |
| NUCC-0203012 | 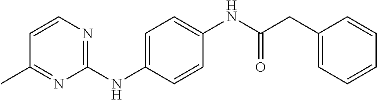 [SMILES: Cc1ccnc(Nc2ccc(NC(=O)Cc3ccccc3)cc2)n1] |
| NUCC-0203011 | 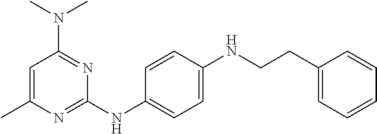 [SMILES: Cc1cc(N(C)C)nc(Nc2ccc(NCCc3ccccc3)cc2)n1] |
| NUCC-0203010 | 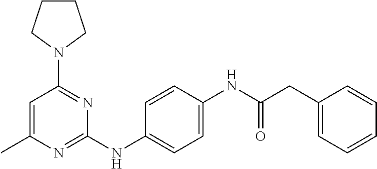 [SMILES: Cc1cc(N2CCCC2)nc(Nc2ccc(NC(=O)Cc3ccccc3)cc2)n1] |
| NUCC-0203009 | 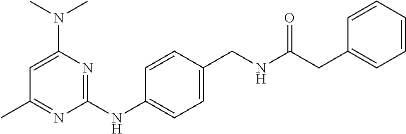 [SMILES: Cc1cc(N(C)C)nc(Nc2ccc(CNC(=O)Cc3ccccc3)cc2)n1] |
| NUCC-0203008 | 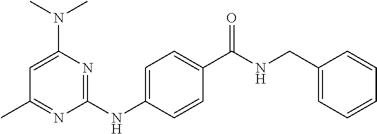 [SMILES: Cc1cc(N(C)C)nc(Nc2ccc(C(=O)NCc3ccccc3)cc2)n1] |
| NUCC-0203007 | 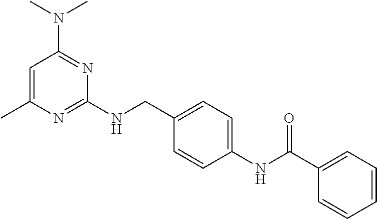 [SMILES: Cc1cc(N(C)C)nc(NCc2ccc(NC(=O)c3ccccc3)cc2)n1] |
| NUCC-0203006 | 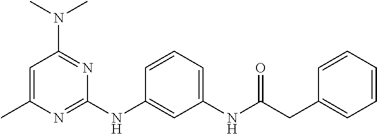 [SMILES: Cc1cc(N(C)C)nc(Nc2cccc(NC(=O)Cc3ccccc3)c2)n1] |
| NUCC-0203005 | 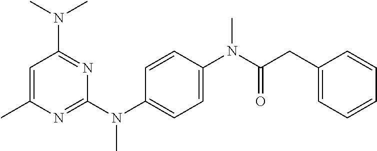 [SMILES: Cc1cc(N(C)C)nc(N(C)c2ccc(N(C)C(=O)Cc3ccccc3)cc2)n1] |
| NUCC-0203004 | 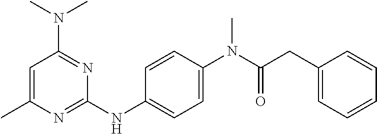 [SMILES: Cc1cc(N(C)C)nc(Nc2ccc(N(C)C(=O)Cc3ccccc3)cc2)n1] |
| NUCC-0203003 | 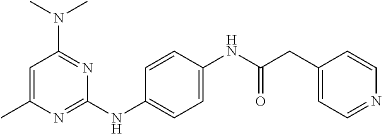 [SMILES: Cc1cc(N(C)C)nc(Nc2ccc(NC(=O)Cc3ccncc3)cc2)n1] |
| NUCC-0203002 | 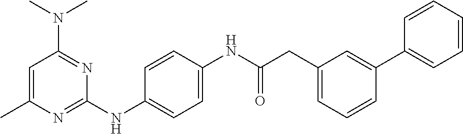 [SMILES: Cc1cc(N(C)C)nc(Nc2ccc(NC(=O)Cc3cccc(-c4ccccc4)c3)cc2)n1] |
| NUCC-0203001 | 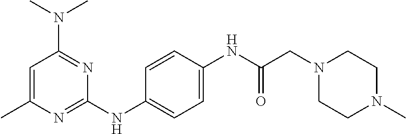 |
| NUCC-0203000 | 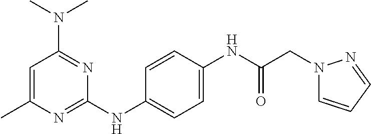 [SMILES: Cc1cc(N(C)C)nc(Nc2ccc(NC(=O)Cn3cccn3)cc2)n1] |
| NUCC-0202999 | 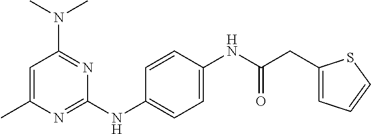 [SMILES: Cc1cc(N(C)C)nc(Nc2ccc(NC(=O)Cc3cccs3)cc2)n1] |
| NUCC-0202998 | 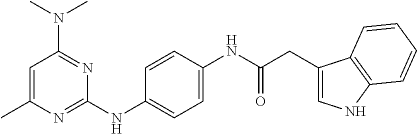 [SMILES: Cc1cc(N(C)C)nc(Nc2ccc(NC(=O)Cc3c[nH]c4ccccc34)cc2)n1] |
| NUCC-0202997 | 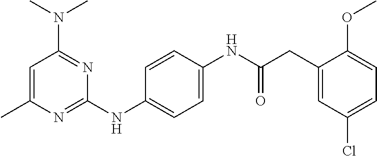 |

### Example 4—Inhibition of the Interaction Between Tissue Transglutaminase 2 (TG2) and Fibronectin

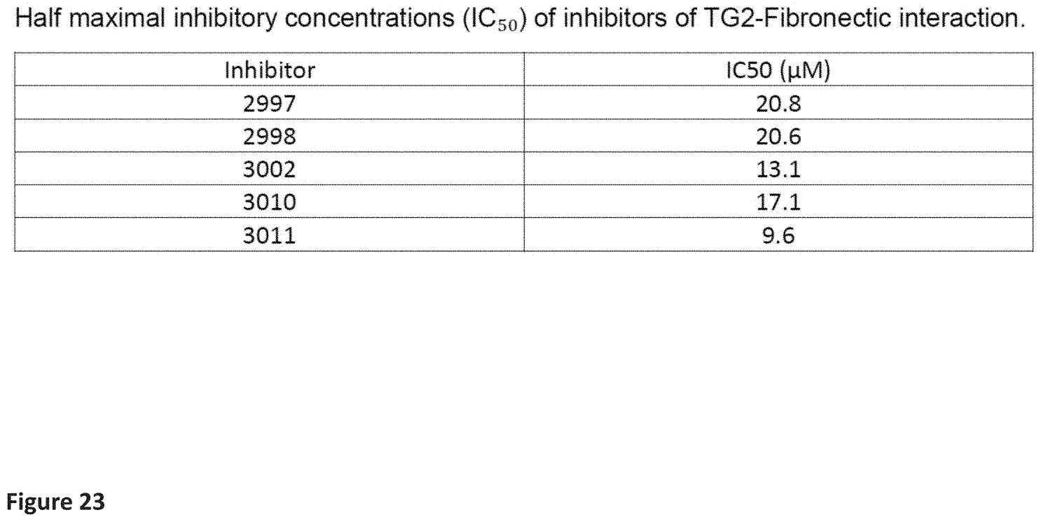

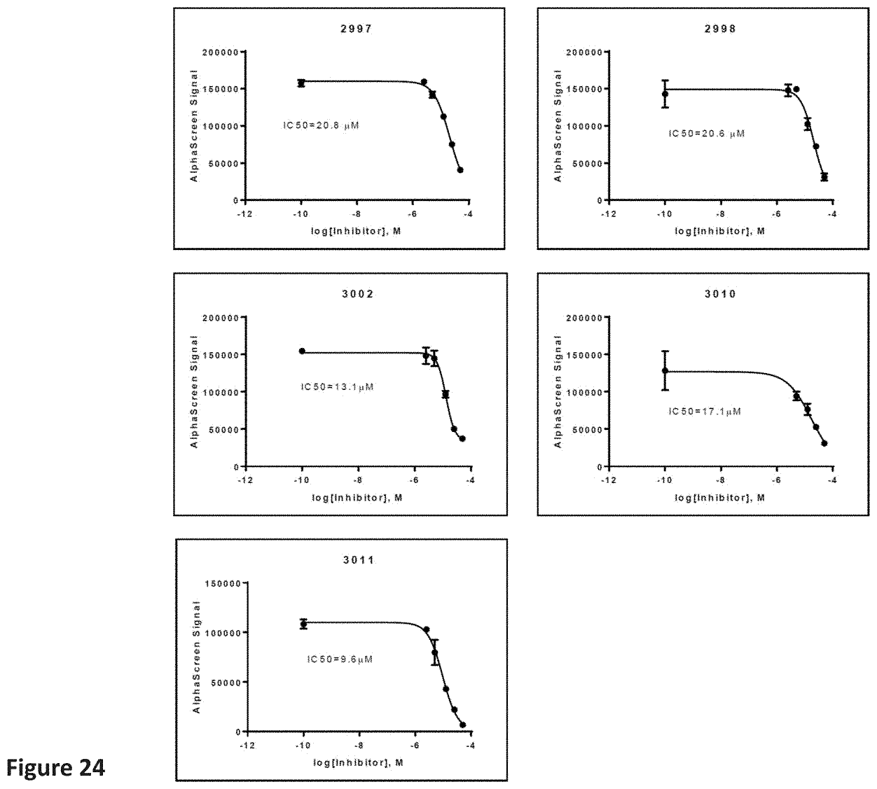

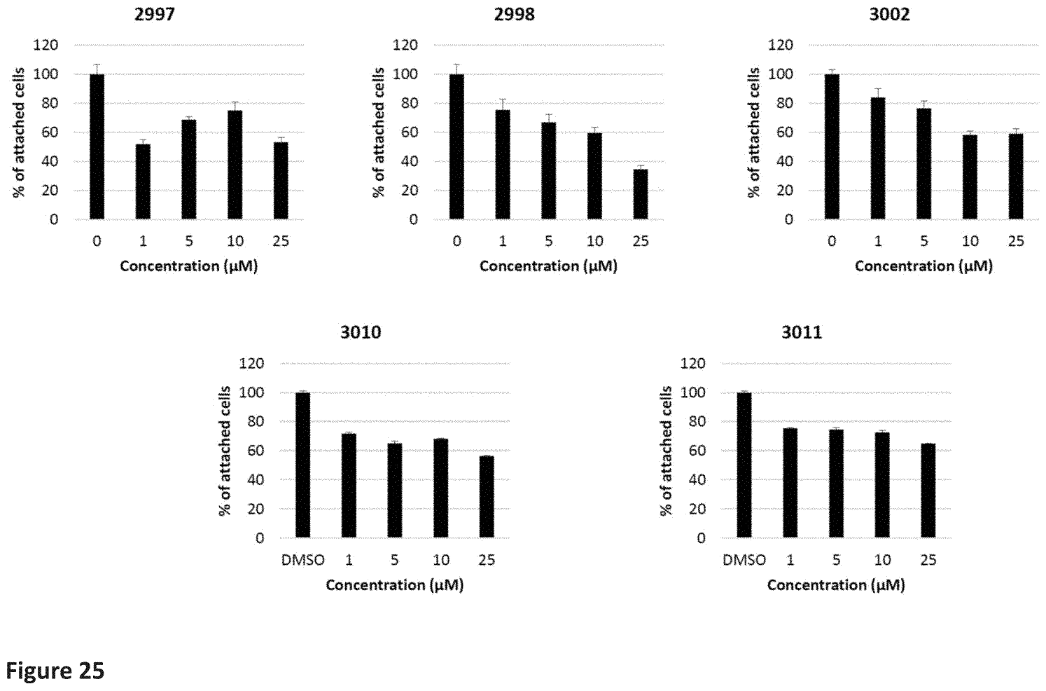

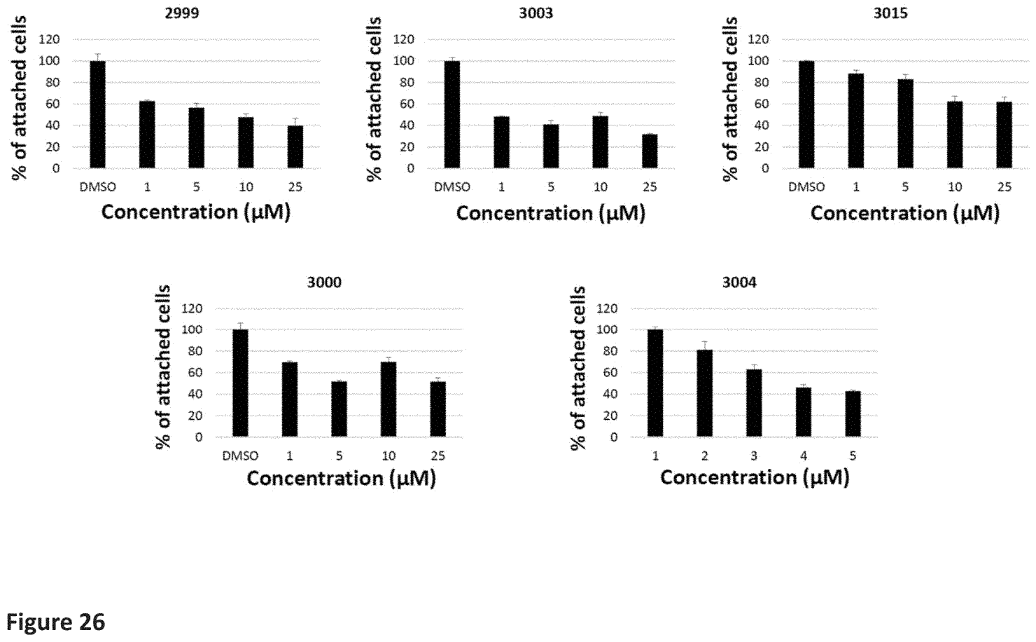

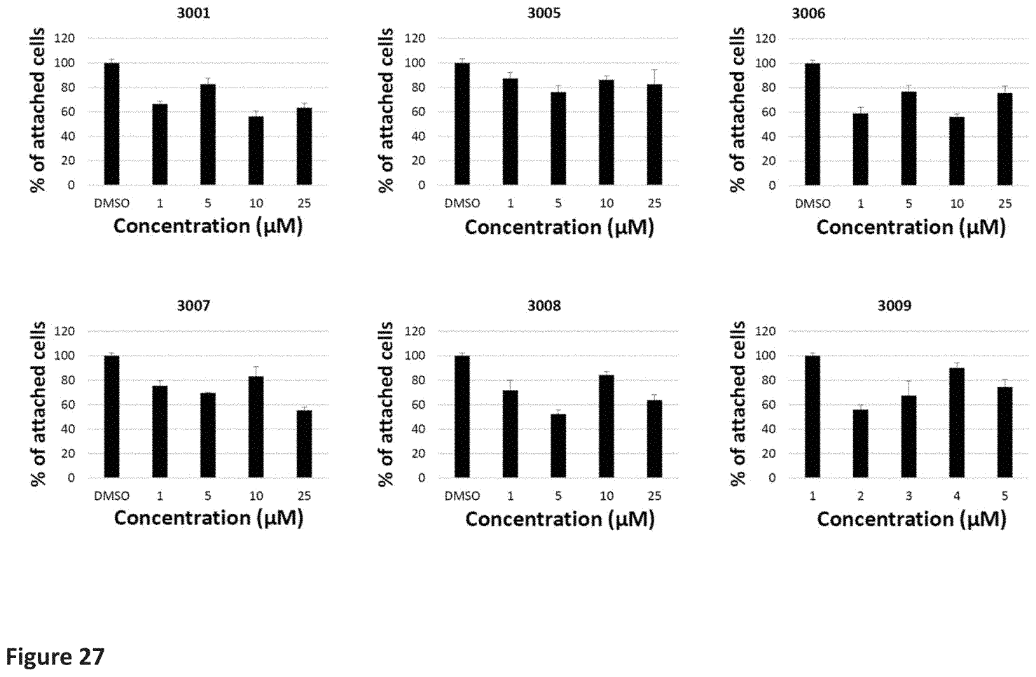

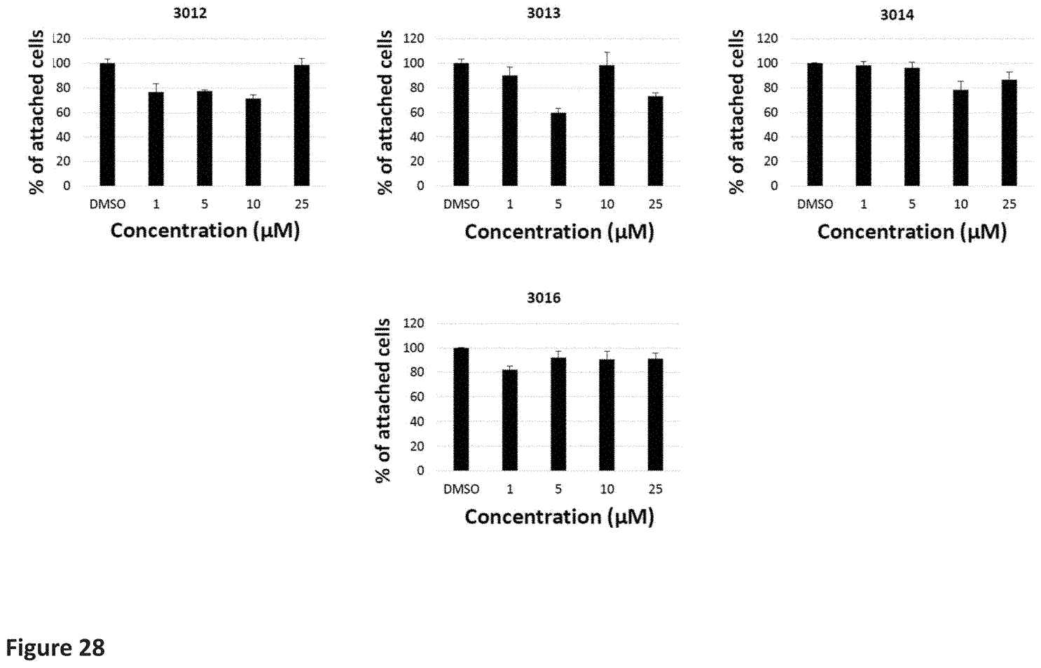

The compounds of Example 3 were tested for inhibition of the interaction between tissue transglutaminase 2 (TG2) and fibronectin using the methods described in Examples 1 and 2. (See Results presented in FIGS. 23-28).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

### Example 5—Supplementary Material for Example 1

Inhibitors Synthesis

Materials. Dimethylformamide (DMF), methanol (MeOH) and ethyl acetate (EtOAc) were from Fisher Scientific. N,N-Diisopropylethylamine (DIPEA),p-phenylenediamine, 3-dimethylamino-benzoicacid, 3-methoxylbenzoicacid, 3,4-dichlorobenzoicacid, 4-phenylacetic acid, 2-naphthoic acid, 3-bromobenzoic acid, and propylphosphonic anhydride (T3P) were from Aldrich.

Instrumentation. HPLC purification was carried out on a Waters Breeze HPLC system equipped with a Waters Atlantis dC18 column (10 μm, 19 mm×100 mm). NMR spectra were recorded on a Bruker Fourier 300 NMR spectrometer. Mass spectra (ESI) was recorded on an Agilent 6130 Quadrupole MS detector. HPLC purity analysis was carried out on an Agilent 1200 analytic HPLC system, equipped with a Phenomenex Kinetex 2.6u XB-C18 column (2.5 μm, 4.6 mm×50 mm), eluted with a 0-100% MeOH-H₂O with 0.1% (v/v) TFA at 0.8 mL/min flowrate.

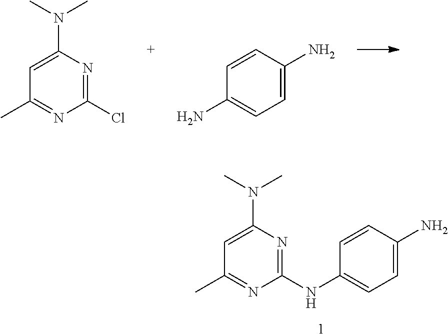

Synthesis of compound 1. Crimidine (3.42 g, 20 mmole) and p-phenylenediamine (2.16 g, 20 mmole) were dissolved in 20 mL DMF. The mixture was radiated with microwave using a Biotage Isabela Microwave System for 15 min at 150° C. The solution was cooled down to room temperature and the precipitate was collected by filtration and washed with ethyl acetate to obtain the intermediate 1. The compound 1 was used for the next step without further purification.

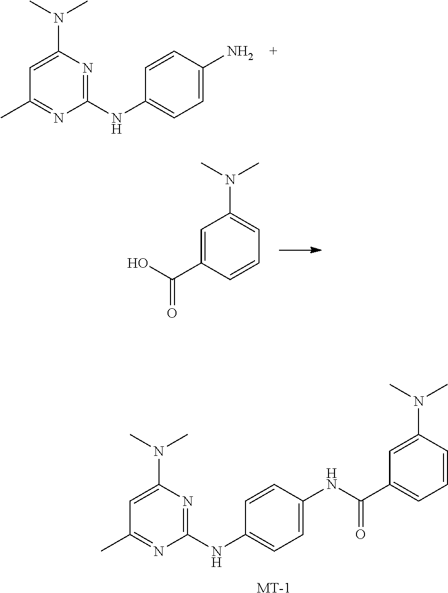

Synthesis of compound MT-1. The compound 1 (243 mg, 1 mmole), 3-(dimethylamino)benzoicacid (165 mg, 1 mmole) were dissolved in 5 mL DMF. DIPEA (500 uL) were added to the solution, followed by T3P in DMF (˜50%, 1 mL). The mixture was stirred at room temperature for 180 min. The crude product was obtained by removing solvent in vacuo and purified by HPLC. The pure compound (120 mg, 31% yield) was obtained. ¹H NMR (300 μMHz, DMSO-d₆): δ=9.97 (s, 1H), 8.96 (s, 1H), 7.76-7.65 (d, 2H, J=9.1 Hz), 7.65-7.51 (d, 2H, J=9.1 Hz), 7.38-7.21 (m, 3H), 6.95-6.82 (m, 1H), 5.99-5.95 (m, 1H), 3.05 (s, 6H), 2.96 (s, 6H), 2.18 (s, 3H). MS (ESI): calcd for [M]: 390, found [M+H]⁺: 391. Purity: >95% (UV, k=254 nm).

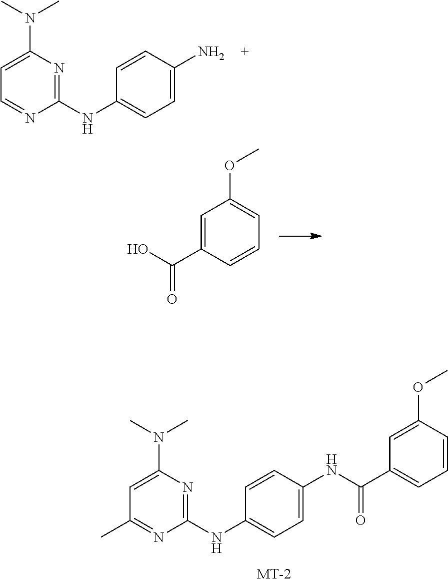

Synthesis of compound MT-2. The compound 1 (486 mg, 2 mmole), 3-methoxylbenzoicacid (304 mg, 2 mmole) were dissolved in 10 mL DMF. DIPEA (2 mL) were added to the solution, followed by T3P in DMF (˜50%, 2 mL). The mixture was stirred at room temperature for 120 min. The crude product was obtained by removing solvent in vacuo and purified by HPLC. The pure compound (218 mg, 29% yield) was obtained. ¹H NMR (300 μMHz, DMSO-d₆): δ=10.06 (s, 1H), 8.98 (s, 1H), 7.95 (s, 1H), 7.79-7.66 (d, 2H, J=9.1 Hz), 7.66-7.55 (d, 2H, J=9.1 Hz), 7.55-7.31 (m, 3H), 7.09-7.03 (m, 1H), 6.01-5.95 (m, 1H), 3.79 (s, 3H), 3.05 (s, 6H), 2.96 (s, 6H), 2.19 (s, 3H)(FigureS2). MS (ESI): calcd for [M]: 377, found [M+H]⁺: 378. Purity: >95% (UV, λ=254 nm).

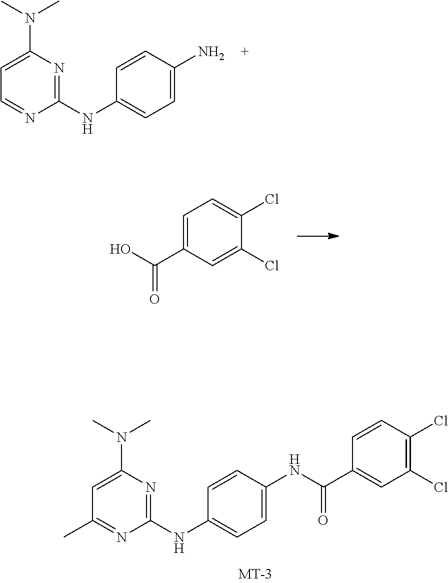 [SMILES: CN(C)c1ccnc(Nc2ccc(N)cc2)n1 ; O=C(O)c1ccc(Cl)c(Cl)c1 ; Cc1cc(N(C)C)nc(Nc2ccc(NC(=O)c3ccc(Cl)c(Cl)c3)cc2)n1]

Synthesis of compound MT-3. The compound 1 (243 mg, 1 mmole), 3,4-dichlorobenzoicacid (191 mg, 1 mmole) were dissolved in 5 mL DMF. DIPEA (0.5 mL) were added to the solution, followed by T3P in DMF (˜50%, 2 mL). The mixture was stirred at room temperature for 18 hours. The crude product was obtained by removing solvent in vacuo and purified by HPLC. The pure compound (156 mg, 38% yield) was obtained. ¹H NMR (300 μMHz, DMSO-d₆): δ=10.25 (s, 1H), 9.01 (s, 1H), 8.24-8.16 (m, 1H), 7.95-7.51 (m, 6H), 5.98 (s, 1H), 3.05 (s, 6H), 2.19 (s, 3H). MS (ESI): calcd for [M]: 415, found [M+H]⁺: 416. Purity: >95% (UV, λ=254 nm).

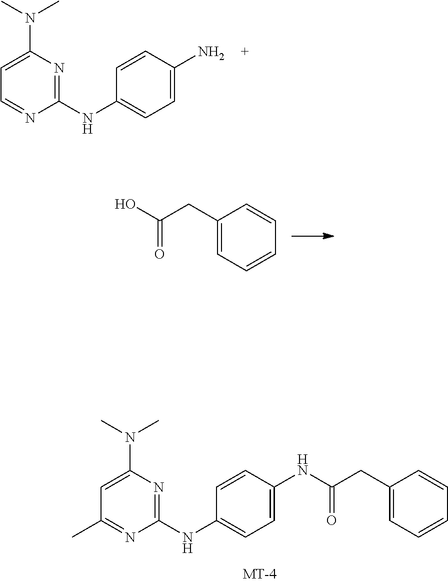 [SMILES: CN(C)c1ccnc(Nc2ccc(N)cc2)n1 ; O=C(O)Cc1ccccc1 ; Cc1cc(N(C)C)nc(Nc2ccc(NC(=O)Cc3ccccc3)cc2)n1]

Synthesis of compound MT-4. The compound 1 (364 mg, 1.5 mmole), 4-phenylacetic acid (204 mg, 1.5 mmole) were dissolved in 5 mL DMF. DIPEA (1 mL) were added to the solution, followed by T3P in DMF (˜50%, 3 mL). The mixture was stirred at room temperature for 30 minutes. The crude product was obtained by removing solvent in vacuo and purified by HPLC. The pure compound (162 mg, 30% yield) was obtained. ¹H NMR (300 μMHz, DMSO-d₆): δ=10.00 (s, 1H), 8.92 (s, 1H), 7.72-7.59 (d, 2H, J=9.0 Hz), 7.49-7.39 (d, 2H, J=9.0 Hz), 7.38-7.11 (m, 5H), 5.95 (s, 1H), 3.59 (s, 2H), 3.02 (s, 6H), 2.16 (s, 3H). MS (ESI): calcd for [M]: 361, found [M+H]⁺: 362. Purity: >95% (UV, λ=254 nm).

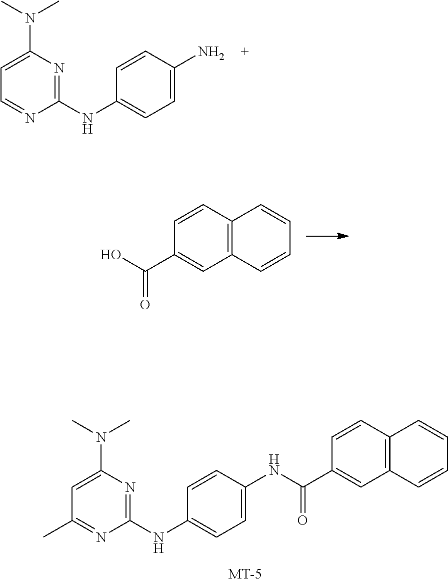 [SMILES: CN(C)c1ccnc(Nc2ccc(N)cc2)n1 ; O=C(O)c1ccc2ccccc2c1 ; Cc1cc(N(C)C)nc(Nc2ccc(NC(=O)c3ccc4ccccc4c3)cc2)n1]

Synthesis of compound MT-5. The compound 1 (243 mg, 1 mmole), 2-naphthoic acid (172 mg, 1 mmole) were dissolved in 5 mL DMF. DIPEA (0.5 mL) were added to the solution, followed by T3P in DMF (˜50%, 1 mL). The mixture was stirred at room temperature for 60 minutes. The crude product was obtained by removing solvent in vacuo and purified by HPLC. The pure compound (112 mg, 28% yield) was obtained. ¹H NMR (300 μMHz, DMSO-d₆): δ=10.30 (s, 1H), 9.00 (s, 1H), 8.56 (s, 1H), 8.11-7.92 (m, 4H), 7.81-7.53 (m, 6H), 5.98 (s, 1H), 3.05 (s, 6H), 2.19 (s, 3H). MS (ESI): calcd for [M]: 397, found [M+H]⁺: 398. Purity: >95% (UV, λ=254 nm).

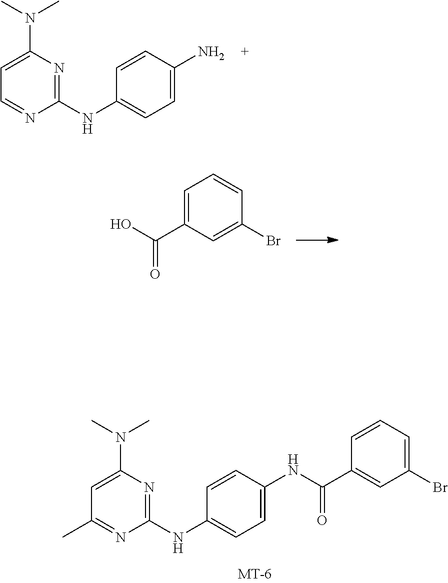 [SMILES: CN(C)c1ccnc(Nc2ccc(N)cc2)n1 ; O=C(O)c1cccc(Br)c1 ; Cc1cc(N(C)C)nc(Nc2ccc(NC(=O)c3cccc(Br)c3)cc2)n1]

Synthesis of compound MT-6. The compound 1 (243 mg, 1 mmole), 3-bromobenzoic acid (201 mg, 1 mmole) were dissolved in 5 mL DMF. DIPEA (0.5 mL) were added to the solution, followed by T3P in DMF (˜50%, 2 mL). The mixture was stirred at room temperature for 30 minutes. The crude product was obtained by removing solvent in vacuo and purified by HPLC. The pure compound (130 mg, 30% yield) was obtained. ¹H NMR (300 μMHz, DMSO-d₆): δ=10.21 (s, 1H), 9.00 (s, 1H), 8.12 (s, 1H), 7.98-7.84 (m, 1H), 7.84-7.42 (m, 6H), 5.98 (s, 1H), 3.05 (s, 6H), 2.18 (s, 3H). MS (ESI): calcd for [M]: 425, found [M+H]⁺: 426. Purity: >95% (UV, λ=254 nm).

Solubility Assay

Solubility was measured in a 384-well plate using a Synergy HTX plate reader. Starting with a 50 mM DMSO stock solution of test compounds, dilutions were made from 1 to 500 μM final concentrations using acetonitrile, keeping the final DMSO concentration at 2%. UV absorption was measured at 254 nm and absorbance versus concentration was plotted to give a standard curve. A DMSO stock of test compound was also diluted with pH=7.4 PBS buffer to give a final concentration of 250 μM containing 2% DMSO. This mixture was stirred on a plate shaker for 1 hr after which it was centrifuged at 2500 rpm for 3 min. 50 μL of the supernatant was removed, transferred to a 384-well plate, and the UV absorbance at 254 nm was measured. Absorbance of the test compound in PBS buffer was analyzed with the standard curve to determine solubility.

| Compound | Solubility | |
|---|---|---|
| TG53 | <5 | μM |
| MT4 | 91.5 | μM |
| MT6 | 6.4 | μM |

Enzyme-Linked Immunosorbent Assay (ELISA)

ELISA measured the interaction between recombinant His tagged TG2 (rTG2-His) and the biotinylated 42 kD fragment of FN (FN42) which were expressed and purified, as previously described (Yakubov et al., 2014). Briefly, a mixture of 10 nMrTG2-His and selected SMIs or DMSO was added to 96 well plates pre-coated with monoclonal anti-His antibody (Roche, Indianapolis, Ind.; 1:6000). The mixture was incubated for 1 h at RT with 5 nM biotinylated FN42. After washing, wells were incubated with streptavidin-HRP (Cell Signaling, #3999; 1:2000). The reaction was developed with 3,3′,5,5′tetramethylbenzidine (TMB) substrate (BD Biosciences, #555214) and the optical density at 405 nm was measured by using a BioTek microplate reader (Winooski, Vt., USA). Data shown are means±SEM of two independent experiments (n=6).

TG2 Enzymatic Activity Assay

To measure TG2 enzymatic activity, the formation of hydroxamate from Nu-CBZ-glutaminyl-glycine and hydroxylamine was measured by using a colorimetric assay as previously described(Folk and Cole, 1966). In brief, 5 μg of purified TG2 (Sigma #T5398) was incubated with Nu-CBZ-glutaminyl-glycine and hydroxylamine in the presence of Ca²⁺ at 37° C. and TGase activity was measured as the amount of hydroxamate generated. The colorimetric reaction was measured at 525 nm in an xMark Bio-Rad microplate reader. Selected inhibitors were used in concentrations ranging from 1 to 30 μM. All assays were performed in triplicates. Data are presented as means±SD.

CCK-8 Cell Proliferation Assays

Cell proliferation was assessed by using the CCK-8 assay and following the manufacturer's specifications (Dojindo, #CK04-05). Absorbance at 450 nm was quantified using a BioTek plate reader. Sigmoidal regression curves were generated to calculate IC50 values by using GraphPad Prism Software.

Solid Phase Adhesion Assay

Cells labeled with calcein AM (Life Technologies, #L3224) were seeded at a density of 4×10⁴ cells in 96-well plates pre-coated with FN (5 μg/mL, Sigma, #F1141), and blocked using 1% BSA (Sigma, #A9647) in PBS in the presence of selected compounds diluted at the specified concentrations, in serum free media. After 45 minutes, unattached cells were washed using PBS containing 1 mM MgCl₂(Sigma, #M8266) and adherent cells were quantified in a SpectraMaxGeminiXS fluorescence plate reader (Molecular Devices, Palo Alto, Calif.) at 530 nm wavelength. Experiments were performed inquadruplicate and repeated at least twice.

Wound-Healing Assay

Wound Healing was Performed in 12-Well Plates Coated with 1 μg/Ml FN (Sigma, #1141) overnight at 4° C. OC cells were plated at a density of 2×10⁵ cells/well in media containing 2% FBS. Under these conditions cells adhered on substrate, but cell proliferation was minimized. After 16 hours, the cell monolayer was scraped in straight line. Cell debris was removed and serum free media containing selected SMIs or control was replenished. For consistency during image acquisition, a reference point was marked close to the scratch mark and the plate was imaged under phase-contrast microscope at 0, 2, 6, 12 and 24 hours. Wound closure was assessed as the distance between the wound edges, calculated and quantified using the Image Pro-Express software. The percentage of wound closure was evaluated using the formula (wound size at 24 hours/initial wound size)×100. The rate of migration (μm/h) of OC cells into the wound space was evaluated as the distance difference between wound edges at 0 and 24 h, divided by 24. The experiments were performed in duplicates.

Early Attachment Assay

OVCAR5 cells were seeded(1×10⁵ cells/well) in 6-well plates (Denville Scientific) and treated for 72 hrs with 1 μM MT-4, TG53 or vehicle control (DMSO). After drug exposure, cells were detached using Cellstripper (cellgro, #25-056-CI, CORNING, Manassas, Va., USA), centrifuged, and counted. Before seeding for either confocal microscopy or western blotting, they were incubated in serum free media in the presence of drugs for 15-90 minutes, as stated in the figure legends.

Immunofluorescent (IF) Staining and Confocal Microscopy

In order to analyze cell adhesion complex proteins expression and localization upon early attachment, cells were treated as stated above and then seeded onto FN-coated cover slips for 30 minutes at 37° C. Samples were fixed in 4% p-formaldehyde (PFA) and permeabilized with 0.2% Triton-X-100. After blocking, focal contacts were identified by using anti-vinculin antibodies, activation of integrins signaling pathway was assessed by antibodies against phosphorylated focal adhesion kinase (FAK), while actin cytoskeleton was labeled by Alexa Fluor 488-conjugated phalloidin (Life Technologies, #A12379). Nuclei were stained with 1:10000 Hoechst (Thermo Scientific) and specimens were mounted in Prolong Gold anti-fadereagent (Life Technologies). Cells were analyzed by laser scanning using a Nikon A1RGaAsPConfocal Laser Microscope. For co-localization analyses, SKOV3 cells were treated with DMSO (control) or TG53 (1 μM) and plated on FN-coated chamber slides (BD Biosciences) for 72 hrs. IF analysis was performed as previously described (Condello et al., 2013b). Briefly, after fixation, permeabilization and blocking, cells were incubated overnight with p-FAK (Y576/577), c-Src, p-Src (Y416), integrin β1 or TG2 primary antibodies in blocking buffer at 4° C., followed by 1 h incubation with Cy5-conjugated anti-mouse secondary antibody (1:500; Zymed Laboratories; South San Francisco, Calif., USA) or Alexa Fluor 488 anti-rabbit secondary antibody (1:1000; Molecular Probes, Eugene, Oreg., USA) at RT. Isotype-specific IgG was used as a negative control. Nuclei were visualized by 4′,6-diamidino-2-phenylindole (DAPI) staining (Vectashield; Vector Laboratories, Burlingame, Calif., USA). Analysis was done using an LSM 510 META confocal multiphoton microscope system (Carl Zeiss Inc., Thomwood, N.Y., USA) under UV excitation at 630 nm (for Cy5), 488 nm (for Alexa Fluor 488), and 340 nm (for DAPI) and using a 60× magnification objective. Colocalization percentage was calculated by normalizing the pixels corresponding to Cy5 or Alexa Fluor 488 emissions to DAPI pixels in the same cell, followed by merging area of green with area of red and determination of average of overlap areas. A Student' t test was calculated to determine statistical significance of differences between drug treated and DMSO control treated cells.

Western Blotting

Cells pre-treated as stated above were seeded in 60 mm dishes coated with 5 μg/mL FN and serially harvested at 15, 30, and 45 minutes, − or left unattached (0 min), in cold RIPA buffer. Lysates were separated by SDS-PAGE, transferred onto PVDF membranes (Bio-Rad), incubated with primary and secondary antibodies and developed by using SuperSignal™ West Femto Maximum Sensitivity Substrate and Pierce™ ECL Western Blotting Substrate, respectively (Thermo Scientific). Images were obtained by using the ImageQuant LAS 4000 mini-imager (GE Healthcare, Pittsburgh, Pa.). Densitometry analysis was performed using Quantity One software (Bio-Rad) and results were normalized to GAPDH loading controls. DMSO 0 min time point was considered as 1 for comparative analysis.

Colony and Sphere Forming Assays

After 72 h priming with either 1 μM MT-4, TG53 or vehicle (DMSO), 150 OVCAR5 cells were seeded in 60 mm dishes and treated or not with paclitaxel (PTX, Sigma). Colonies were allowed to form over 10 days, fixed in 4% PFA and stained with 0.4% crystal violet (MP Biomedicals). Colonies were counted, and analysis was performed using the ImageJ software (hggs:nggejnib_ovI).Spheroid cultures were carried out as previously described(Condello et al., 2015). Briefly, OC cells were resuspended in Mammocult complete medium (StemCell Technologies) supplemented with Mammocult Proliferation Supplements, heparin and hydrocortisone (StemCell Technologies) and seeded at a concentration of 10,000 cells/well in 96-well ultra-low attachment plates (Corning, Corning, N.Y., USA). After treatment with DMSO (control) or TG53 (1 μM) for 7 days, spheroids were counted and quantified by the CCK-8 assay. All assays were performed in four replicates and were repeated. For priming before PTX treatment, cells were manipulated as described for CFU assay and then seeded in 6-well low-attachment plate (Corning, #3473) at 7000 cells/mL/well and allowed to form spheroids in complete Mammocult media. Spheres were counted after ten days from 4 replicate wells. Two independent experiments were performed.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A compound of a formula: 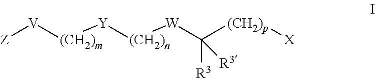
or a salt or solvate thereof,
wherein:
Z is pyrimidinyl or 5,7-diazaindolinyl and Z optionally is substituted at one or more ring positions with alkyl, alkoxy, amino, alkylamino, dialkylamino, heteroaryl, or halo;
V is NR¹ or O, wherein R¹ is hydrogen or alkyl;
m is selected from 0-6;
Y is an aryl group, a diaryl group, or a heteroaryl group, and Y optionally is substituted at one or more ring positions with haloalkyl;
n is selected from 0-6;
W is NR² or CH, wherein R² is hydrogen or alkyl;
R³ and R^{3′} are hydrogen or R³ and R^{3′} together form oxo;
p is selected from 0-6; and
X is an aryl group or a heteroaryl group, and X optionally is substituted at one or more ring positions with one or more of alkyl, alkoxy, halo; amino; alkylamino, or aryl; and
optionally with the proviso that if m is 0, n is 0, and p is 1, then X is not N-imidazyl or indol-3-yl; and
optionally with the proviso that if m is 0, n is 0, and p is 1, then Z is not 4-dimethylamino-pyrimidin-2-yl or 4-pyrrolidin-1-yl-pyrimidin-2-yl.

2. The compound of claim 1 having a formula Ia:

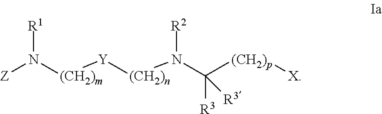

3. The compound of claim 1 having a formula Ib: 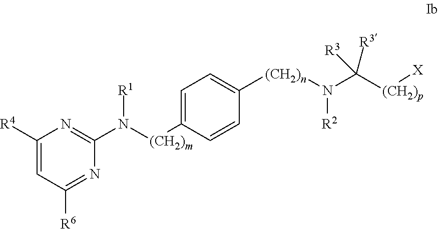
wherein:
R⁴ is hydrogen or R⁴ has a formula, 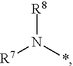
wherein R⁷ and R⁸ independently are hydrogen, alkyl, or a moiety comprising a biotinyl group or R⁷ and R⁸ together form linked alkyl; and
R⁶ is hydrogen or alkyl.

4. The compound of claim 1, wherein X has a formula selected from:

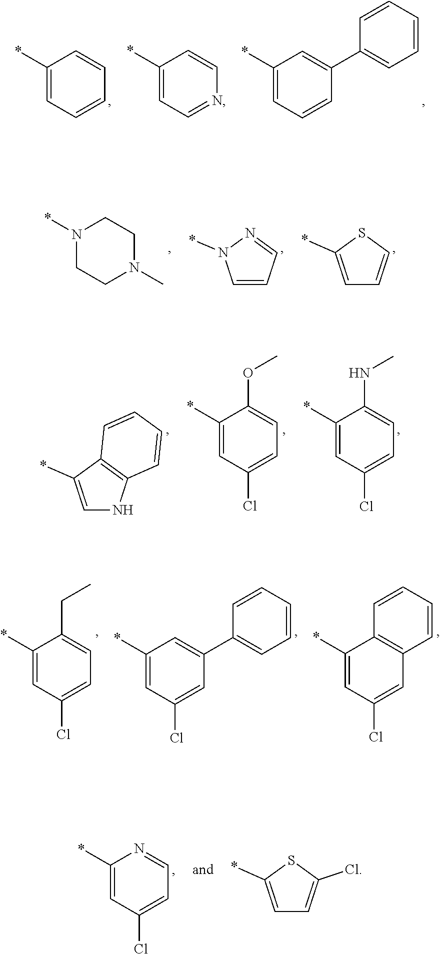

5. The compound of claim 1, wherein Y has a formula selected from:

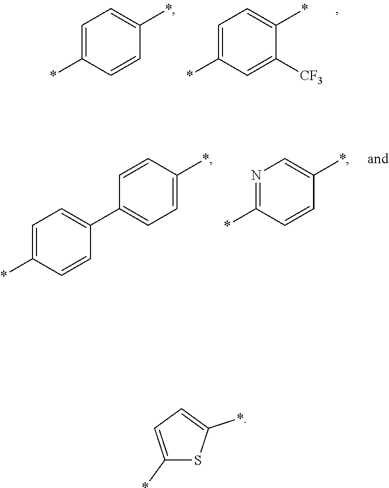

6. The compound of claim 1, wherein Z has a formula selected from:

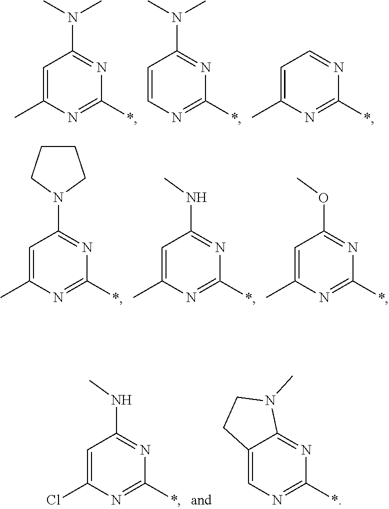

7. The compound of claim 1, wherein R¹ is hydrogen.

8. The compound of claim 1, wherein R¹ is methyl.

9. The compound of claim 1, wherein R² is hydrogen.

10. The compound of claim 1, wherein R² is methyl.

11. The compound of claim 1, wherein R³ and R^{3′} together form oxo.

12. The compound of claim 3, wherein R⁴ has a formula, 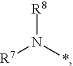
wherein R⁷ and R⁸ are hydrogen, methyl, or a moiety comprising a biotinyl group.

13. The compound of claim 1, wherein X is a phenyl group optionally substituted at one or more ring positions with one or more alkyl, alkoxy and halo.

14. The compound of claim 1 wherein: m is 0 or 1; n is 0 or 1; and/or p is 0 or 1.

15. The compound of claim 3, wherein R¹ is methyl, R⁴ has a formula 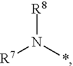
wherein R⁷ and R are methyl; and X is phenyl, optionally substituted with one or more alkyl, alkoxy and halo.

16. The compound of claim 1 having a formula selected from:

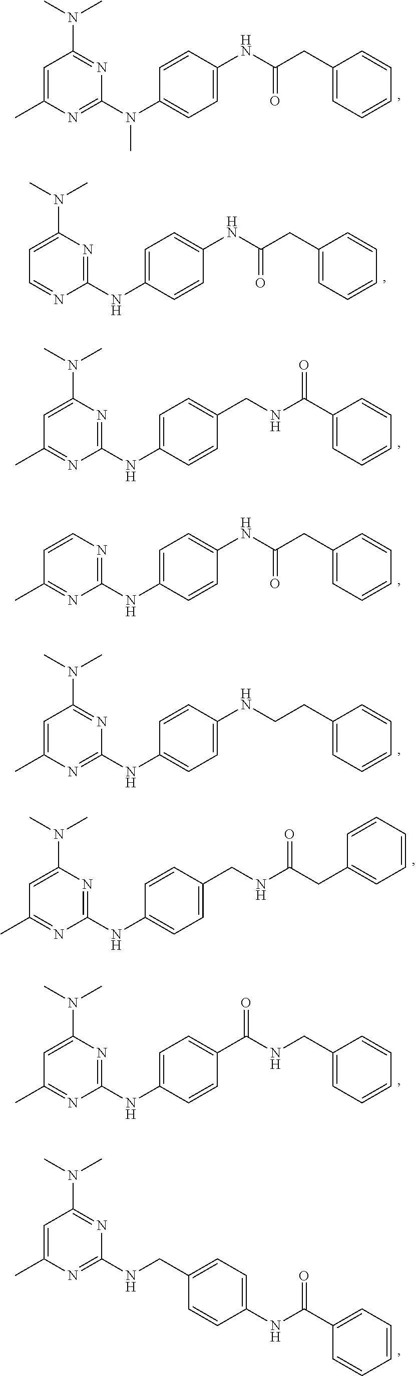

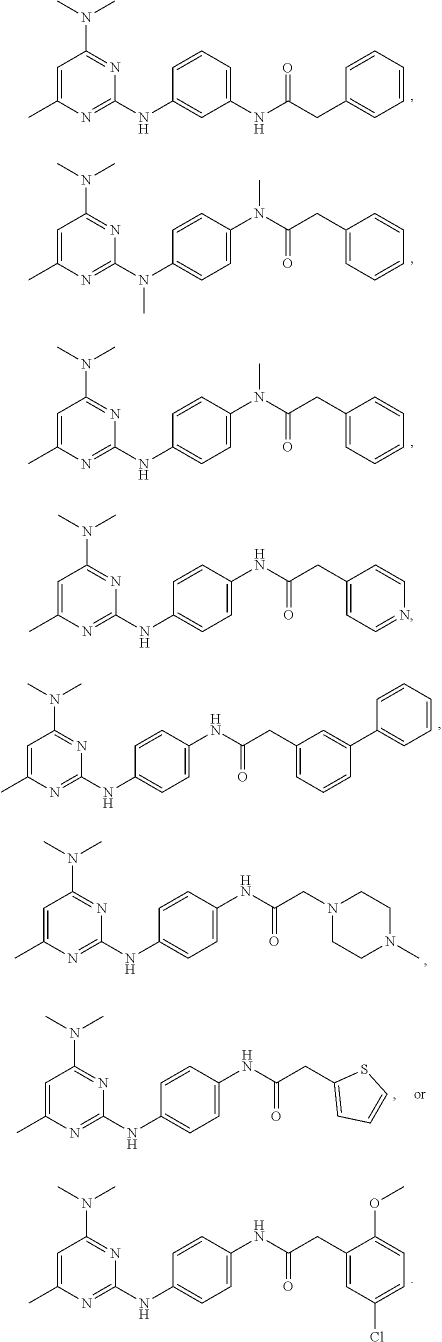

17. A pharmaceutical composition comprising the compound of claim 1.

18. The pharmaceutical composition of claim 17, wherein the compound has a formula selected from:

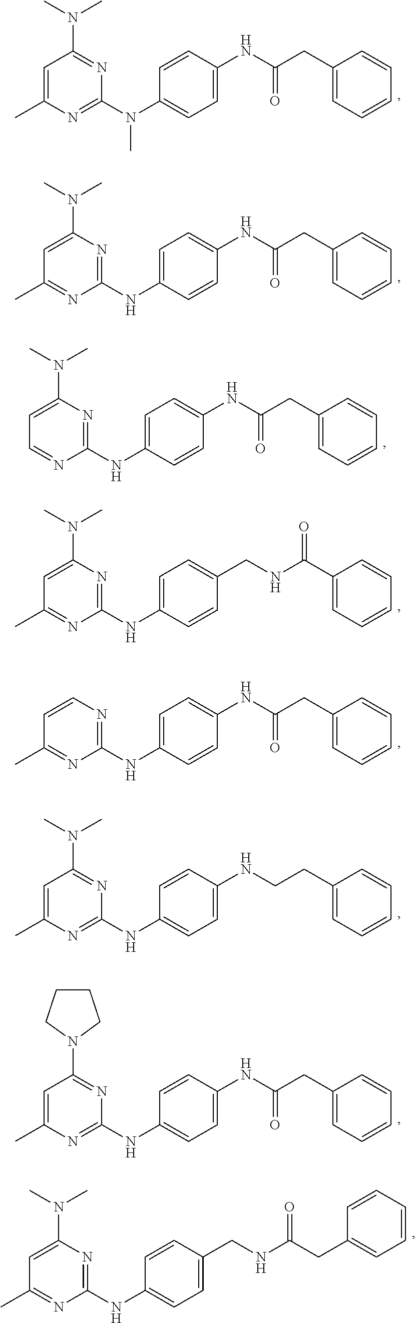

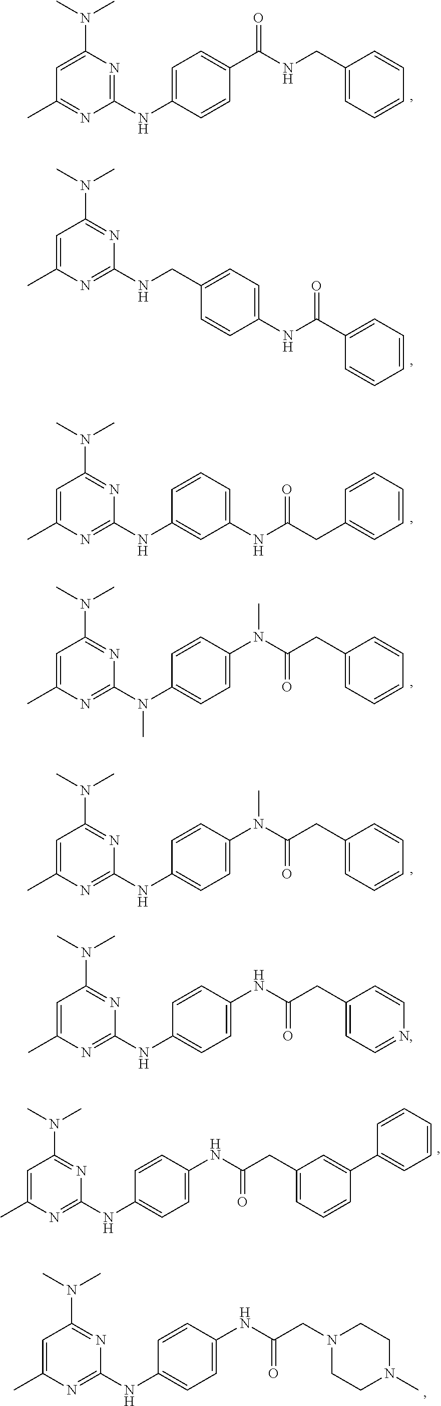

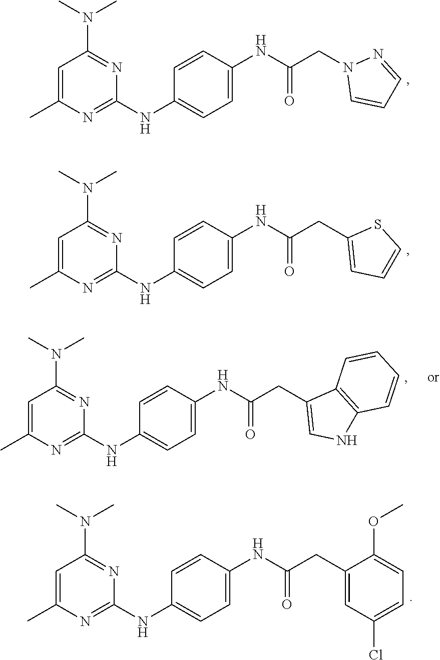

19. A method of treating or preventing a disease or disorder associated with tissue transglutaminase in a subject in need thereof, the method comprising administering to the subject of the compound of claim 1.

20. The method of claim 19, wherein the disease or disorder is a cell proliferation diseases or disorder.

21. The method of claim 20, wherein the cell proliferation disease or disorder is cancer.

22. The method of claim 20, wherein the cell proliferation disease or disorder is ovarian cancer.
